(19) United States
(12) Patent Application Publication
(10) Pub. No.: US 2004/0078231 A1
(43) Pub. Date: Apr. 22, 2004

(54) System and method for facilitating and administering treatment to a patient, including clinical decision making, order workflow and integration of clinical documentation

(76) Inventors: Gordon J. Wilkes, Newmarket (CA); Eric S. Paul, North York (CA); Meetali Acharya, Burlington (CA); Sayeh Radpay, Toronto (CA)

Correspondence Address: Francis J. Kowalik, Esq., Corporate Counsel, Law Department, BAXTER INTERNATIONAL INC., One Baxter Parkway, DF3-2E, Deerfield, IL, 60015, US

(21) Appl. No.: 10/452,822

(22) Filed: Jun. 2, 2003

Related U.S. Application Data
(60) Provisional application No. 60/385,176, filed on May 31, 2002.
(60) Provisional application No. 60/384,717, filed on May 31, 2002.
(60) Provisional application No. 60/384,607, filed on May 31, 2002.
(60) Provisional application No. 60/401,899, filed on Aug. 7, 2002.

(51) Int. Cl. G06F 17/60; A61B 5/00

(52) U.S. Cl. 705/2; 705/3; 600/300

(57) ABSTRACT

A system and method is disclosed for utilizing electronic clinical documentation, including nursing orders, patient treatment orders, and systems and methods for providing decision support, including clinical decision support, during the treatment of a patient.

### RELATED APPLICATIONS

The present application claims the benefit of the following U.S. Provisional Applications: “Nursing Orders Workflow System And Method,” Serial No. 60/385,176, filed May 31, 2002; “System And Method For Supporting Clinical Decisions During Patient Care Treatment,” Serial No. 60/384,717, filed May 31, 2002; and, “System And Method For Facilitating Orders During Patient Care Treatment,” Serial No. 60/384,607, filed May 31, 2002. Each of these provisional applications are herein incorporated by reference.

The present application also incorporates by reference pending U.S. application “System And Method For Integrating Clinical Documentation With The Point of Care Treatment of a Patient,” Ser. No. 10/427,374, filed Apr. 30, 2003.

### TECHNICAL FIELD

This invention relates generally to patient care. More specifically, the present invention is directed to a clinical documentation system and method, such as a system and method for integrating clinical documentation including nursing orders, patient treatment orders, and systems and methods for providing decision support, including clinical decision support, during the treatment of a patient.

### BACKGROUND OF THE INVENTION

Healthcare facilities aim to provide high quality patient care. To ensure that high quality patient care is provide, each healthcare facility utilizes defined policies and procedures. Furthermore, each healthcare facility utilizes patient care systems.

One such aspect of a patient care system concerns patient charting. Patient charting is made difficult because it is common today for an individual receiving patient care from a health care facility to require multiple visits to the facility, and potentially to different facilities. While some of the visits may be unrelated and pertain to isolated incidents, other visits may involve a specific patient treatment. Continuous patient care may span several visits and the medication and/or treatment prescribed to the patient are often the same on each visit. Moreover, many health care facilities today utilize paper-based patient charts. The paper charts require health care personnel to write by hand all orders or documentation related to an encounter between the patient and the health care facility. Although a current encounter may contain many similar characteristics of a previous encounter, the paper-based charts do not easily allow for copying orders or documentation from previous to current encounters. It is necessary for health care personnel to completely re-write the order or documentation related to the current encounter. Inaccuracy and human error may occur during the manual transcription of past data on a patient's chart to the current encounter. Erroneous documentation within the health care facility can lead to unsafe and costly patient care treatment.

Patient charting, however, is extremely useful in monitoring treatment to provide quality patient care. The structure of the patient chart typically conforms with the facility's policies and procedures.

The facility's guidelines, however, are continually evolving and established practices are in place for appropriately proceeding with any changes. Once approved, notification of the changes is dispersed to personnel throughout the healthcare facility. Typically, healthcare personnel are notified through meetings and memos shortly after the changes have been allowed. The notification may be delayed or fail to reach everyone due to work absences, schedule conflicts, etc. It is possible that a significant amount of time can pass until the facility is completely operating under the changed policy and/or procedure. Additionally, some policy changes may affect the paper-based patient chart. Again, a significant period of time may pass before any changes are incorporated into the patient chart. Furthermore, any diversion of the established procedures, whether justified or not, are not easily monitored or tracked because many tracking attempts are ineffective.

Policy changes requiring extended transition periods can result in multiple and/or inaccurate treatment processes being followed within the healthcare facility. Such disorder can lead to unsafe and costly patient care treatment.

In addition to patient charts, most patient care systems typically include computer networks, medical devices for treating a patient, and controls for the medical devices. Although patient care systems have been improved through the use of computerized automation systems and methods, patient care systems continue to rely heavily upon manual data management processes for medical devices and controls for medical devices. For example, nursing stations are typically connected to the computer networks in modern hospitals, but it is unusual for the computer network to extend to a patient's room. Computer networks offer the opportunity for automated data management processing including the operating and monitoring of medical devices and controls for the medical devices at the point-of-care. Despite advances in the field, automated data management technology has been underutilized for point-of-care applications due to a lack of more efficient systems and methods.

Errors can be attributed to a number of things between when a clinician recognizes the need for a treatment and when the treatment is administered to a patient. As explained above, paper charts and medical administrative records (MARs) have traditionally been used to coordinate the treatment decision process and the resulting treatment. However, creating and using paper MARs is a process that is prone to errors. Paper MARs are generally not verified against system-wide treatment standards. Every clinician may create a MAR in a slightly different manner. Variability in the creation of MARs leads to errors in interpretation of the MARs. Different clinicians may not be aware of what other clinicians are doing in regard to the treatment of the patient. Ultimately, paper MARs result in errors in the treatment administered to patients. One place where these errors are particularly dangerous is in the administration of medical treatment involving medications. It would be beneficial to have an improved system for creating and using MARs to administer medical treatment.

The present invention is provided to solve these and other problems.

### SUMMARY OF THE INVENTION

One embodiment of the present invention is directed to a system and method for facilitating accurate ordering and documentation within a health care facility. The system is configurable by a health care facility and utilizes prior orders and/or documentation for quick and accurate creation of new orders. The health care facility is capable of configuring the system as desired wherein access to the system is monitored and controlled. The system includes a processor and a memory. A record of encounters is maintained in the memory and accessible to the processor. A form for configuring the system is provided to the health care facility to be populated. The processor utilizes the populated form to generate a copy-forward function. The copy-forward function is provided to authorize users for quickly and accurately utilizing existing orders as an origin, e.g., template, for a new order.

Through the present invention a health care facility may be able to more accurately and effectively enter orders or documentation. Additionally, the efficiency and productivity of patient care within a health care facility may be enhanced.

Another embodiment of the present invention is directed to a system and method for providing decision support to healthcare personnel during treatment of a patient. The system comprises an event having at least one patient characteristic. A search module cooperates with a processor and a memory to uncover a patient record stored in the memory and matching the event. An alert is activated in response to the search module wherein guidance is provided to healthcare personnel during treatment.

Yet another embodiment of the present invention concerns the efficient and effective workflow of nurses within a hospital for providing care to the patients. In this embodiment, hospitals are able to order nursing activities and procedures with schedule creation. One embodiment of the schedule creation invention would schedule the nursing task regardless of any other patient care related events. Additionally, another embodiment of the schedule creation task would not schedule the event until another event or series of events have been completed. The nursing order is mapped to one or more structured data elements for the purposes of clinical documentation. The system at the appropriate times according to the schedule generated for the nursing task(s), alerts the nurse of the clinical documentation data element(s), which have been scheduled through nursing orders and require data capture. Integration of scanning technology into the process is optional. This includes scanning a patient's bar-coded wristband thereby ensuring the correct patient and scanning a bar code identifying the task/procedure to automatically chart completion.

One embodiment of the nursing workflow and event management would occur in real-time in a wireless environment. Furthermore, it may be designed to function on a workstation, a tablet, and/or a PDA.

Prior practice concerning orders is disjointed in nature and does not ensure closing the loop on nursing care. Many of these shortcomings are due to the deficiencies of a paper-based environment. Physicians prescribe orders, and intertwined in those orders are nursing activities and procedures. Often these are not explicitly stated/documented, but instead remain in the clinician's head to remember appropriate procedures to be performed. In some advanced institutions, patient care plans may be documented on an electronic system; however, the insurance that the care plan is followed through scheduled workflow management on a wireless handheld device does not exist. Furthermore, documentation of results of nursing patient-related activities, procedures, and plans is a source of frustration. The time to complete documentation is lengthy and the amount of information required in today's regulated environment is cumbersome. Sometimes, due to lack of time and appropriate resources for documentation, documentation goes amiss.

In the present invention, various features may be incorporated, including: (a) documentation of nursing tasks, activities and procedures, including care plans on the patient's electronic chart, may be integrated with the patient's order profile; (b) automatic schedule generation to include nursing-related tasks according to their patient's order profile; (c) electronic “to do” lists may be provided in real-time to assist with completing tasks in a timely fashion; (d) automatic alerting of patient documentation requirements where appropriate (this provides nurses with quick patient bedside documentation tools to help maintain compliance with regulatory issues); and, (e) integration of the nursing patient care process (this may be accomplished by integrating medication management by using bar coding and scanning technology during order entry, dispensing, and administration with other nursing activities and procedures).

Another feature of the present invention includes mapping. This may include mapping of items, tests and procedures to structured clinical documentation data elements, groups, and/or sets. Additionally, a preferred embodiment of the present invention provides for “workflow mapping.” The following may be considered variants or equivalents of the invention: (a) providing an integrated nursing workflow process, but not in a wireless environment; (b) combining disparate systems to achieve the total integrated nursing workflow effect (i.e., combining an order entry system with a care plan system with a documentation system); (c) extending the orders workflow concepts to other disciplines such as respiratory therapists, lab technicians, physiotherapists, etc. (d) sending all patient related data and schedule to a clinical data repository, which proceeds to manage the workflow in the same concepts described above; and, (e) providing parts of the workflow management process. but not all. An example may be a system integrating task scheduling with an electronic To Do List.

In yet another embodiment, the present invention provides a system and method for integrating clinical documentation with the point of care treatment of a patient within a healthcare facility, typically in connection with a patient care system.

Generally, this embodiment of the present invention provides a clinical documentation application or module including an end-user forms utility that enables a healthcare organization to customize patient documentation within a patient care system. The utility allows the healthcare organization to define “n” data elements—according to pre-defined data types—that can be utilized in groups and sets. Additional features can be added to a hospital-definable meta-data system to allow for user entry validation, formatting, end user decision support and rules. The rule-driven clinical documentation provides clinicians real-time decision support at the point of care. The present invention provides several hospital-definable formatting options for clinical documentation forms available at a workstation, tablet, PDA, or other device associated with the patient care system. The system and method of the present invention can also be implemented, in whole or in part, utilizing wireless technology.

In such a system, real-time, electronic access to documentation results is available anywhere and anytime. As soon as documentation is entered (e.g., through a handheld device) physicians and other healthcare personnel (e.g., clinicians, pharmacists, nurses) have access to the information for making patient care related decisions accordingly and in a timely manner. In the past, patient result documentation occurred on many disparate systems. For example, lab results came from a laboratory system, some patient documentation was put on a paper chart while other information was found in electronic systems. Coordination of the information requires the time and ability of a clinician to accumulate and sort through several types of stored information before analyzing the data required for patient care. By providing the ability to manage, coordinate and integrate all patient clinical documentation in a hospital-defined manner and in a single location—which is also readily accessible from a variety of locations—patient care is more efficient and accurate.

Other systems, methods, features, and advantages of the present invention will be, or will become, apparent to one having ordinary skill in the art upon examination of the following drawings and detailed description. It is intended that all such additional systems, methods, features, and advantages included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals typically, but not necessarily, designate corresponding parts throughout the several views.

FIG. 1 is a block diagram of one embodiment of the present invention;

FIG. 2 is a screen view depicting an aspect of the present invention of FIG. 1;

FIG. 3 is a screen view depicting an aspect of the present invention of FIG. 1;

FIG. 4 is a screen view depicting an aspect of the present invention of FIG. 1;

FIG. 5 is a block diagram of another embodiment of the present invention;

FIGS. 6a-6c are screen views of pages displayed during the creation of an event wherein demographic information (e.g., allergy and age) is added and defined with the event of the embodiment of FIG. 5;

FIGS. 6d-6g are screen views of pages displayed during the creation of an event wherein the type of alert is defined during the creation of an event of the embodiment of FIG. 5;

FIG. 7 is a graphical representation of a patient care system of the present invention. The patient care system includes a pharmacy computer, a central system, and a digital assistant at a treatment location;

FIG. 8 is a block diagram of a computer system that may be representative of the pharmacy computer, the central system, and/or the digital assistant of FIG. 7;

FIG. 9 is a block diagram showing functional components of the patient care system of FIG. 7;

FIG. 10 is an exemplar computer screen that is useful in implementing various functions of the patient care system of FIG. 7;

FIG. 11 is a block diagram showing functional components of the infusion system of FIG. 8;

FIG. 12 is a block diagram showing functional components for the setting of infusion system parameters of FIG. 11;

FIG. 13 is a block diagram showing functional components for the infusion order creation of FIG. 11;

FIG. 14 is a block diagram showing functional components for the infusion order preparation of FIG. 11;

FIG. 15 is a block diagram showing functional components for the medication administration of FIG. 11;

FIG. 16 is a block diagram showing functional components for infusion order documentation, and the infusion order modifications and messaging of FIG. 11;

FIG. 17 is a block diagram of an embodiment of a system for integrating structural clinical documentation with point of care treatment of a patient within a healthcare facility;

FIG. 18 is a diagram of an exemplar screen utilized in connection with the present invention;

FIG. 19 is a diagram of an exemplar patient information panel portion of the screen of FIG. 18;

FIG. 20 is a diagram of an exemplar navigation bar portion of the screen of FIG. 18;

FIG. 21 is diagram of an exemplar menu panel portion of a screen displaying available user options in accordance with a particular aspect of the present invention;

FIG. 22 is a diagram of an exemplar physician information summary screen in accordance with a particular aspect of the present invention;

FIG. 23 is a diagram of an exemplar message screen in accordance with a particular aspect of the present invention;

FIG. 24 is a diagram of an exemplar read message screen in accordance with a particular aspect of the present invention;

FIG. 25 is a diagram of an exemplar send message screen in accordance with a particular aspect of the present invention;

FIG. 26 is a diagram of an exemplar screen depicting patient demographic information in accordance with a particular aspect of the present invention;

FIG. 27 is a diagram of an exemplar patient information summary screen in accordance with a particular aspect of the present invention;

FIG. 28 is a diagram of an exemplar patient clinical documentation screen in accordance with a particular aspect of the present invention;

FIG. 29 is a diagram of an exemplar sort screen for the patient clinical documentation screen of FIG. 28;

FIG. 30 is a diagram of an exemplar screen depicting additional information of the patient clinical documentation screen of FIG. 28;

FIG. 31 is a diagram of an exemplar screen for creating new documentation in accordance with a particular aspect of the present invention;

FIG. 32 is a diagram of an exemplar disease state profile screen in accordance with a particular aspect of the present invention;

FIG. 33 is a diagram of an exemplar disease state look-up screen in accordance with a particular aspect of the present invention;

FIG. 34 is a diagram of an exemplar disease state history screen in accordance with a particular aspect of the present invention;

FIG. 35 is a diagram of an exemplar allergy profile screen in accordance with a particular aspect of the present invention;

FIG. 36 is a diagram of an exemplar search window for recording a new item allergy in accordance with a particular aspect of the present invention;

FIG. 37 is a diagram of an exemplar window displaying a listing of allergy profile in accordance with a particular aspect of the present invention;

FIG. 38 is a diagram of an exemplar window for selecting an allergy in accordance with a particular aspect of the present invention;

FIG. 39 is a diagram of an exemplar allergy history screen in accordance with a particular aspect of the present invention;

FIG. 40 is a diagram of an exemplar order profile screen in accordance with a particular aspect of the present invention;

FIG. 41 is a diagram of an exemplar window for creating a sort order for the order profile screen of FIG. 40;

FIG. 42 is a diagram of an exemplar order detail pop-up window in accordance with a particular aspect of the present invention;

FIG. 43 is a diagram of an exemplar window listing order profiles requiring authorization in accordance with a particular aspect of the present invention;

FIG. 44 is a diagram of an exemplar window for providing authorization of the order profile in accordance with a particular aspect of the present invention;

FIG. 45 is a diagram of an exemplar order entry window in accordance with a particular aspect of the present invention;

FIG. 46 is a diagram of an exemplar window for searching an order entry in accordance with a particular aspect of the present invention;

FIG. 47 is a diagram of an exemplar window for adding orders to a patient's order profile in accordance with a particular aspect of the present invention;

FIG. 48 is a diagram of an exemplar window listing changes made to an order profile in accordance with a particular aspect of the present invention;

FIG. 49 is a diagram of an exemplar window for the Rx Dose Utility in accordance with a particular aspect of the present invention;

FIG. 50 is a diagram of an exemplar editing window for an order profile in accordance with a particular aspect of the present invention;

FIG. 51 is a diagram of an exemplar medication alert window in accordance with a particular aspect of the present invention;

FIG. 52 is a diagram of an exemplar drug therapy alterations window in accordance with a particular aspect of the present invention;

FIG. 53 is a diagram of an exemplar results window in accordance with a particular aspect of the present invention;

FIG. 54 is a diagram of an exemplar monitoring window in accordance with a particular aspect of the present invention;

FIG. 55 is a diagram of an exemplar test results window in accordance with a particular aspect of the present invention;

FIG. 56 is a diagram of an exemplar window showing details of the test results window of FIG. 55;

FIG. 57 is a diagram of an exemplar medications results window in accordance with a particular aspect of the present invention;

FIG. 58 is a diagram of an exemplar order template window in accordance with a particular aspect of the present invention;

FIG. 59 is a diagram of an exemplar window for creating an order template in accordance with a particular aspect of the present invention;

FIG. 60 is a diagram of an exemplar order timing utility window in accordance with a particular aspect of the present invention;

FIG. 61 is a diagram of an exemplar order set templates window in accordance with a particular aspect of the present invention;

FIG. 62 is a diagram of an exemplar window for creating an order set template in accordance with a particular aspect of the present invention;

FIG. 63 is a diagram of an exemplar patient clinical documentation window in accordance with a particular aspect of the present invention;

FIG. 64 is a diagram of an exemplar computer physician order entry window in accordance with a particular aspect of the present invention; and,

FIG. 65 is a diagram of an exemplar second computer physician order entry window in accordance with a particular aspect of the present invention.

### DETAILED DESCRIPTION

While this invention is susceptible of embodiments in many different forms, there is shown in the drawings and will herein be described in detail a preferred embodiment of the invention. The present disclosure is to be considered as an exemplification of the principles of the invention and is not intended to limit the broad aspect of the invention to the embodiment illustrated.

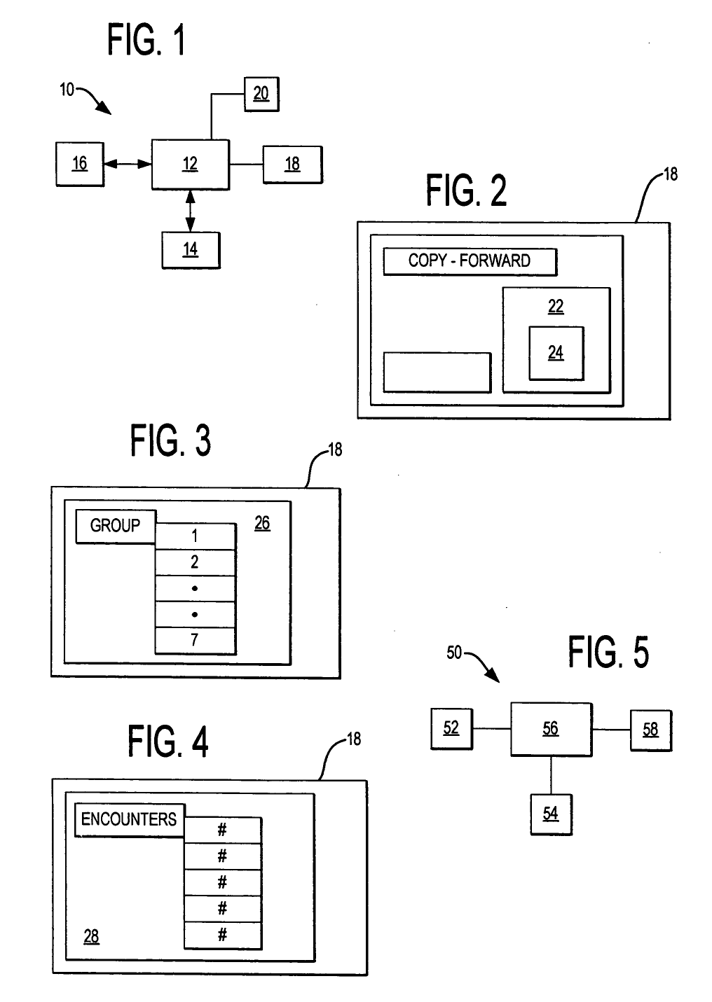

One embodiment of the present invention, as shown in FIGS. 1-4, is associated with a point of care system that includes a longitudinal patient record spanning a patient's continuum of care. The patient record includes, and is not limited to various encounters, e.g., out-patient clinic visits, emergency visits, hospital in-patient visits, etc. When patients are diagnosed with chronic illnesses, many times the procedures and/or treatments ordered and/or documented during an encounter episode are similar in nature. To increase the continuity of care throughout the health care facility, the present invention provides for viewing and selecting existing encounter orders and patient documentation for copying and forwarding into a current encounter or for creating a base order, e.g., a working template, for a new encounter order. The ability to use existing orders as a starting point for generating a new order will reduce the amount of time to create a new order. Furthermore, potential errors typically resulting from manual entry may be reduced or eliminated. Additionally, the clinician has the ability to edit the order, activate the order, and/or authorize the order according to hospital-definable rules.

One means for creating new encounter orders based no working templates is with a “copy-forward” function. Access and applicability of the “copy-forward” functionality is conditional upon guidelines set within the health care facility. The health care facility configures the “copy-forward” functionality in accordance with these rules. User and order accessibility is defined by the health care facility. Ultimately, health care personnel viewing a patient's electronic chart will be allowed to select existing encounter orders to be copied into a current encounter. After an order has been selected, health care personnel are able to edit the order, activate the order, and execute the order. The permission status of the user and order are determined by the health care facility during configuration of the “copy-forward” functionality. Some benefits of the present invention may include: increased patient safety, increased continuity of care, and enhanced efficiency and productivity. In addition to allowing existing orders to be accessed and utilized, a standardized order, or order set, can be provided to health care personnel for creating a new order.

Many times, patients with a long or complicated medical history have extensive record charts. The large quantity of information is extremely difficult to review. Additionally, the large quantity of information makes it difficult to search clinical patient data, e.g., a patient's particular diagnosis/illness. One aspect of the present invention involves providing a clinician the ability to view existing, or past, encounters involving medication orders of a patient's profile and selecting existing orders to be copied into a new, current encounter.

It is common for a health care facility implementing such a system to incorporate a variety of user groups, e.g., pharmacists, nurses, doctors, interns, residents, lab technicians, nursing assistants, etc. Furthermore, the health care facility generally includes many disciplines, such as pharmacy, nursing, medical, laboratory, radiology, dietary, physiotherapy, etc. Within each discipline, order types exist that are also definable by the health care facility. One order type associated with the pharmacy discipline may include: piggybacks, large volume parenterals, adjusting doses, alternating infusions, sequencing infusions, etc. The present invention enables the health care facility to define which user group, discipline, and order type may be permitted access to the copy-forward functionality. For example, the health care institution may permit utilization of the copy-forward function in relation to medication orders, but not radiological orders.

The health care facility is provided the ability to define which personnel will have access to the copy-forward functionality. Similarly, the facility is capable of excluding certain encounters or orders from being utilized with the copy-forward function. Furthermore, time-based limits can be assigned to the copy-forward functionality wherein of an existing encounter must be copied within a predetermined time period. The time period is defined during configuration of the copy-forward function.

It is to be understood that some patient information may be automatically copied and forwarded into a current encounter each time a patient is admitted to the health care facility. The facility will determine which data elements of a patient's electronic chart are to be automatically copied forward with each encounter. If automatic copying is enabled, the facility may also decide whether a clinician should perform a physical verification of the automatically copied data. If the facility indicates that physical verification should be performed, facility personnel may be notified through an information summary. The automatically copied data is identified as such in some manner, e.g., visual, until verified by a clinician. Once verified, the manner of identification will disappear and a record, e.g., audit trail, of the verification will be kept in memory.

By way of example and not limitation, FIG. 1 depicts one embodiment of the present invention wherein a system 10 includes a configuration module 16 for configuring the copy-forward application. The configuration module 16 is operably connected to a processor 12, a memory 14 and an interface 20. The configuration module 16 includes a form 22 to be provided during configuration of the copy-forward function. The health care facility utilizes the configuration module 16 to define user, discipline and order accessibility to the copy-forward function. As shown in FIG. 2, during configuration of the copy-forward function, the copy-forward form window 22 appears on a display 18. The form 22 contains a table 24 with “+” and “−” signs next to a description to allow copy-forward of medication orders. Selection of the “+” sign will open a Groups table window 26, shown in FIG. 3, and allow single or multiple selection of the Groups in the facility. Users within the selected Group will be allowed to copy medication orders to other encounters for a given patient. If a Group is not present in the table, members of the Group will not be allowed access to the copy-forward function. The contents, e.g., members, of the Group, as well as the inclusion of the Group itself, is determinable by the facility during configuration of the copy-forward function.

Additional aspects of the copy-forward functionality can also be defined. In particular, the copy-forward function can be limited with respect to time wherein an encounter is not allowed to be copied if it has existed for a predetermined amount of time. To implement such a restriction, a line edit field to the copy-forward form window is included wherein a drop-down menu comprising a list of time units, e.g., minutes, hours, days, weeks, and months, will appear upon selection of the menu item. The time specified in this field indicates the length of time not to be exceeded by existing medication orders for copying.

Yet another definable aspect of the copy-forward functionality involves order types, more specifically, the exclusion of an order type. During configuration of the copy-forward functionality, a drop-down field appears upon selection of the copy-forward item. The drop-down field will list all order types specified in an order types reference code table. Preferably, the drop-down field allows for multi-selection of order types. The system will not allow copy-forward of orders that are of an order-type selected in the drop down field. If copy-forward of one of these order types is attempted, a message will be displayed stating that copy-forward is prohibited.

Once configured, the copy-forward function is incorporated into various electronic health care systems. The copy-forward function can also be embodied into the electronic system as a pull-down or drop-down item in a menu. Preferably, any attempt by health care personnel to utilize the copy-forward function is validated by the system to ensure that the personnel is authorized access to copy orders. If the user or group is not authorized for such activity, a message will be displayed stating that access to the copy-forward function is denied. Any order type selected by a user for use with the copy-forward function can be validated by the system. Order types that are excluded from the copy-forward feature will be identified and prevented from being utilized with the copy-forward function. Users will not be able to copy order types that are excluded from the copy-forward feature. If an excluded order type is selected, a message stating that the order cannot be copied to another account will be displayed.

An end user's interaction with the configure copy-forward function begins with selection of the function. As shown in FIG. 4, once the copy-forward function is selected, a patient encounter lookup window will appear 28 and prompt the user to select an encounter order to copy. In addition, any orders that exceed the time limit defined during configuration will not be displayed as available for copying. Once an encounter is selected and copied, the processor 12, e.g., infusion wizard or the Rx dose wizard, is triggered with respect to the selected order type. In the infusion or Rx dose wizard, each order will be displayed as if it was selected to be modified in the current encounter.

Preferably, selected order(s) are copied in sequence. Similar to “newly created orders,” each order created with the copy-forward function is required to be validated by its respective wizard prior to being saved in memory. Similar to placing a new order/encounter, validation and/or screening is performed prior to placing the order. All successfully copied orders are displayed in a patient's Rx profile and maintained in the system's memory.

An alternative embodiment of the present invention incorporates a copy-forward hyperlink to a menu bar residing within an order profile screen. The hyperlink is provided to a user belonging to a group that has been granted access during configuration of the copy-forward function. Authorized users are able to copy one or multiple medication/infusion orders—preferably using a checkbox next to each order and selecting the copy hyperlink. This action will open a patient encounter lookup window and prompt the user to select the encounter for copying. The selected orders are validated to ensure that they do not exist in the selected encounter. Any order defined as accessible during configuration of the copy-forward function is capable of being selected for copying. The orders that are copied to a particular encounter can be displayed in a selected/current orders section of the order entry window.

During utilization of the present invention by a health care facility, the copy-forward function will be validated in accordance with the facility's rules prior to continuing execution. When provided, the user will have the ability to select medication orders and choose an existing encounter from which to copy. Preferably, the group of encounters provided for selection thereof have an active status. If only one active encounter exists, the sole active encounter is copied as a default. After copying the existing encounter into a new encounter, health care personnel may edit the encounter as necessary. Upon the user's selection to activate the new order, the order is validated in accordance with the predefined rules of the system.

A record of orders utilized in the copying between encounters is recorded and an audit trail is maintained in memory 14. The audit trail can include, but is not limited to: health care personnel involved with the creation of the new order; time and date of the copying; the encounter order from which the copying was performed; the encounter into which the copied order was directed; modifications made to the encounter order after being copied, etc.

It is further contemplated by the present invention that the copy-forward functionality be applicable to all areas of the patient electronic chart, including but not limited to: patient demographic information, structured clinical documentation, free-text clinical documentation, ancillary orders—nursing, physiotherapy, radiology, dietary, etc.

During completion of structured clinical documentation, a clinician has access to a predetermined number of results that were previously documented. According to a facility's rules involving data element, group, set, and patient service level, the clinician can be permitted access to the copy-forward functionality to copy existing results, e.g., previously recorded results, into the current clinical documentation. The clinician may edit the copied results before storing into memory.

As with encounter orders, utilization of the copy-forward functionality with respect to clinical documentation can be restricted at the discipline and group level. Furthermore, each data element, group, and set defined within structured clinical documentation will allow restriction of copy-forward functionality. Validation to ensure copy-forward functionality preferably occurs prior to the time a clinician enters structured clinical documentation. Likewise, the health care facility has the ability to indicate during definition of the categories/sub-categories for free-text clinical documentation whether clinicians are allowed to copy-forward data by category or sub-category. Additionally, health care facilities can define whether a patient demographic element captured in the system is permitted the copy-forward functionality.

The rules pertaining to execution of the copy-forward functionality may include setting up user-specific, user-group-specific, disease state specific, and/or unit level specific provisions. Similar to service level dependencies, health care administrators may define whether orders or clinical documentation can be copied forward depending on the unit, service, and/or disease state(s) of the patient.

The present invention is applicable to any part of a patient's electronic chart. This may include, but is not limited to: clinical documentation, progress notes, history and physical, order profile, and a patient's interdisciplinary results. And, the copy-forward functionality may be used to copy patient data from one patient's electronic chart to another patient's electronic chart. As further understood by one of ordinary skill in the art, application of the present invention can be implemented with medication orders (regular and infusions), ancillary orders, clinical documentation notes, monitoring parameters, medication administration, assessments, as well as other health care encounters.

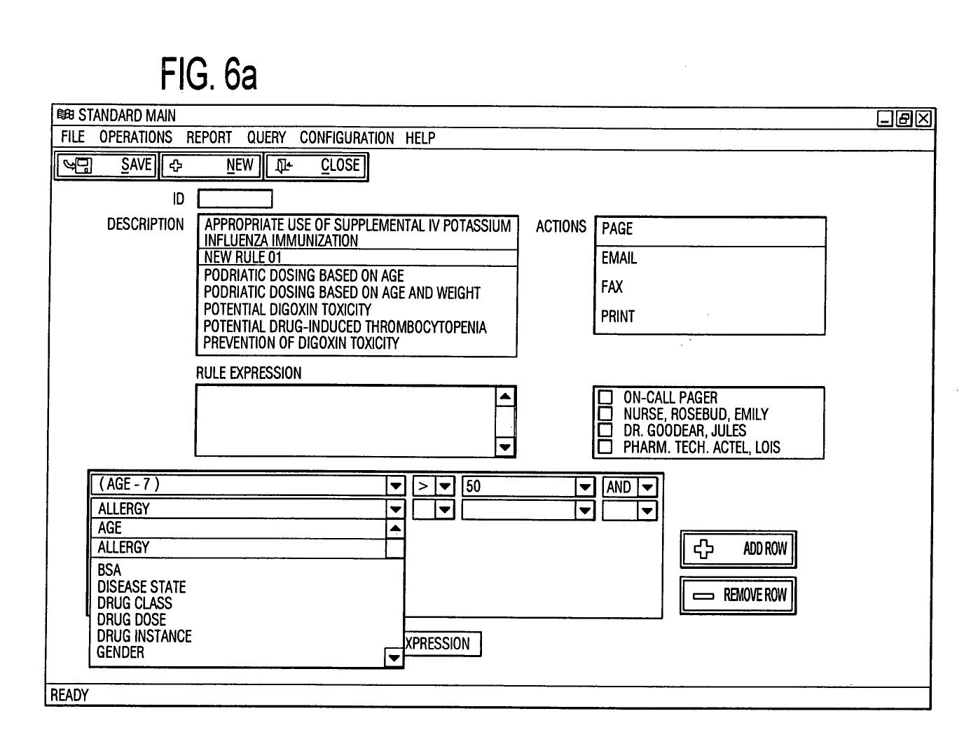

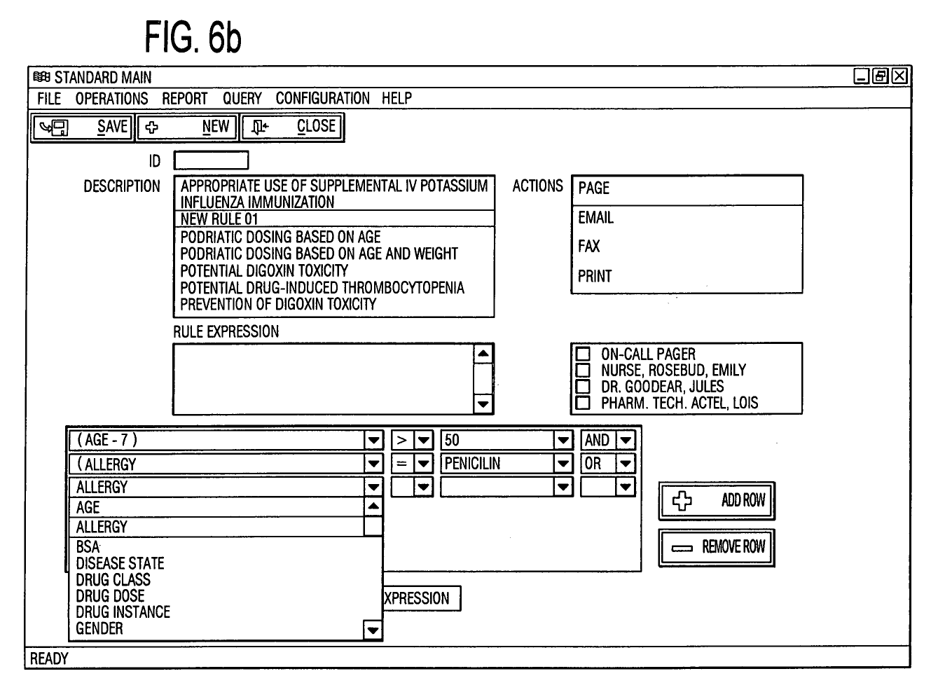

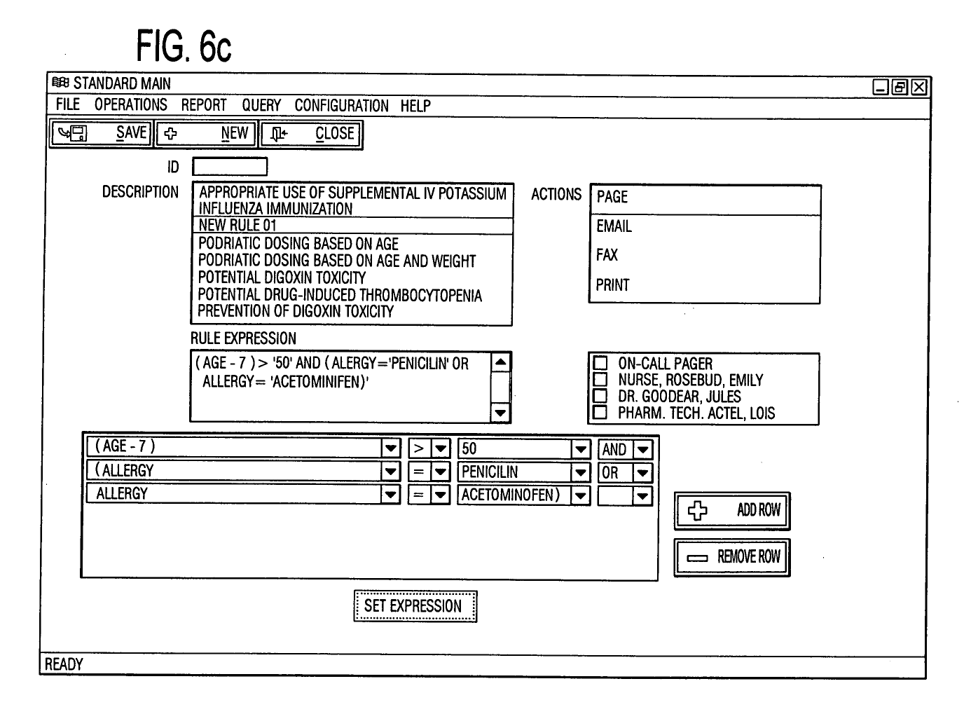

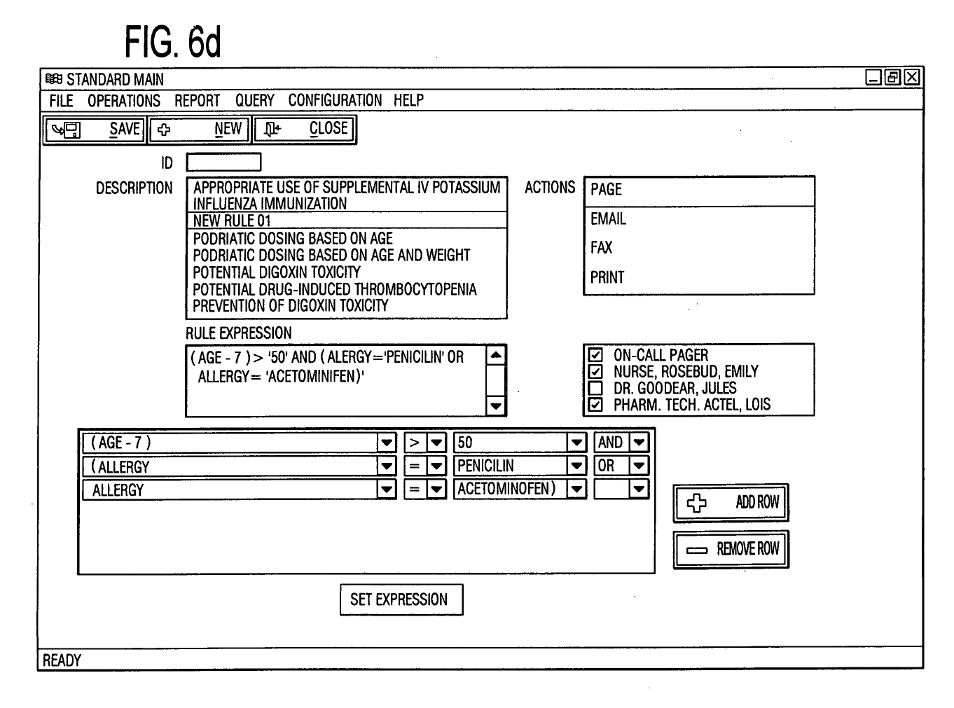

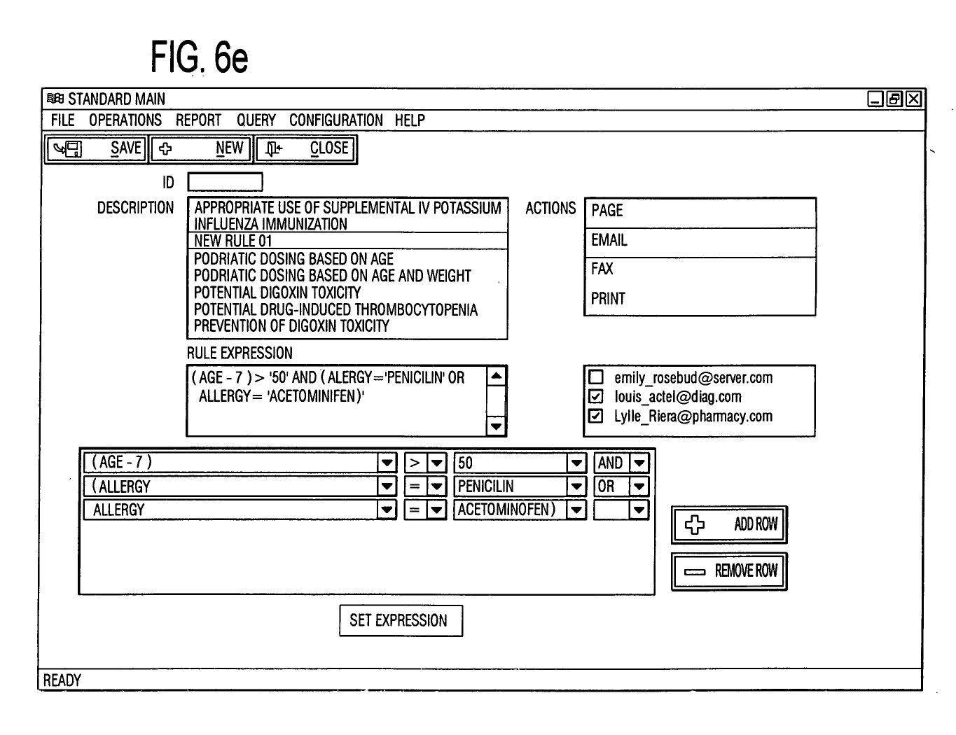

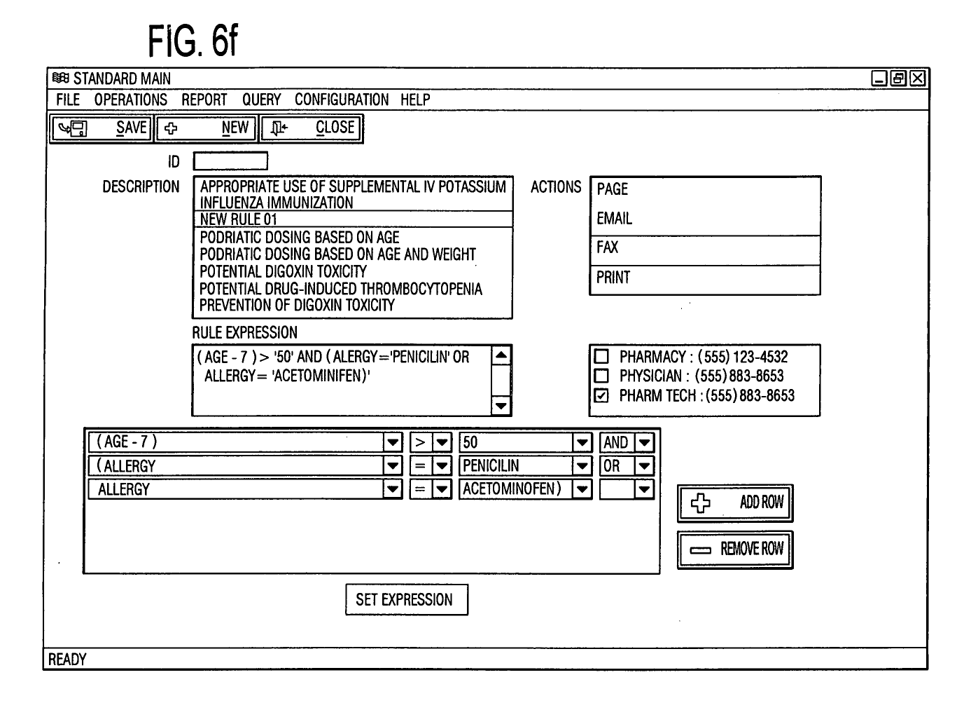

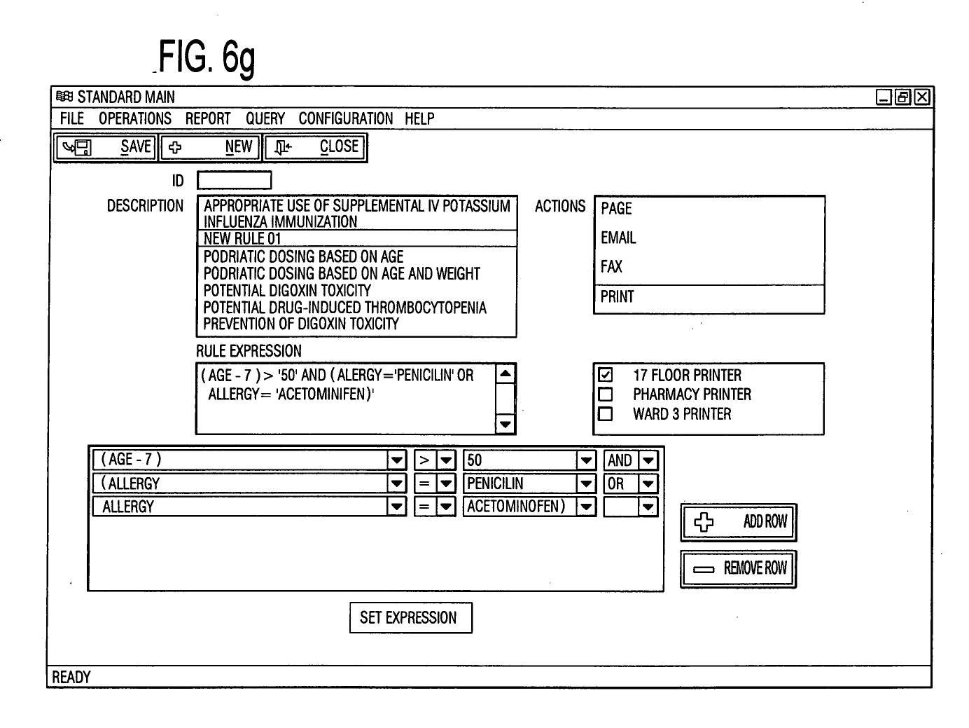

Another embodiment of the present invention, shown in FIGS. 5-6g, relates to a system and method for treating patients capable of cooperating with a rules engine to provide decision support at any point during treatment, e.g., patient chart review. Real-time support is provided to healthcare personnel through cross-disciplinary clinical-based alerts defined by a healthcare facility. The alerts are capable of being provided during order entries, medication or procedure administrations, nursing assessments, list evaluations of electronic patient charts, etc.

This embodiment can be readily integrated with patient workflow to provide quality patient treatment while maintaining healthcare standards and practices. The system 50 provides guidance during treatment of a patient. In one embodiment, various components of the system 50 may include an end-user programmable application or wizard 52, memory 54, a processor 56 and a user interface 58. The processor 56 cooperates with memory 54 to maintain patient and other clinical records. The records include various patient and other clinical characteristics. An event is defined and includes specified patient characteristics, clinical characteristics and records, laboratory records, medication records, disease states or records, etc. The wizard 52 includes a search module capable of searching through the memory for a record matching the event. An alert is activated in response to the identification of a matching record wherein decision support is provided to healthcare personnel to facilitate patient treatment.

The healthcare facility's policy is capable of being defined within the system to ensure performance of patient therapy within these principles. One means for accomplishing this is through use of the wizard 52 to define cross-disciplinary clinical-based alerts to quickly provide decision support during treatment of a patient. Any required deviations from the defined procedures can be tracked and reviewed.

Guidelines for monitoring therapy may be created using a variety of patient characteristics, including, but not limited to: demographic information such as gender, age, race, allergies, past medical history, present illness, risk factors for particular disease states, etc.; laboratory results; diagnostic results; vitals and other nursing-oriented monitoring and assessments; and medication ordering and administrations. The combination of this user-defined data is termed an event. FIGS. 6a-6c depict screen-shots of pages displayed at a user interface 58 during the creation of an event wherein age and allergy are utilized.

Each event can trigger an alert, e.g., indicator or evaluator, and several options are available for customizing the clinical alert. For example, one type of indicator may simply retrieve the requested event at automated times or upon demand, and can, if desired, provide a window containing a text-based message. FIGS. 6d-6g depict screen-shots of pages displayed at the user interface 58 during the creation of an event wherein different types of alerts are defined, e.g., page, e-mail, fax, and print.

An alert may also include a therapeutic medical decision or evaluation to change or add to some part of a patient's regimen. This may include, depending upon the type of event triggered, recommendations to the patient's medication profile, such as supplementing potassium and magnesium to prevent cardiac arrhythmia due to Digoxin toxicity, re-ordering of laboratory tests, or monitoring blood pressure. For example, an event may be defined as: male; greater than 60 years old; disease state of congestive heart failure; administered Digoxin; potassium level less than 3.0 mEq/L; and magnesium level of less than 0.99 mEq/L. This event will continually search the healthcare facility's patient database 60 to detect any patients who fit all defined logic of the event and an alert is triggered upon discovering an event match.

Upon access to the system, each user, e.g., physician, is presented with a customized view. The view summarizes outstanding clinical issues for each patient and enables quick retrieval of detailed information. Additionally, the system can also be configured to automatically produce an e-mail or page to a personal digital assistant device when certain critical patient conditions exist.

One aspect of the present invention involves age based drug-dosing and drug-lab alerts. The system provides the healthcare facility with an ability to define age categories. For each age category, the healthcare facility can define the appropriate dosing of each drug within the facility.

The system further includes an ability to define at a drug or lab test level which respective labs or drugs may be flagged for an alert based on age and or dose during order entry. The lab or drug results are received from administration and/or an ancillary laboratory system.

A further aspect of the present invention includes a wizard 52, definable by the healthcare facility, that allows definition of rules and alert messaging. The wizard 52 enables the healthcare facility to select one or multiple patient data elements, preferably stored within the system's memory 54, or another interfaced facility, to define parameters for these data elements at which an alert will be triggered. The alert message and its characteristics—to whom, when, where etc—are defined by the facility. In one embodiment of the present invention, the rules engine will not automatically perform clinical actions based on the combination of parameter set data elements. Instead, the rules engine sends a message advising the clinician. Additionally, the facility defined alert message may include clinical guidelines or suggestions, but will not be automatically executed. Alternatively, a change to patient therapy, as a result of an alert, may be completed automatically according to clinician authority, or, pending authorization by a clinician with authority and/or the determination of the patient's current status.

An ability to define patient age categories is provided by the system of the present invention. The following data elements can be utilized with reference to the patient age categories: ID (system generated); Patient Age Category (Name); Lower Age Limit; Upper Age Limit; Active checkbox; Created On; Created By; Modified On; and Modified By. Similarly, the ability to define patient weight categories is also provided. The following data elements can be utilized with reference to the patient weight categories: ID (system generated); Patient Weight Category (Name); Lower Weight Limit; Upper Weight Limit; Active checkbox; Created On; Created By; Modified On; and Modified By.

The ability to provide dosing by age categories utilizes the following information that is available to the user: Patient Age ID; Patient Age Category; Dose/Dose Range; unit of measure (UOM); unit of measure suffix, e.g., kg, m², day, hr, kg/hr, m²/hr, m²/day, kg/day, min, kg/min, m²/min; Dose Frequency (Admin Rate); Duration of Therapy in hours, Days, Weeks, Doses, Minutes; Created On; Created By; Modified On; and, Modified By. Dosing by weight is also available and will operate similar to the dosing by age categories, but instead is based on the facility defined weight categories.

The user is provided the ability to select the Patient Age Category from the age categories defined in the “Patient Age” reference code table. A history or audit trail is maintained on all table changes.

The healthcare facility is provided the ability to decide whether the recommended drug dosing by patient age should automatically default during order entry, whether the user should get a message recommending the recommended dose, and if override reasons are required when a recommendation is not accepted. These characteristics can be added as checkboxes in a drug file.

For override reasons, if pre-formatted codes are desired then these should be added in the reference code table in a form window and a corresponding table window. A checkbox should be added to designate whether screening should be performed on this drug during order entry.

Another aspect of the present invention involves dosing by lab values. In order to utilize this aspect, the system must be associated with a drug. To set up the dosing by lab values functionality, the following information is provided to the user: Patient Age ID; Patient Age Category; Test/Procedure/Activity ID (Lab test ID); Test/Procedure/Activity Description (Lab test description); Test/Procedure/Activity reference range (Lab test reference range); UOM for the test/procedure/activity (Lab test); Dose/Dose Range; UOM (this should default from the drug file set up); UOM suffix, e.g., kg, m², day, hr, kg/hr, m²/hr, m²/day, kg/day, min, kg/min, m²/min; Created On; Created By; Modified On; and Modified By. The user will be able to select the Patient Age Category from the age categories defined in the “Patient Age” reference code table. Through the above data element selection, the hospital will be able to define age category specific and lab value specific alerts to appear dependent on a specified drug.

During order entry based on the drug selected and the patient's age, one embodiment of the present invention will default the appropriate drug dose/dose range. If the “Display Patient Age Message” checkbox is selected in the drug file, the system will display a message to the user recommending the drug dose according to the patient's age. The user can accept or reject the recommendation. If the recommendation is accepted, the system defaults the drug dose recommended in the dose field on the wizard 52, i.e., Rx dose wizard. If the recommendation is rejected, the system can display, if desired, an override reason window with a pre-defined override reason or reasons in a drop down box. A free text area can also be provided in the override window.

At the time of order entry, the system will also check the patient's lab values associated with the drug being ordered. If there's a match, and the lab value and corresponding dose are outside the range defined for that age category, the system will alert the user of the non-conformity.

If a patient has an order associated with a lab value and a lab result is received, the system will check if the lab value and corresponding dose being ordered is outside the range defined for the specified age category. If so, the system will alert the user of the non-conformity.

The present invention provides a flexible mechanism for creating medical-based logic to provide decision support during patient treatment. Some benefits capable of being attained by the system include: automated message alerting and communication of healthcare facility best practice guidelines; increased patient care safety, efficiency, and outcomes through alerting clinicians of patient parameters; reduced costs associated with more effective patient care management; and, customizable patient care in relation to physician, service, unit, and disease state.

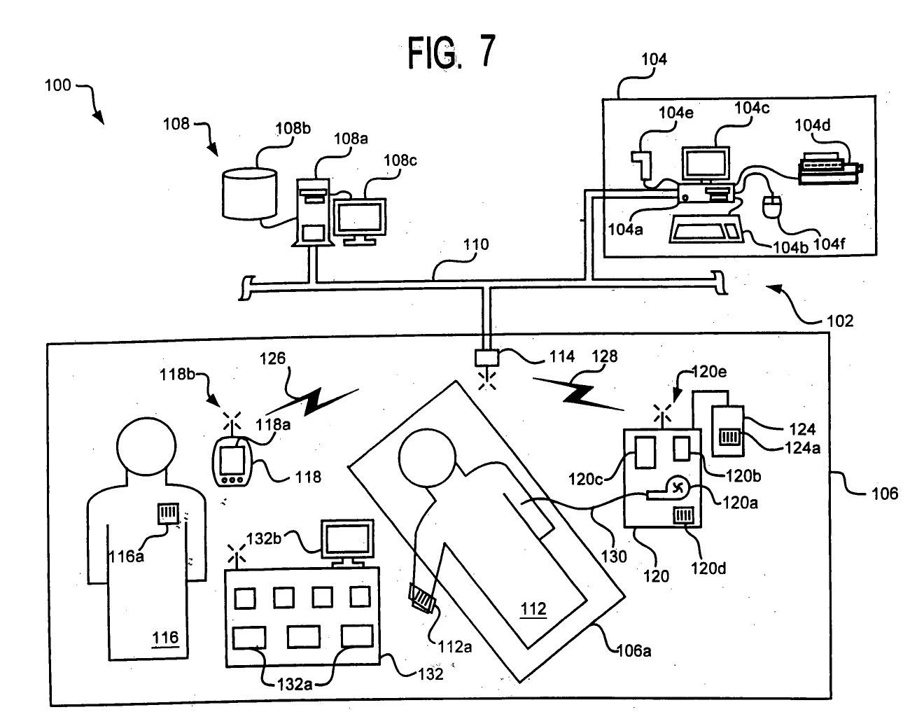

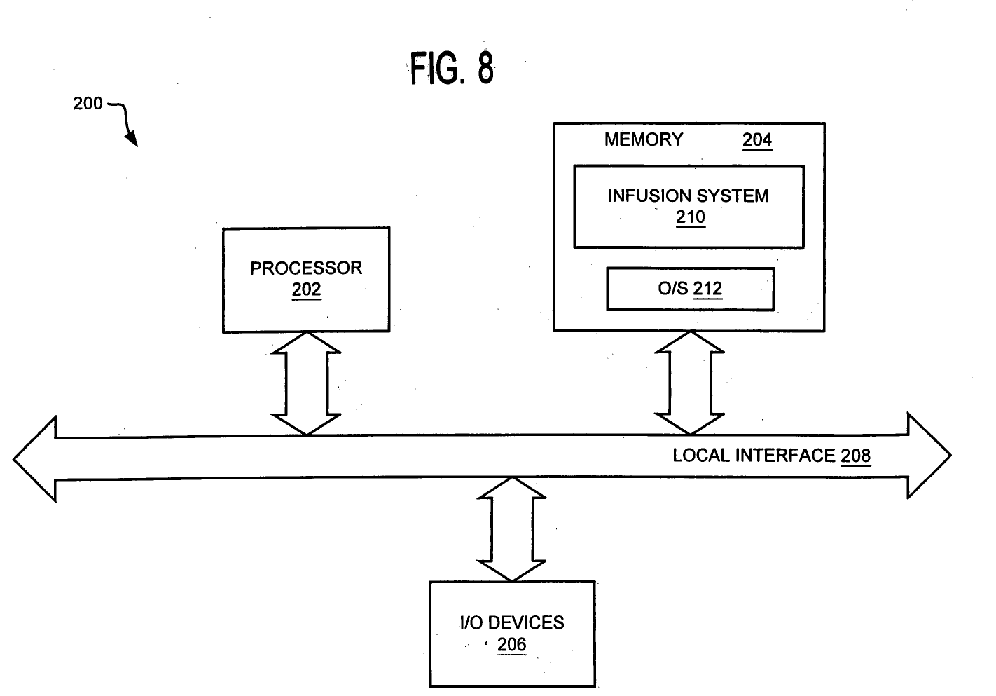

FIG. 7 is a graphical representation of a patient care system. The patient care system 100 includes a pharmacy computer 104, a central system 108, and a treatment location 106, linked by a network 102. Patient care system 100 also includes an infusion system 210 (FIG. 8). Infusion system 210 is a medication system preferably implemented as a computer program, and in particular an application (i.e., a program or group of programs designed for end users), resident on one or more electronic computing devices within the patient care system 100. As described in detail further herein, the infusion system 210 links clinicians, such as physicians, pharmacists, and nurses, in an interdisciplinary approach to patient care.

In an embodiment, clinicians within a healthcare facility have access to infusion alerts, alarms, and messages via a remote wireless device such as a personal digital assistant (PDA) or other computer devices, wireless or hardwired to the network, such as a tablet computer with a bar code reader operably attached, or a laptop computer attached to an IV pole and having a bar code reader operably attached to the computer.

Preferably, the infusion system provides clinicians and other users with options for automating alert event-driven messages. Moreover, healthcare facility administrators and other users can customize the types of automated messaging to appear, via the remote wireless device, by message type or classification, severity of abnormality, and time based reminders. Additionally, the infusion system provides clinicians and other users with the ability to configure audible messages, visual messages, or both.

The messaging provided by the infusion system preferably includes a user configurable rules engine, a scheduler, and interfaces to infusion pump systems. Moreover, it is desired that the results-driven messaging provide clinicians with real-time decision support at the point of care via a workstation, electronic tablet, wireless personal digital assistant, or the like.

Turning back to FIG. 7, patient care system 100 preferably includes a computerized physician order-entry module (CPOE), an inpatient pharmacy module, a wireless nurse charting system, and an electronic patient medical record. It is desired that patient care system 100 provide a comprehensive patient safety solution for the delivery of medication. Within patient care system 100, software modules are provided to link together existing patient care systems using interfaces such as HL7 interfaces that are known to those having ordinary skill in the art. Preferably, the patient care system 100 operates on a variety of computers and personal digital-assistant products to transmit orders, update patient medical records, and access alerts, alarms, and messages.

The computerized physician order-entry module enables physicians to enter medication orders, access alerts, alarms, messages, reminders, vital signs and results. A pharmacy module checks the prescribed drug against documented patient allergies, and for compatibility with other drugs and food. The pharmacy module also provides real-time data for inventory management. A nurse medication-charting module provides clinical information that is immediately available at the bedside, thus ensuring verification of medication and dosage at the point-of-care.

Patient care system 100 integrates drug delivery products with the information required to assist in ensuring safe and effective delivery of medication. The clinical decision support and accompanying alerts, alarms, warnings, and messaging of the patient care system 100 provide a safety net of support for clinicians as they deliver patient care under increasing time and cost pressures. This information is preferably supplied through a wireless network that supplies data in a way that improves clinician workflow, making delivery of care easier.

The infusion system 210 (FIG. 8) within the patent care system 100 provides computerized prescribing and an electronic medical administration record (eMAR). Infusion system 210 puts charting, medication history, inventory tracking, and messaging at the clinician's fingertips. Patient care system 100 combines bar-coding and real-time technology to assist in ensuring that the right patient gets the right medication and the right dosage, at the right time, via the right route. Infusion system 210 provides alerts, alarms, messages, and reminders such as, but not limited to, lab value, out of range, and missed dose. As part of the verification of the right dosage, the system can also provide verification of the settings of an infusion pump.

As explained in detail further herein, the infusion system 210 resides, at least in part, on one or more electronic computing devices such as wireless remote personal digital assistants, workstations, physician order-entry modules, electronic tablets, processor controlled infusion pumps, or the like. The infusion system 210 can be configured to display, via one or more of the electronic computing devices, numerous hospital definable alerts and alarms in varying forms. In an embodiment, time-based alerts are provided to remind clinicians to perform a patient care function such as, but not necessarily limited to, changing an infusion rate. Further, emergency alarms are provided such as, but not necessarily limited to, an infusion being disconnected. Moreover, less urgent message are provided such as, but not necessarily limited to, the infusion being completed or the line being occluded. In addition, the infusion status can be viewed from anywhere within the healthcare facility via one or more of wireless remote personal digital assistants or other electronic computing devices.

In an embodiment, the system 210 provides for the escalation of alarms or alerts that are not indicated as corrected within a predetermined period of time. Conditions that can result in the escalation of an alarm or an alert are preferably defined by the health care facility. Likewise, the time before an alarm or alert escalates can also be defined by the health care facility. Accordingly, predefined alarms or alerts that are not corrected by a clinician within a pre-defined period of time with result in the escalation of the associated alarms or alerts. Thus, the frequency that the clinician is notified by the system of the escalated alarms or alerts is preferably increased, as can be the volume of the audible tones associated therewith. The escalation can also be directed to hospital defined users, workstations, pagers, or the like.

As will be appreciated by those having skill in the art, the infusion system 210 assists in ensuring patient safety by checking the infusion being administered with the patient's order. As explained in detail further herein, a bar coding scheme is used wherein the infusion bag and patient are scanned, the infusion information is displayed on both an electronic computing device and the pump to assist in ensuring that the right infusion is being administered to the right patient and the right time and by the right route and at the right rate. In an embodiment, an alert, audible and visual appears on the electronic device if the above administration “rights” do not match. Moreover, when the clinician sets the infusion pump rate, an audible and visual alert appears on the electronic computing device if the programmed settings do not match the patient's infusion order. In addition, at any time the clinician can, via the electronic device, check the settings of an infusion pump to confirm if the settings match the infusion order as contained within the central database 108b. Also, the clinician can see the time remaining, via the electronic device, or other pump status information.

In an embodiment, the infusion system 210 provides alerts and alarms, via one or more of the electronic computing devices or the like, with differing tones or phrases for fast identification of the severity or urgency of the message. Desirably, conventional infusion pump alerts and alarms can be displayed on the electronic computing devices, such as, but not necessarily limited to, a personal digital assistant, to keep the clinicians informed of the status of the infusions for all assigned patients, thereby saving time in resolving problems and improving workflow safety.

All alarms and alerts are preferably retrievable from a central system database for, inter alia, reporting purposes. The retrievable data can assist a healthcare facility in examining and analyzing how many medication errors were avoided through alarms, alerts, and warnings.

Desirably, the audible alerts and alarms are configured to sound differently according to the severity or urgency associated with the message or issue. Alarms requiring immediate attention sound different from less emergent alerts. Visual text describing the problem is preferably displayed by one or more of the electronic computing devices. In an embodiment, an alert sounds on a personal digital assistant when an infusion is nearing completion or is completed. The personal digital assistant also displays the patient, location, infusion type order text, and the time remaining before the infusion bag is empty. At all times the clinician can access, via the personal digital assistant, the status of infusions and thus react accordingly. In an embodiment, before visiting a patient room, the clinician can view the status of the infusions on the personal digital assistant to determine whether another bag will be needed in the near future. If another infusion bag is needed, the clinician can save time be taking the new bag on the first visit, rather than realizing a new bag is needed after arriving in the patient room. Similarly, the pharmacy can view the status, including time remaining, in order to schedule the mixing and delivery of the next infusion bag.

If desired, and as will be appreciated by those having skill in the art, other alarms and alerts related to the infusion pump can be made available on the electronic computing devices remotely located from the infusion pump. Pertinent information can be displayed on the electronic computing devices, thus saving the nurse time and steps in resolving the problem. As indicated above, when a pump alarms or alerts, the clinician can view patient information, drug order, and alarm or alert message on the personal digital assistant, and gather necessary items before going to the patient room to physically correct the alarm or alert condition.

In an embodiment, the infusion system 210 provides configurable time based alerts for reminding clinicians of scheduled infusion orders. As such, a tapering order to run NS at 200 ml/hr for two hours, then reduce to 50 ml/hr, results in the infusion system 210 alerting the nurse two hours after starting the infusion to reduce the rate. Further, late alerts are provided for informing clinicians when scheduled infusions are past the time tolerance set by the facility. Moreover, time based protocols such as alerts for conducting pains assessments such as after starting an epidural morphine infusion are generated.

Configurable aspects of the infusion system 210 also include the audible alerts emitted by the electronic computing devices, such as personal digital assistants. Preferably, the audible alerts can be configurable by the healthcare facility and within specific units of the healthcare facility to satisfy the unique environments within the healthcare facility.

As indicated previously, a plurality of visual alerts and messages can be displayed by the electronic computing devices, such as personal digital assistants, for indicating the importance or urgency of the message. Desirably, color, flashing, and bold text are display messaging options. Additionally, hyperlinks can be provided when messages are generated. Icons on the displays can also be utilized and emergency messages can be configured to interrupt the handheld electronic device, or the like, to immediately alert the clinician.

As also indicated previously, the infusion system 210 allows a clinician to view all infusions or assigned patients on the electronic computing device, such as a personal digital assistant or the like, thus reducing time spent traveling to and from patient rooms. Moreover, prescription information is displayed on the electronic computing device for verification of the drug amount, diluent, dose, and rate of the infusion. Additionally, real time status of the infusion is viewable for displaying milliliters per hour or the like, duration of the infusion, volume infused, time remaining, and volume yet to be infused. As indicated previously, the status of the infusion can be viewed, and flow rate history, from anywhere within the healthcare facility via the electronic computing devices.

As described in detail further herein, the infusion system 210 calculates ordered doses based on patient weight and displays the appropriate rate to run the infusion. Messages are generated if the infusion is set to run outside of the ordered dose. Moreover, pediatric dosing is available and configured for pediatric units within the healthcare facility.

In an embodiment, the status of primary infusions and secondary infusions, such as piggyback, are displayed by the infusion system 210 on the electronic computing device, such as a personal digital assistant. The clinician can check the volume left to infuse in a piggyback at any time and a message is displayed when the piggyback is completed and the primary infusion has resumed. In addition, messages are sent to the pharmacy to replenish stocks and infusion orders.

If desired, the infusion system 210 allows for the healthcare facility to define system infusion limits for warning a clinician who programs an infusion to run outside of the set range. The warning can be configured to allow clinicians to override the warning or prohibit overrides. As will be appreciated by those having ordinary skill in the art, prohibiting overrides for certain infusions may prevent a patient from inadvertently receiving an overdose.

The infusion system 210 can also provide for displaying reference information pertinent to the needs of each speciality unit within the healthcare facility. Drug information is viewable on the electronic device, such as a personal digital assistant, in addition to speciality unit policies and procedures. Protocols and standard orders can be configured to provide messages based on patient condition. In an embodiment, for example, sliding scale protocols are configured to alert the clinician of a new blood glucose result and to titrate the insulin infusion by a determined number of milliliters based on the sliding scale protocol.

Moreover, through configured rules, messages are sent alerting the nurse of particular infusions as they relate to the patient's condition. In an embodiment, for example, a message is generated when a patient receiving a nephrotoxic infusion has an increase in BUN and Creatinine. Additionally, protocols can be configured to generate messages when certain infusions are titrated. In an embodiment, for example, a message to document a blood pressure can be configured when a clinician titrates a dopamine infusion. Furthermore, hemodynamic monitoring parameters can be linked to infusions to generate messages.

As indicated previously, new infusion orders can be configured to provide messages alerting the clinician of a new order. Messages can be configured as audible and visual such as textual, color alerts, flashing hyperlinks, icons, and the like. Stat orders and discontinue orders can be configured as a high priority message to differentiate them from non-urgent messages.

Preferably, educational messages are generated and configured by the healthcare facility. In an embodiment, for example, an infusion requiring a specific tubing set results in the display of a message informing the clinician. In a further embodiment, for example, an infusion requiring central venous access results in the display of a warning not to infuse in the peripheral vein.

In an embodiment, scheduling messages are generated and displayed on one or more electronic computing devices to remind users to complete the next task. Alerts to change infusion rates at scheduled times are sent to the electronic computing devices, such as in the case of a tapering infusion. Additionally, protocols with time-based alerts can be configured such as, for example blood infusion protocols.

The patient care system 100 also allows computer programs and database tables to perform numerous ordering tasks. For example, schedules of clinician order activities and procedures may be created and provided for access by a user. Access to the orders, however, may be restricted based on the level of authorization provided to a particular care provider. Additionally, orders or components of orders may be created and mapped or linked to structured clinical documentation data elements, groups and/or sets. For example, items (such as medications, supplies or equipment) which may be part of an order may be mapped to structured clinical documentation elements, groups and/or sets. Further, tests (such as glucometer readings, temperature readings, or blood pressure readings) may be mapped to structured clinical documentation data elements, groups and/or sets, or to monitoring parameters. And, procedures (such as changing a dressing or irrigating a wound) may be mapped to structured clinical documentation data elements, groups and/or sets.

Within the system 100, relationships (i.e., parent/child relationships) are created to define how orders that are entered into the system 100, for example by a doctor, translate into the detailed workflow required of the care provider. In such a system, a definition screen may be provided. The definition screen may be invoked by a variety of means, such as a menu item or button, including a “workflow mapping” button.

In one embodiment, the “workflow mapping” option provides the user with a split screen. One portion of the split screen is provided for queries, and another portion of the split screen is provided for results. The query area of the window allows the user to search for previously defined workflow mappings. One means for searching for previous defined workflow mappings is by entering the first few characters of the query (i.e., an orderable item/test/procedure, a monitoring parameter, or a structured clinical documentation data element, group or set). As matches are made for the entered query characters, results are displayed in the results area of the window. In one embodiment, the results area of the window contains a plurality of columns. Examples of various columns include: (a) Orderable Item, (b) Monitoring Parameter, (c) Structured clinical documentation data element, group or set, (d) Active Y/N, (e) Created By, (f) Created Date/Time, (g) Modified By, and (h) Modified Date/Time. The columns allow the user to view the suggested matches to determine if the suggested match is that desired by the user.

One type of relationship between various parameters which may be utilized in order scheduling is a “many to many” relationship. In one embodiment, there is a many to many relationship between one or more parameters, including orderable items, monitoring parameters and structured clinical documentation elements, groups and sets and some cells of the database or spreadsheet. Moreover, because of this relationship, some columns of the database or spreadsheet may be empty (for example, there may be an orderable item that only maps to structured clinical documentation but not to any monitoring parameters or vice versa).

Once results are displayed they may be edited. Editing of the table is done in the following manner: (a) rows in the table may be deleted by selecting them and clicking a delete button based on order status; (b) rows may be added to the table by clicking an Add button to add an empty row; and (c) rows may be edited by the same method of right clicking on individual fields and selecting a new value from the list for that field based on order status. Additionally, when adding rows the Active Y/N check box should be placed in the checked state. By right clicking on the fields, lists of entries available for that field will be supplied, and are available for selection. In the editing mode, rows can be activated or deactivated by toggling the Active check box for the row. If a row is edited or deactivated when there exist active orders using it, a warning is produced and the user can view all such orders before deciding whether or not to proceed.

The system 100 also includes a history feature, which may be a history button. The history feature provides a complete audit trail for the mapping table.

Scheduling orders may also be created by selecting an orderable item from the table. As explained above, examples of orderable items include, but are not limited to, drugs, supplies and equipment. Additionally, as explained above, searches or queries for orderable items may be filtered using a few of the first letters of the name of the orderable item. The user entering the order also selects a schedule using the same scheduling tables that apply to medication orders. The user may also enter additional instructions that apply to the order and designate them “As Required.”

The system 100 also allows for creating standard orders and standard order sets. Standard orders and standard order sets are created in a similar manner as explained above. The order sets can define dependencies between orders to provide the interdependency mentioned previously. For example, an order set may define a scheduled order for a pain assessment, and then a medication order for morphine that is conditional on the assessment, and finally a post-administration order for another pain assessment 30 minutes after the administration, if it occurred.

Order scheduling is typically operated on a wireless device such as a Personal Digital Assistant, tablet, or laptop computer. In one embodiment, the system 100 provides an electronic To Do list showing all orders scheduled or conditionally required during a time period such as a nursing shift. The system 100 also includes highlighting on the list of patients to identify those activities that are ordered during the current time period. When a patient is selected, all activities that are scheduled or conditionally required for the time period are identified and can be documented. The activity is organized into different tabs for easy viewing.

Turning to FIG. 7, and as indicated above, patient care system 100 allows medication ordering, dispensing, and administration to take place at the patient's bedside. Physicians can order simple and complex prescriptions, intravenous therapy and total parental nutrition therapy (TPN) using a wireless handheld device, touchscreen table, laptop computer, or the like. Infusion system 210 checks for drug interactions and other possible errors as well as correct dosage. Infusion system 210 then transmits this data in real-time to the patient care facility or local pharmacy, hospital nursing unit, home care unit, and/or clinic. The infusion system can also notify the clinician of the correct route of the administration.

The clinician can access a medical records database using a handheld scanning device. In an embodiment, the clinician scans the bar coded medication and the patient's bar coded bracelet to confirm the presence of the right medication, dosage, and time before administering any drugs. Infusion system 210 updates medical and administrative records, thereby eliminating most, if not all, time-consuming paperwork. Thus, infusion system 210 can reduce costs and improves efficiency while possibly saving lives. Patient care system 100 can include access-controlled mobile and stationary medication and supply depots, including electronic patient medical records and computerized prescribing, providing complete preparation and inventory management from the point of care to the pharmacy.

As mentioned previously, FIG. 7 is a graphical representation of patient care system 100. The patient care system 100 includes a pharmacy computer 104, a central system 108, and a treatment location 106, linked by a network 102. In an embodiment, the pharmacy computer 104 includes a processing unit 104a, a keyboard 104b, a video display 104c, a printer 104d, a bar code reader 104e, and a mouse 104f. Although not shown in FIG. 7, the patient care system 100 can also include subsystems for hospital administration, nursing stations, a clinical information subsystem, a hospital information subsystem, an Admissions Discharge and Transfer (ADT) subsystem, a billing subsystem, and/or other subsystems typically included in conventional patient care systems.

In an embodiment, the central system 108 includes a central servicing unit 108a, a database 108b, a video display 108c, input/output components, and other conventional hardware components known to those having ordinary skill in the art. The network 102 preferably includes a cable communication system 110 portion and a wireless communication system portion. The cable communication system 110 can be, but is not limited to, an Ethernet cabling system, and a thin net system.

In an embodiment, the treatment location 106 can include a treatment bed 106a, an infusion pump 120, and medical treatment cart 132. In FIG. 7, a clinician 116 and a patient 112 are shown in the treatment location 106. Medication 124 can be of a type that is administered using an infusion pump 120. Medication 124 can also be of a type that is administered without using an infusion pump. The medication can be stored in medication storage areas 132a of medical treatment cart 132. The clinician 116 uses a digital assistant 118 in the process of administering medication 124 to the patient 112.

In an embodiment, the clinician 116 uses the digital assistant 118 in the course of treating patient 112 to communicate with the cable communication system 110 of the network 102 via a first wireless communication path 126. The infusion pump 120 has the ability to communicate with the cable communication system 110 via a second wireless communication path 128. The medication cart 124 also has the ability to communicate via a wireless communication path (not shown in FIG. 7). A wireless transceiver 114 interfaces with the cable communication system 110. The wireless communication system portion of the network can employ technology such as, but not limited to, known to those having ordinary skill in the art such as IEEE 802.11b “Wireless Ethernet,” a local area network, wireless local area networks, a network having a tree topography, a network having a ring topography, wireless internet point of presence systems, an Ethernet, the Internet, radio communications, infrared, fiber optic, and telephone. Though shown in FIG. 7 as a wireless communication system, the communication paths can alternatively be hardwired communication paths.

In the patient care system 100, a physician can order medication 124 for patient 112. In an embodiment, the order can originate with a clinician 116 at the treatment location 106. The physician and/or clinician 116 can use a computerized physician order entry system (CPOE), the medical cart 132, or a like device, to order the medication 124 for the patient 112. Those having ordinary skill in the art are familiar with conventional computerized physician order entry systems. Despite its name, any clinician 116 can use the computerized physician order entry system. If the medication 124 is efficient to administer through infusion pump 120, the infusion order includes information for generating operating parameters for the infusion pump 120. The operating parameters are the information and/or instruction set necessary to program infusion pump 120 to operate in accordance with the infusion order.

The infusion order can be entered in a variety of locations including the pharmacy, the nursing center, the nursing floor, and treatment location 106. When the order is entered in the pharmacy, it can be entered in the pharmacy computer 104 via input/output devices such as the keyboard 104b, the mouse 104f, a touch screen display, the CPOE system and/or the medical treatment cart 132. The processing unit 104a is able to transform a manually-entered order into computer readable data. Devices such as the CPOE can transform an order into computer readable data prior to introduction to the processing unit 104a. The operating parameters are then printed in a bar code format by the printer 104d on a medication label 124a. The medication label 124a is then affixed to a medication 124 container. Next, the medication 124 container is transported to the treatment location 106 or remotely from the healthcare facility. The medication 124 can then be administered to the patient 112 in a variety of ways known in the art including orally and through an infusion pump 120. If the medication 124 is administered orally, the clinician 116 can communicate via the digital assistant 118 and/or the medical cart 132. The medical cart 132 is computerized and generally has a keyboard (not shown), a display 132b, and other input/output devices such as a bar code scanner (not shown).

As will be appreciated by those having ordinary skill in the art, the infusion bag can also be premixed, wherein a non-patient specific bar code is attached to the bag identifying the medication 124. Moreover, the infusion bag can be mixed in the pharmacy or on the floor, wherein a patient specific bar code is attached to the bag that identifies the medication 124 and, if desired, when the medication is to be administered to the patient.

At the treatment location, the medication 124 can be mounted on the infusion pump 120 with an intravenous (IV) line 130 running from the infusion pump 120 to the patient 112. The infusion pump 120 can include a pumping unit 120a, a keypad 120b, a display 120c, an infusion pump ID 120d, and an antenna 120e. Prior art infusion pumps can be provided with a wireless adaptor (not shown) in order to fully implement the system 100. The wireless adaptor can have its own battery if necessary to avoid reducing the battery life of prior art infusion pumps. The wireless adaptor can also use intelligent data management such as, but not limited to, store-and-forward data management and data compression to minimize power consumption and network traffic. The wireless adaptor can also include the ability to communicate with the digital assistant 118 even when the network 102 is not functioning.

In an embodiment, the patient care system 100 can include a variety of identifiers such as, but not limited to, personnel, equipment, and medication identifiers. In FIG. 7, the clinician 116 can have a clinician badge 116a identifier, the patient 112 can have a wristband 112a identifier, the infusion pump 120 can have an infusion pump ID 120d identifier, and the medication 124 can have a medication label 124a identifier. Clinician badge 116a, wristband 112a, infusion pump ID 120d, and medication label 124a include information to identify the personnel, equipment, or medication they are associated with. The identifiers can also have additional information. For example, the medication label 124a can include information regarding the intended recipient of the medication 124, operating parameters for infusion pump 120, and information regarding the lot number and expiration of medication 124. The information included in the identifiers can be printed, but is preferably in a device readable format such as, but not limited to, an optical readable device format such as a bar code, a radio frequency (RF) device readable format such as an RFID, an iButton, a smart card, and a laser readable format. The digital assistant 118 can include a display 118a and have the ability to read the identifiers including biometric information such as a fingerprint.

The wristband 112a is typically placed on the patient 112 as the patient 112 enters a medical care facility. The wristband 112a includes a patient identifier. The patient identifier can include printed information to identify the patient and additional information such as a treating physician's name(s). The patient identifier for patient 112 can include information such as, but not limited to, the patient's name, age, social security number, the patient's blood type, address, allergies, a hospital ID number, and the name of a patient's relative. In an embodiment, the patient identifier can contain a unique reference code or password for the patient, which is also stored in the central database for cross referencing, if needed or desired.

FIG. 8 is a block diagram of a computer 200 representative of the pharmacy computer 104, the central system 108, the CPOE, the digital assistant 118 of FIG. 7, and/or a computer included in any number of other subsystems that communicate via the network 102 such as the medication treatment cart 132. As indicated previously, the computer 200 includes an infusion system 210, or a portion of infusion system 210, for use within the patent care system 100. The infusion system as described in reference to FIG. 8 is preferably a computer program. However, the infusion system can be practiced in whole or in part as a method and system other than as a computer program.

A critical concern in the art is that the right medication is administered to the right patient. Therefore, infusion system 210 includes features to assist in assuring that the right medication is administered to the right patient in an efficient manner. Infusion system 210 can be implemented in software, firmware, hardware, or a combination thereof. In one mode, infusion system 210 is implemented in software, as an executable program, and is executed by one or more special or general purpose digital computer(s), such as a personal computer (PC; IBM-compatible, Apple-compatible, or otherwise), personal digital assistant, workstation, minicomputer, or mainframe computer. An example of a general-purpose computer that can implement the infusion system 210 of the present invention is shown in FIG. 8. The infusion system 210 can reside in, or have various portions residing in, any computer such as, but not limited to, pharmacy computer 104, central system 108, medication treatment cart 132, and digital assistant 118. Therefore, the computer 200 of FIG. 8 is representative of any computer in which the infusion system 210 resides or partially resides.

Generally, in terms of hardware architecture, as shown in FIG. 8, the computer 200 includes a processor 202, memory 204, and one or more input and/or output (I/O) devices 206 (or peripherals) that are communicatively coupled via a local interface 208. The local interface 208 can be, for example, but not limited to, one or more buses or other wired or wireless connections, as is known in the art. The local interface 208 can have additional elements, which are omitted for simplicity, such as controllers, buffers (caches), drivers, repeaters, and receivers, to enable communications. Further, the local interface can include address, control, and/or data connections to enable appropriate communications among the other computer components.

Processor 202 is a hardware device for executing software, particularly software stored in memory 204. Processor 202 can be any custom made or commercially available processor, a central processing unit (CPU), an auxiliary processor among several processors associated with the computer 200, a semiconductor-based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80×86 or Pentium series microprocessor from Intel Corporation, a PowerPC microprocessor from IBM, a Sparc microprocessorfrom Sun Microsystems, Inc., or a 68xxx series microprocessor from Motorola Corporation. Processor 202 can also represent a distributed processing architecture such as, but not limited to, SQL, Smalltalk, APL, KLisp, Snobol, Developer 200, MUMPS/Magic.

Memory 204 can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory 204 can incorporate electronic, magnetic, optical, and/or other types of storage media. Memory 204 can have a distributed architecture where various components are situated remote from one another, but are still accessed by processor 202.

The software in memory 204 can include one or more separate programs. The separate programs comprise ordered listings of executable instructions for implementing logical functions. In FIG. 8, the software in memory 204 includes the infusion system 210 in accordance with the present invention and a suitable operating system (O/S) 212. A non-exhaustive list of examples of suitable commercially available operating systems 212 is as follows: (a) a Windows operating system available from Microsoft Corporation; (b) a Netware operating system available from Novell, Inc.; (c) a Macintosh operating system available from Apple Computer, Inc.; (d) a UNIX operating system, which is available for purchase from many vendors, such as the Hewlett-Packard Company, Sun Microsystems, Inc., and AT&T Corporation; (e) a LINUX operating system, which is freeware that is readily available on the Internet; (f) a run time Vxworks operating system from WindRiver Systems, Inc.; or (g) an appliance-based operating system, such as that implemented in handheld computers or personal digital assistants (PDAs) (e.g., PalmOS available from Palm Computing, Inc., and Windows CE available from Microsoft Corporation). Operating system 212 essentially controls the execution of other computer programs, such as infusion system 210, and provides scheduling, input-output control, file and data management, memory management, and communication control and related services.

Infusion system 210 can be a source program, executable program (object code), script, or any other entity comprising a set of instructions to be performed. When a source program, the program is translated via a compiler, assembler, interpreter, or the like, that may or may not be included within the memory 204, so as to operate properly in connection with the O/S 212. Furthermore, the infusion system 210 can be written as (a) an object oriented programming language, which has classes of data and methods, or (b) a procedural programming language, which has routines, subroutines, and/or functions, for example, but not limited to, C, C++, Pascal, Basic, Fortran, Cobol, Perl, Java, and Ada. In one embodiment, the system program 210 is written in C++. In other embodiments, the infusion system 210 is created using Power Builder. The I/O devices 206 can include input devices, for example, but not limited to, a keyboard, mouse, scanner, microphone, touch screens, interfaces for various medical devices, bar code readers, stylus, laser readers, radio-frequency device readers, etc. Furthermore, the I/O devices 206 can also include output devices, for example, but not limited to, a printer, bar code printers, displays, etc. The I/O devices 206 can further include devices that communicate as both inputs and outputs, for instance, but not limited to, a modulator/demodulator (modem; for accessing another device, system, or network), a radio frequency (RF) or other transceiver, a telephonic interface, a bridge, a router, etc.

If the computer 200 is a PC, workstation, personal digital assistant, or the like, the software in the memory 204 can further include a basic input output system (BIOS) (not shown in FIG. 2). The BIOS is a set of essential software routines that initialize and test hardware at startup, start the O/S 212, and support the transfer of data among the hardware devices. The BIOS is stored in ROM so that the BIOS can be executed when the computer 200 is activated.

When the computer 200 is in operation, processor 202 is configured to execute software stored within memory 204, to communicate data to and from memory 204, and to generally control operations of the computer 200 pursuant to the software. The infusion system 210 and the O/S 212, in whole or in part, but typically the latter, are read by processor 202, perhaps buffered within the processor 202, and then executed.

When the infusion system 210 is implemented in software, as is shown in FIG. 8, the infusion system 210 program can be stored on any computer readable medium for use by or in connection with any computer related system or method. As used herein, a computer readable medium is an electronic, magnetic, optical, or other physical device or means that can contain or store a computer program for use by or in connection with a computer related system or method. The infusion system 210 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

In another embodiment, where the infusion system 210 is implemented in hardware, the infusion system 210 can be implemented with any, or a combination of, the following technologies, that are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

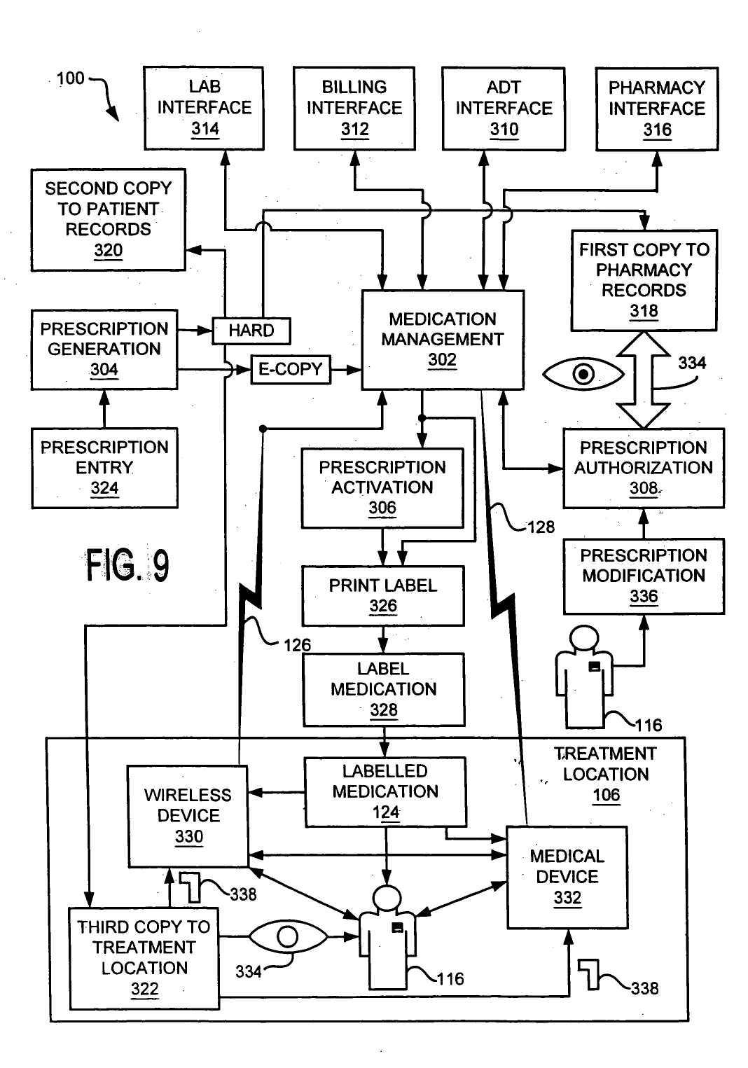

Any process descriptions or blocks in figures, such as FIGS. 9-16, are to be understood as representing modules, segments, or portions of hardware, software, or the like, that can include one or more executable instructions for implementing specific logical functions or steps in the process, and alternate implementations are included within the scope of the embodiments of the present invention in which functions can be executed out of order from that shown or discussed, including substantially concurrently or in reverse order, depending on the functionality involved, as would be understood by those having ordinary skill in the art.

FIG. 9 is a first block diagram showing functional components of the patient care system 100 of FIG. 7. As shown in FIG. 9, the patient care system 100 can be practiced as a modular system where the modules represent various functions of the patient care system, including the infusion system 210 (FIG. 8). The flexibility of the patient care system 100 and the infusion system can be enhanced when the systems are practiced as modular systems. The modules of the infusion system 210 (FIG. 8) can be included in various portions of the patient care system 100. In an embodiment, the patient care system functional components can include, inter alia, a medication management module 302, a prescription generation module 304, a prescription activation module 306, and a prescription authorization module 308.

The medication management module 302 can coordinate the functions of the other modules in the patient care system 100 that are involved in the administration of medical treatment. The medication management module 302 generally coordinates with other portions of the patient care system 100. The medication module 302 can include sub-modules for operating and/or interfacing with a CPOE, for operating and/or communicating with point-of-care modules, and for operating and/or communicating with medical treatment comparison modules. In FIG. 9, an admissions, discharge, and transfer (ADT) interface 310, a billing interface 312, a lab interface 314, and a pharmacy interface 316 are shown. The ADT interface 310 is used to capture information such as the patient's demographics, size, weight, and allergies. Pharmacy interface 316 imports orders from the pharmacy. The pharmacy interface 316 can be an HL7 type of interface that interfaces with other systems for entering orders, such as a CPOE. This ability reduces the necessity for entering data into the patient care system 100 more than once. The pharmacy interface 316 can be configured to communicate with commercially available systems such as, but not limited to Cerner, HBOC, Meditech, SMS, Phamous, and the like. Various other interfaces are also known to those having ordinary skill in the art but are not shown in FIG. 9.

The medication management module 302 can have additional features such as the ability to check for adverse reactions due to drug-to-drug incompatibility, duplicate drug administration, drug allergies, drug dosage limitations, drug frequency limitations, drug duration limitations, and drug disease contraindications. Food and alcohol interactions can also be noted. Drug limitations can include limitations such as, but not limited to, limitations associated with adults, children, infants, newborns, premature births, geriatric adults, age groupings, weight groupings, height groupings, and body surface area. In an embodiment, the medication management module 302 prevents the entry of the same prescription for the same patient from two different sources within the patient care system 100.

The medication management module 302 can also include the ability to generate reports. The reports include, but are not limited to, end-of-shift, titration information, patient event lists, infusion history, pump performance history, pump location history, and pump maintenance history. The end-of shift report can include the pump channel, start time, end time, primary infusion, piggyback infusion, medication, dose, rate, pump status, volume infused, volume remaining, time remaining, and the last time cleared. The infusion history report includes medications and volume infused.

The medication management module 302 can also include a medical equipment status database. The medical equipment status database includes data indicating the location of a medical device 332 within the patient care system 100. The medical equipment status database can also include data indicating the past performance of a medical device 332. The medical equipment status database can also include data indicating the maintenance schedule and/or history of a medical device 332.

Infusion prescriptions are entered in prescription entry 324. Prescriptions can include prescriptions such as, but not limited to, single dose infusions, intermittent infusions, continuous infusions, sequencing, titrating, and alternating types. Infusion prescriptions can also include total parenteral nutritional admixtures (TPN), chemotherapy continuous infusion, piggybacks, large volume parenterals, and other infusion prescriptions. The patient care system 100 can function without end dates for orders. The patient care system 100 uses a continuous schedule generator that looks ahead a predefined time period and generates a schedule for admixture filling for the time period. The predefined time period can be defined at the patient care system 100 level or at subsystem levels such as the clinical discipline level and an organizational level. The predefined time periods can be adjustable by the clinician 116 entering the order. The schedule can be automatically extendable as long as the order is active in the patient care system 100.

The prescription generation module 304 generates hard prescriptions and electronic (E-copy) prescriptions. Hard prescriptions are generally produced in triplicate in medical facilities. A first hard copy 318 is generally sent to the is pharmacy, a second hard copy 320 is generally kept for the patient's records, and third hard copy 322 is sent to treatment location 106. An electronic prescription is sent to the medication management module 302.

Prescription generation module 304 can include confirming operating parameters. The operating parameters can be based on information from prescription entry module 324. Prescription generation 304 can occur anywhere in the patient care system 100 such as, but not limited to, the pharmacy, the treatment location 106, and a nursing center.

A computerized physician order entry (CPOE) system or the like can be employed to carry out some or all of the functions of the prescription generation module 304. Clinicians 116 can enter data in a variety of manners such as, but not limited to, using a tablet wireless computer, personal digital assistant, treatment cart 132, and a workstation. The medication management module 302 can interface with more than one prescription generation module 304. The medication management module can receive orders from anywhere within the patient care system 100.

The pharmacy computer 104 is able to access the electronic copy from the medication management module 302. The prescription activation module 306 is a computer assisted system for coordinating the filling and labeling of prescriptions. The filling of the prescription and the creation or location of medication 124 from stock is handled by the prescription activation module 306. In an embodiment, the filling process results in the creation of the medication label 124, as opposed to the prescription activation process.

The patient care system 100 can bypass the prescription activation module 306. This can occur if the ordering clinician 116, such as the patient's physician, has the authority to immediately activate an order. If the order is immediately activated, the medication management module 302 can go directly to filling and thus, the prescription labeling module 326.

In block 326, the patient care system 100 prints the medication label 124. The prescription can be printed remotely and will often be printed by the pharmacy printer 104d. After block 326, the patient care system goes to block 328. In block 328, the medication label 124a is attached to the medication 124. The pharmacist generally provides a visual verification 334 that the medication label 124a matches the first hard copy 318 of the prescription. FIG. 9 shows that a visual verification 334 is also associated with prescription authorization module 308. The medication 124 can then be transported from the pharmacy to the treatment location 106. A portable medical treatment cart 132 can be used for a portion of the route from the pharmacy to the treatment location 106.

The medication label 124a can include information for preparing the infusion bag. If not generated within patient care system 100, medication label 124a can be provided by a bulk medication supplier. If provided by a bulk medication supplier, the patient care system 100 gathers the information from the medication label 124a. In addition, the patient care system 100 can add information, such as a patient identifier, to the medication label 124a.

The medication labeling module 328 places the medication label 124 on the medication 124. This can be accomplished manually. This can also be accomplished using an automatic prescription filling and packaging system (not shown). If an automatic filling and packaging system is used, medication labeling module 328 provides data for coordination of the labeling of the medication 124 to the filling and packaging system.

At the treatment location 106, the clinician 116 uses a wireless device 330, such as digital assistant 118 and/or medical treatment cart 132, to verify and administer medication 124 to the patient 112. Wireless device 330 communicates with the medication management module 302 via a communication path, such as first communication path 126.

Clinician 116 can identify his/herself by scanning badge 116a, identifies the patient 112 by scanning wristband 112a, identifies the medication 124 by scanning medication label 124a, and identifies the medical device 332, such as infusion pump 120, by scanning label 120d. Clinician 116 can also identify his/herself by providing a fingerprint and/or password. The medical device 332 can be a medical device capable of two-way communication with the medication management module 302. Alternatively, the medical device 332 can only be capable of providing information to the medication management module 302. The infusion program 210 assists the clinician 116 in administering and verifying the medical treatment. The infusion program 210 can include downloading of operating parameters to the medical device 332. Clinician 116 can provide a visual verification to confirm the third copy 322 and/or the MAR matches the labeled medication 124. Scanner 338 can be used to enter machine readable information from the third copy 322 to the wireless device 330 and the medical device 332.

The patient care system 100 can make adjustments and modifications to infusion orders. Among other modules that can include the ability to make infusion adjustments are prescription entry 324, prescription activation 306, prescription authorization 308, and prescription modification module 336. Clinician 116 accesses the prescription modification module 336 in order to make adjustments to an order. The clinician 116 can access the prescription modification module 336 throughout the patient care system 100. However, one very useful location for clinician 116 to access the prescription modification module 336 is at treatment location 106.

In prescription authorization module 308, the patient care system 100 determines whether the clinician 116 has the authority to independently modify an infusion order. The clinician 116 can be recognized by the patient care system 100 as having the authority to independently modify certain portions of the order. If the clinician 116 does not have the authority to independently modify the order, a pharmacist or physician can be requested to approve the modification entered by the clinician 116.

In one implementation of patient care system 100, an order is entered in pharmacy computer 104. The order includes a first patient identifier and an operating parameter. The pharmacy computer 104 generates a medication label 124a that is affixed to the medication bag or container. The medication 124 is sent to a treatment location 106. At treatment location 106, clinician 116 reads the clinician's badge 116a, patient's wristband 112a, and medication label 124a with a digital assistant 118. The digital assistant 118 reports, based on a determination made by the central system 108, whether medication label 124a and wristband 112a correspond to the same patient 112. The system 400 then sends the medication identifier to the pharmacy computer 104. The pharmacy computer 104 confirms the medication label 124a identifies the same patient as the order and sends the operating parameter to an infusion pump. The operating parameter can be sent directly to the infusion pump 120. The operating parameter is then used to program the infusion pump to administer the medication 124 to the patient 112.

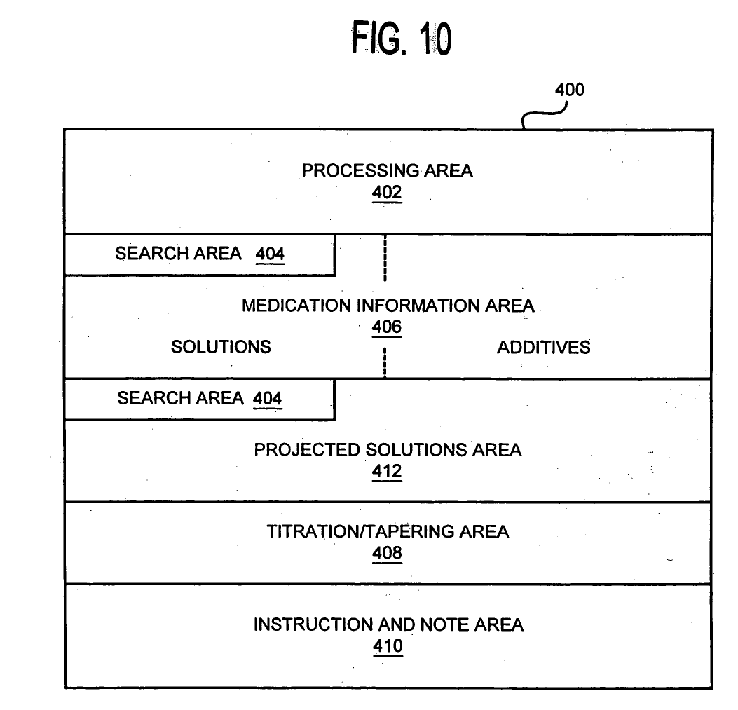

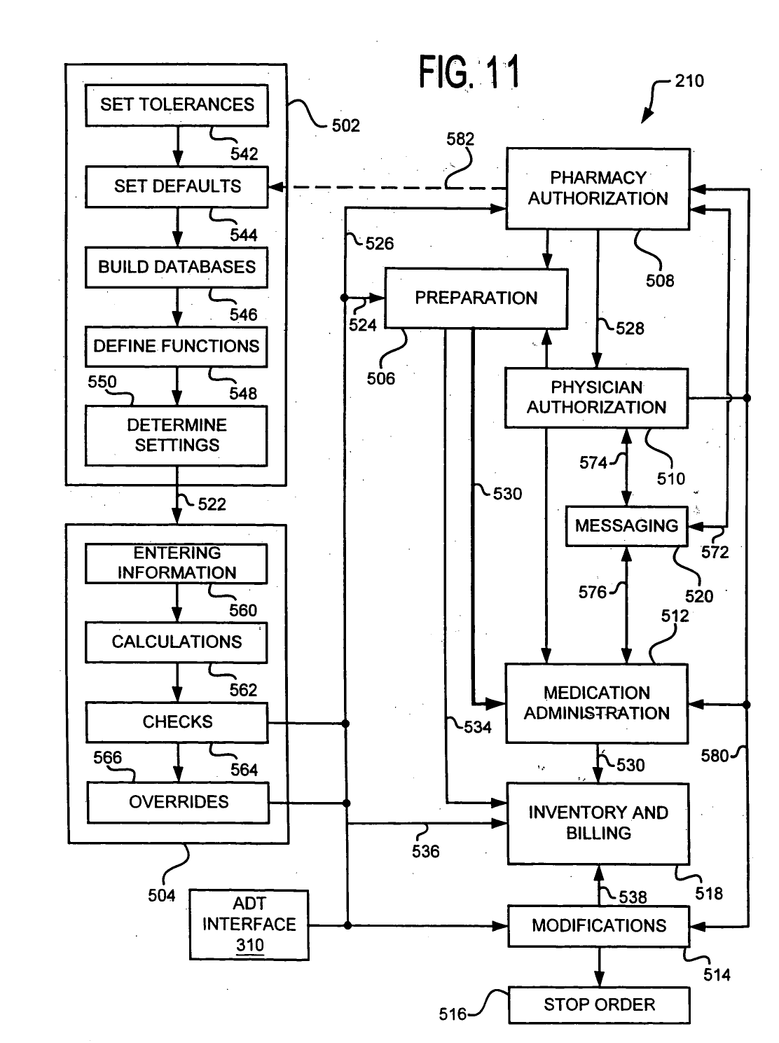

FIG. 10 is an exemplar block diagram of a computer screen 400 that is useful in implementing various functions of the infusion system 210 (FIG. 8). In addition to other functions, the computer screen 400 can be used to enter new infusion orders, to modify existing infusion orders, and to stop infusion orders. Computer screen 400 preferably includes a processing area 402, search areas 404, a medication information area 406, a titration/Tapering criteria area 408, an instruction and note area 410, and a projected solution ingredient area 412. Infusion medication order types include single dose, intermittent, continuous, sequencing, and alternating. Computer screen 400 can be used with digital assistant 118, pharmacy computer 104, infusion pump 120, a CPOE system, and medical treatment cart 132. Computer screen 400 is generally designed to have the look-and-feel of clinician accessible computer screens throughout the patient care system 100 of FIG. 7. The functions of computer screen 400 are partially accomplished with database linkage techniques familiar to those having ordinary skill in the art such as, but not limited to, hyperlinks, definition boxes, and dropdown menus. Computer screen 400 is one example of the type utilized for the ordering schedule aspect of the present invention.

The processing area 402 includes the ability to trigger the creation of an infusion order, a save of an infusion order, the modification of an infusion order, and a cancellation of an infusion order. Clinician 116 can customize the computer screen 400 to provide the clinician's 116 preferred order entry procedures. The processing area 402 includes a status indicator for orders. The processing area 402 also includes an area for indicating whether a PRN order (“as required” or “when needed” order) can be placed by clinician 116. The processing area 402 further includes the ability to display and adjust medical device 332 operating parameters, infusion order route, infusion line, infusion administration site, infusion order start time, infusion medication order type, infusion flow rate tolerance, infusion flow rate, infusion duration, area of preparation (such as pharmacy or a remote site). The processing area 402 can also include an area for linking medical orders to other medical orders such as, linking a physician's infusion order to another medical order entered by another clinician 116. The processing area 402 can include a trigger for displaying data in other areas of the computer screen 400 such as, but not limited to the projected solutions area 412.

Search areas 404 allow for searching for medications, solutions and/or additives for infusion orders. Default diluents can be provided for orders. If a default dosage for a medication is defined in the patient care system 100, the default dosage automatically appears with the search result that includes the medication. A search from search area 404, can result in the display of the medication name, the route of administration, the cost, the package size, the dosage form, the generic name, whether the medication is a narcotic, whether the medication is controlled, whether formulary, and whether the medication is manufactured.

Medication information area 406 can be used to define infusion order additives and solutions. Medication information area 406 can include separate additive areas and solution areas. The solution area can include a label “Solution/Diluent”. The patient care system 100 may use a medication 124 database, a solutions database, and an additive database to populate the medication information area 406 with medications 124, solutions, and additives. Substances identified in one database may also be identified in other databases. The databases may be linked to provide default values for combinations of the medications 124 and solutions.

Titration/tapering criteria area 408 generally applies to continuous infusion orders. Titration defines certain parameters of an order such as dosage and/or flow rate. Dose and flow rate can be entered as an absolute. Also, mathematical symbols such as, but not limited to, greater than “>”, less than “<”, and equal “=”, can be used alone or in combination to enter information in titration/tapering criteria area 408. A calendar can also be used to enter data in titration/tapering criteria area 408. Dosage and flow rate can also be entered as an acceptable range. Titration/tapering criteria area 408 can be hidden when non-continuous infusion orders are entered and/or modified. The titration criteria can include values of various parameters related to patient condition such as, but not limited to, various lab results, vital signs, ability to take fluids orally, fluid input and output, and the like.

Instruction and note area 410 includes the ability to save information such as physician notes regarding a patient 112 and/or an infusion order. The instruction and note area 410 can include a display and lookup area for identifying clinicians 116 that are responsible for the patient 112, such as the patient's physician.

The projected solutions area 412 displays solution schedules and related ingredients based on the current state of the order being processed for patient 112. The time period projected can be a patient care system 100 default. The time period can also be adjustable by the clinician 116. The projected solutions area 412 can include an adjustable display indicating the time period projected by the patient care system 100. The data displayed in the projected solutions area is generally saved when an order save is triggered in the processing area 402. The projected solutions area 412 can include the ability to look back over a period of time while modifying a previously entered order. This allows the clinician 116 to view solutions that may have already been prepared according to the unmodified infusion order.

FIG. 11 is a block diagram showing functional components of the infusion system 210 of FIG. 8. The functional components include blocks for setting system parameters 502, infusion order creation 504, infusion order preparation 506, medication administration 512, infusion order modifications 514, and messaging 520. FIG. 11 also includes blocks for pharmacy authorization 508, physician authorization 510, stop orders 516, and inventory and billing 518. FIG. 11 presents one description of the infusion system. However, FIG. 11 does not define a required series of steps for implementing the infusion system. One of the benefits of the infusion system is that a clinician 116 can access and enter information from a large number of locations, both physical and functional, within the patient care system 100. For example, an infusion order can be created by a physician using a CPOE, by a pharmacist using pharmacy computer 106, by a clinician 116 using digital assistant 118, and by a clinician using medication treatment cart 132. Moreover, vitals, lab results, and other records of patients can be checked from a large number of locations within the health care facility including, for instance, the inpatient pharmacy. Accordingly, a user within the inpatient pharmacy 104 (FIG. 7) can view, from a computing device 104c, the wards within the health care facility. Upon selection of a ward by the user, a patient list is provided wherein the user can select a patient and associated records for display on the computing device. Alternatively, the user can enter all or part of the patient's name into the computing device, whereby the records associated with the patient are provided by the computing device for selection by the user. Upon selection, the record(s) is displayed.

In an embodiment, FIG. 11 can be viewed as first preparing the patient care system 100 for receiving infusion orders—setting system parameters 502; second, creating the infusion order—infusion order creation 504; third, preparing the infusion order—preparation 506; fourth, authorizing the infusion order—pharmacy and physician authorization 508 and 510; fifth, administering the infusion order—medication administration 512; sixth, accounting for and replenishing the inventory used to prepare the infusion order and billing the patient for the infusion order—inventory and billing 518; seventh, modifying the infusion order—modifications 514.; and eight, providing messages to various personnel and sub-systems regarding the progress of the infusion order, infusion, messages for assisting in ensuring that the right medication is efficiently prepared and provided to the right patient, in the right dose and at the right time, or the like—messages 520. Modifications 514 can include stopping the order—stop order 516—based on information provided by the transfer interface 310.

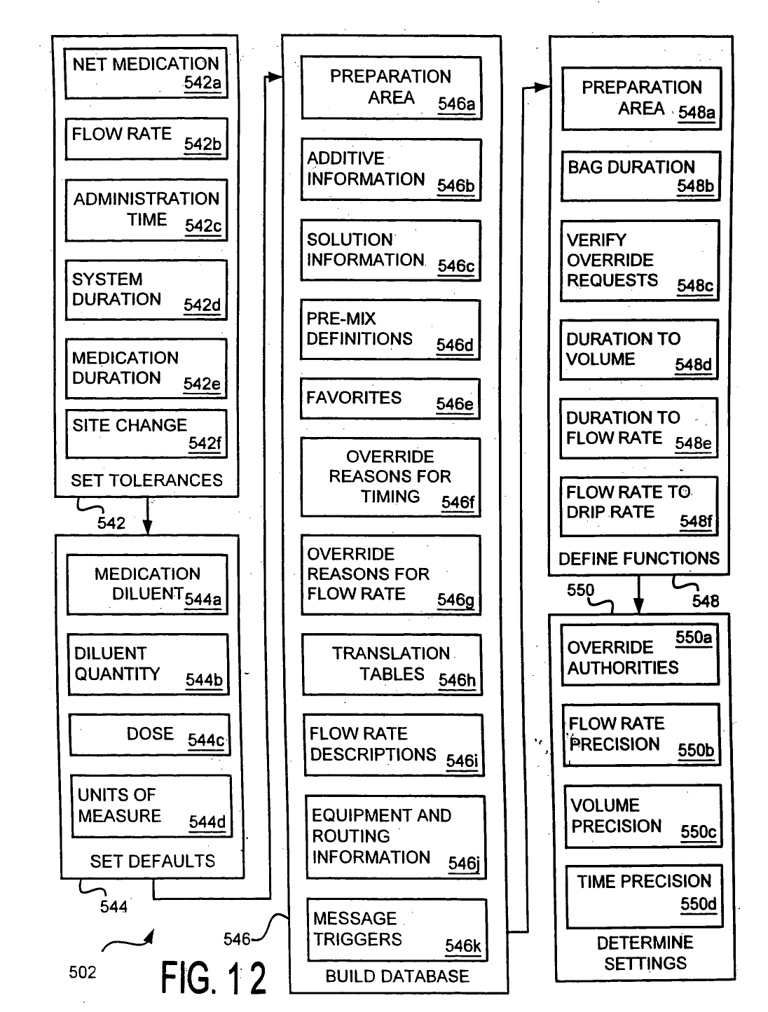

Setting system parameters 502 includes functional blocks that prepare the infusion system 210 to create and process infusion orders. Setting system parameters 502 include, but is not limited to, setting tolerances 542, setting defaults 544, building databases 546, defining functions 548, and determining system settings 550. Setting system parameters 502 is further described below in reference to FIG. 12.

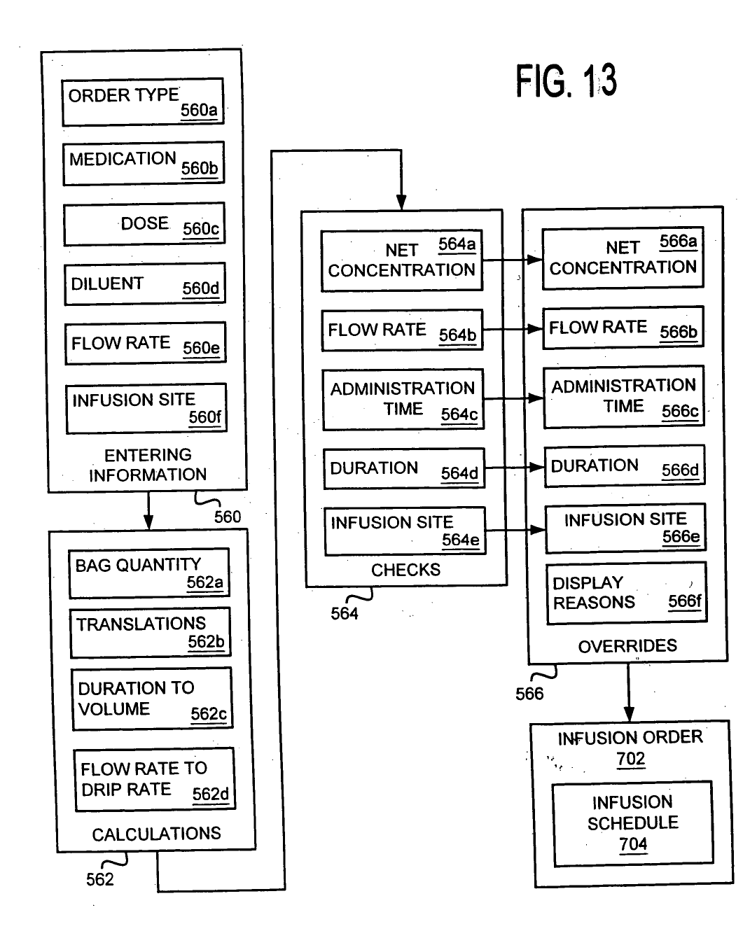

Infusion order creation 504 includes functional blocks used to create infusion orders. Infusion order creation 504 includes functions similar to those described in reference to prescription generation 304 (FIG. 9). Infusion order creation 504 includes, but is not limited to, entering information 560, calculations 562, checks 564, and overrides 568. Infusion order creation is further described below in reference to FIG. 13. The result of infusion order creation is an infusion order 702 (FIG. 13). Infusion order 702 generally includes an infusion schedule 704 (FIG. 13).

Infusion orders can require authorization as described in reference to block 308 (FIG. 9). In FIG. 13, prescription authorization by the pharmacist and prescription authorization by the physician are considered separately in functional blocks for pharmacy authorization 508 and physician authorization 510. Physician authorization 510 may not be required if the infusion order is initiated by the physician. The infusion order generally requires pharmacy authorization 508 and physician authorization 510 if the order is generated by a clinician at the treatment location 106, other than the pharmacist or physician. However, if medication 124 is required immediately, the infusion system 210 allows administering clinicians to bypass prescription authorization 508 and physician authorization 510. In the case of emergency orders or non-emergency orders for routine medications, the infusion system 210 can determine there is no information stored in the patient care system 100 related to the medical treatment the clinician 116 desires to administer to the patient 112. If the infusion system 100 recognizes the clinician 116 as having the authority to initiate the desired medical treatment, the system 210 allows for the administration of the medical treatment without going to blocks 508 and 510. This authorization is then obtained following administration.

Infusion order preparation 506 can be accomplished in a number of locations throughout the medical facility such as, but not limited to, the pharmacy, the nursing center, on the floor, and the treatment location 106. Preparation 506 includes providing instructions for preparing the medication 124 and minimizing the possibility of errors in medication preparation.

Medication administration 512 takes place at the treatment location 106. The infusion system 210 is designed to make the administration of the order as efficient and accurate as possible. The infusion system 210 provides the administrating clinician with the tools to administer the right medication to the right patient in the right dose, with the right pump settings, at the right time, and via the right route. Should an alert, alarm, reminder, or other message be appropriate in assisting the clinician with the administration of the medication, the medication administration module provides a status information output to the messaging module 520. In response to the status information output, the messaging module 520 forwards a related text message, audible indicator enable, or both, to one or more electronic computing devices.

As known by those having skill in the art, infusion orders are frequently modified. Infusion system 210 provides modifications 514 to account for infusion order modifications. Modification 514 includes modifications to infusion duration, flow rate, infusion site, and stop orders 516. Modification 514 also includes the functional blocks required to implement infusion order modifications.

The infusion system 210 can include patient care system 100 wide defined stop orders 516. Changes in patient status may generate messages 520 for appropriate action. The infusion system 210 coordinates with the transfer interface 310 to automatically stop orders 516 upon discharge or death.

The system 100 includes inventory and billing module 518. Inventory and billing 518 allows the financial transactions associated with patient care to proceed with a minimum of human intervention. The completion of medication administration 512 can trigger patient billing through the billing interface 312. The billing interface can include an HL7 interface. If patients are to be charged based on completion of infusion order preparation 506, the inventory and billing system 210 includes a crediting process. The crediting process can be triggered when infusion bags are returned to the pharmacy for disposal or re-entry into the pharmacy inventory management system.

The infusion system 210 includes a messages module 520 for communicating with entities throughout the patient care system 100. In particular, the messages module 520 sends text messages, audible indication enables, or both, to one or more electronic computing devices within the patient care system 100. The messages are sent in response to a status information output provided by the medication administration module or other infusion system modules within the patient care system 100. The messages relate to the status information output and, as such, provide alerts, alarms, reminders, or other messages appropriate in assisting the clinician with medication administration.

For example, when a physician enters a new order, messaging appears in the pharmacy to alert the pharmacists that an infusion order requires authorization. Likewise, when infusion orders are appropriately authorized, the clinician 116 receives messaging on digital assistant 118 to alert the clinician 116 that the infusion order should be administered according to the infusion schedule 704. Overrides 566 may generate messages 520 for the physician and/or the pharmacy. The infusion system 100 can distinguish between system-wide and sub-system overrides in determining whether it is necessary to generate a message 520. Messaging 520 includes messages received and/or sent to the central system, the pharmacy, the physician, billing, and inventory.

The system can present clinicians 116 with personal computer display views. The personal computer display provides a view summarizing outstanding clinical problems for the clinician's patients. The clinician 116 can quickly retrieve detailed information for the patients. The system 100 can also produce an email or page to digital assistant 118, or other communication device, when certain critical patient conditions prevail.

FIG. 11 also depicts some of the communication paths that occur in patient care system 100. The highlighted communication paths are presented for ease in describing the infusion system 210. Those having ordinary skill in the art recognize that when patient care system 100 is practiced on a network the various functional blocks can communicate with each other via the paths highlighted in FIG. 11 and via alternate paths that are not shown in FIG. 1I. Setting system parameters 502 includes communicating data related to the system parameters to infusion order creation 504, via path 522, and/or receiving data from infusion order creation 504 and providing data informing infusion order creation 504 of how the received data relates to the system parameters.

Infusion orders can be passed directly, via path 524, to infusion preparation 506. Infusion orders can also be passed to pharmacy authorization 508, via path 526 and/or to physician authorization, via path 528, before being sent to preparation 506. Path 530 highlights the delivery of the medication 124 from the preparation area to the treatment location 106. Delivery can be accomplished using medication treatment cart 132. Paths 532, 534, 536, and 538 highlight that inventory and billing 518 transactions can be tied to a variety of other functions such as, but not limited to, infusion order creation 504, preparation 506, medication administration 512, and modifications 514. Paths 572, 574, and 576 highlight that a larger number of functions and actors involved in patient care system 100 can generate and receive information via messages 520. Path 582 highlights that system defaults 544 can be created and/or modified by the pharmacist. And, path 580 highlights that information, such as infusion orders, is available to a variety of functional units throughout the system 100.

FIG. 12 is a block diagram showing functional components for the setting of system parameters 502 of FIG. 11. Setting system parameters 502 includes, but is not limited to, setting tolerances 542, setting defaults 544, building databases 546, defining functions 548, and determining system settings 550. Tolerances 542 includes tolerances such as, but not limited to, net medication tolerances 542a, flow rate tolerances 542b, administration time tolerances 542c, administration system duration 542d, medication duration tolerances 542e, and site change tolerances 542f. The infusion system 210 can also include separate tolerances for order entry and modifications from the ordered tolerances. For example, separate tolerances can be identified such as, but not limited to, an administration system duration 542d, an order entry maximum infusion duration override availability setting, and an administration maximum infusion duration override availability setting.

A net medication tolerance 542a is a maximum concentration of a medication that is safe to administer to a patient. The infusion system 210 associates the net medication tolerances with medications. Net medication tolerances 542a can be defined in medication identification files in a medication database. During infusion order creation 504, the infusion system 210 can determine the flow rate 560e, the number of infusion bags required 562a for a specified period of time, the concentration of the primary ingredient in each infusion bag, the time period over which each infusion bag is to be administered, and the total volume of each infusion bag. Flow rates can be manually entered or adjusted by altering the final concentration or the duration of each infusion bag. In an embodiment, the infusion system 210 performs a net concentration check 564a (FIG. 13) to ensure the maximum concentration of the medication is not exceeded. However, if at any time while a clinician 116 is modifying the flow rate by adjusting the final concentration resulting in the final concentration of a solution exceeding the maximum concentration of the medication, the infusion system 210 sends a message 520 to the administering clinician. The administering clinician can be authorized override the net medication tolerance 542a. The infusion system 210 can require the clinician 116 to provide a reason for the override.

Infusion system 210 can include adjustable flow rate tolerances 542b and flow rate adjustment tolerances for administration. Flow rate tolerances 542b are optionally defined for all organizational levels of the patient care system 100. The tolerances 542b can be for the entire patient care system 100, or for sub-systems of the patient care system 100. For example, different flow rate tolerances 542b can apply to sub-systems such as, but not limited to, neonatal, pediatric, psychiatric, specific nursing units, and for specific patients. The flow rate tolerances 542b can be specified relative to the original ordered flow rate or relative to the immediately preceding flow rate. The clinician 116 can also specify a flow rate tolerance specific to a particular order.

The infusion system 210 can include a pre-defined indication of whether the administering clinician 116 is permitted to override the flow rate tolerance 542b without requiring a new order. This indication can apply to the entire patient care system 100, a sub-system, or an individual clinician 116.

The maximum infusion duration 542d can be separately definable for the various portions of the patient care system 100. The maximum infusion duration 542d can also be specific to a particular medication 124. A maximum infusion duration override 568d (FIG. 13) can be provided if it is permissible to override the maximum infusion duration 542d at the time of order entry. An administration maximum infusion duration override can be provided to set whether it is permissible to override the maximum infusion duration 542d at the time of administration and which group of users is allowed to do so. If it is permissible to override during order entry and/or administration, the infusion system 210 can define a subset of the clinicians 116 that have the authority to override the maximum infusion duration 542d.

Defaults 544 include defaults such as, but not limited to, medication diluent defaults 544a, diluent quantity defaults 544b, dose defaults 544c, and units of measure defaults 544d. Units of measurement (UOM) defaults 544d include the ability to specify the units of measurement that are most suitable for different portions of the patient care system 100. For example, medication can be measured in different units by physicians, administering clinicians, pharmacists, financial personnel, and medication screeners. The physician's UOM is generally a measurable value such as “mmol”, “mEq”, “ml”, and/or “mg”, as opposed to “vial” and/or “puff.” The physician's UOM is used for tasks such as ordering and entering information 560.

The Administering clinician's UOM is generally a value that reflects the UOM the medication will be administered in, such as “puff”, “tbsp”, and “tab”. The Administering clinician's UOM is used during medication administration 512. The Administering clinician's UOM can also appear on documentation such as administration reports, admixture fill and manufacturing work orders.

The pharmacy UOM is generally a value that reflects the physical form the medication is dispensed in such as “tab”, “vial”, “inhalator”, and “jar”. The pharmacy UOM is used in preparation 506 and in stocking and dispensing systems. The financial UOM is generally a value used to calculate the financial figures that appear on bills and invoices. The medication screening UOM is generally used when screening the medication.

Units of measurement defaults 544d can be specified using a check-box table where checkmarks are placed in a table correlating the various UOMs with the users of the UOMs. The infusion system 210 can use the same UOM for more one function. For example, the physician's UOM can be the same as the pharmacist's UOM. Setting defaults 544 include data necessary to coordinate the various UOMs. For example, UOM defaults 544d can include the multipliers and dividers necessary to create a one-to-one correspondence between the various UOMs. The UOM defaults 544b can be changed to suit the desires of the individual clinicians. However, the one-to-one correspondence should be maintained by the patient care system 100. The infusion system 210 can be designed to maintain a history of medication unit defaults.

The infusion system 210 can also include a medication measurement suffixes. The medication measurement suffixes can default during order entry. The medication measurement suffixes can be common units of measuring a medication and can include units related to patient characteristics such as body surface area and weight. Medication measurement suffixes can be designated per drug, per order type, per does, and per UOM.

Building database 546 includes building databases and/or portions of a single database such as, but not limited to, preparation area 546a, additive information 546b, solution 546c, premix definitions 546d, favorites 546e, timing override reasons 546f, flow rate override reasons 546g, translation tables 546h, flow rate description 546i, equipment and routing information 546j, and message trigger 546k.

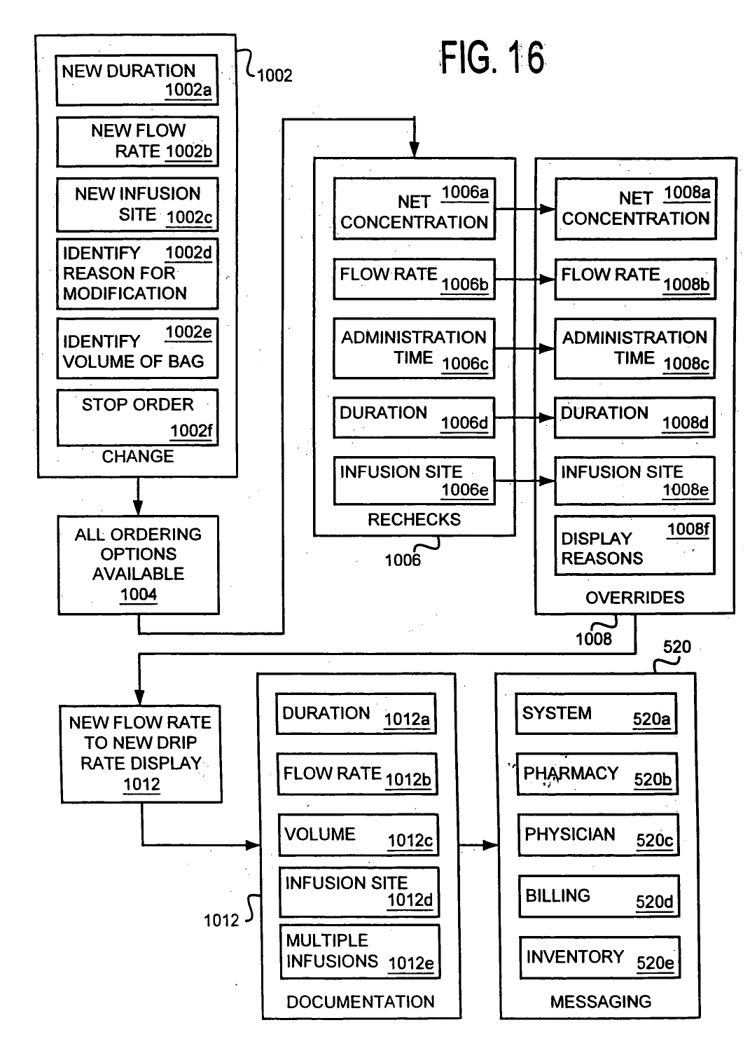

Timing override reasons 546f include displayable reasons for modifying the timing of infusion orders. For example, timing override reasons 546f can include a stylus selectable reason for digital assistant display 118a for administering an infusion order at a time other than the time specified in the original infusion order. If the clinician 116 administers a medication outside the ordered administration time tolerance 542c, the clinician 116 can be required to choose a reason code for the modification from displayed reasons 1008f (FIG. 16). An example of other reason codes includes, but is not limited to, PRN administration reason codes and codes for stopping an infusion.

Medications 124 and/or infusion orders can have flow rate tolerances, including system flow rate tolerances 542b. The infusion system 210 can include flow rate override reasons table 546g. Flow rate override reasons 546g are notations that the clinician 116 can choose from, and/or supply, if the clinician 116 needs to change the flow rate beyond the bounds defined by the flow rate tolerance 542b. The infusion system 210 can include a defined message trigger 546k indicating whether or not a message should be sent to the patient's physician if a clinician 116 overrides an order defined flow rate tolerance. The infusion system 210 can also include defined message triggers 546k indicating whether or not a message should be sent, and to whom, if a clinician 116 overrides a tolerance, such as flow rate tolerances 542b, defined at a level other than the order.

The infusion system 210 can include translation tables 546h such as, but not limited to, a flow rate translation table, a varying ingredient translation table, and varying flow rate translation table. Flow rate translation includes translating an infusion order into a flow rate defined by volume/time where the order is originally specified in any way such as, but not limited to, dosage/time with a particular concentration, volume per unit of weight/time, dosage per unit of body surface area/time, and total dosage and duration.

Varying ingredient translation includes translating a plurality of flow times of infusion orders with varying ingredients in separate infusion bags into the flow rate for the infusion bag currently being administered. Orders with varying ingredients include orders such as, but not limited to, sequencing orders. In sequencing orders, different bags have different ingredients and potentially different flow rates.

Varying flow rate translation includes translation of infusion orders with varying flow rates into the flow rate for the current solution being infused. Varying flow rate orders include orders such as, but not limited to, tapering dose orders and alternating dose orders.

The infusion system 210 can include predefined infusion flow rates 542b. The predefined infusion flow rates 542b can be associated with flow rate descriptions 546i to permit selection from a drop-down list as a shortcut from keying in the flow rate.

Defined functions 548 includes functions such as, but not limited to, preparation area function 548a, bag duration function 548b, verify override requests function 548c, duration to volume function 548d, duration to flow rate function 548e, and flow rate to drip rate function 548f. The infusion system 210 can include a duration-to-volume function 548d to determine the amount to be infused per the infusion order. Flow rate to drip rate function 548f uses information about the medical device 330 to convert flow rates to drip rates.

Determined settings 550 includes settings such as, but not limited to, override authorities 550a, flow rate precision 550b, volume precision 550c, and time precision 550d. The infusion system 210 can, if desired, determine the total volume of infusions and the flow rate(s) of the infusion order. If these numbers are determined, it is desired to round the calculated values to flow rate precisions 550b and volume precisions 550c that are comprehensible to clinicians 116 such as the physician, the pharmacist, and the nurse. Flow rate display precision 550b can be set to display the flow rate to a set number of decimal places. Various parts of the patient care system 100 can independently determine the precision for displayed flow rates. For example, the infusion system 210 can display to one decimal place for an adult treatment location, and to three decimal places for a neonatal treatment location. The flow rate precision 550b reflects the service in which the clinician's patient(s) are located. The flow rate(s) of the infusion order can be rounded to a system defined precision. The precision can be same for all infusion orders or be dependent on the patient's service.

Volume display precision 550c can similarly be set to display infusion volumes to a set number of decimal places. Settable time precision 550d can be used to calculate the administration duration period based on flow rate if the infusion is a single dose infusion or an intermittent infusion. The total volume of each infusion bag calculated is rounded according to the volume precision 550c. The administration time is rounded by the infusion system 210 according to the set time precision 550d. The time precision 550d can be the same for all infusion orders regardless of the patient's service or may be service specific.

FIG. 13 is a block diagram showing functional components for infusion order creation 504 of FIG. 11. Infusion order creation 504 includes functional blocks for creating infusion orders. Infusion order creation 504 includes entering information 560, calculations 562, checks 564, and overrides 568. Entering information 560 can include functions such as, but is not limited to, identifying the order type 560a, identifying the medications 560b, identifying the dose 560c, identifying the diluent 560d, identifying the flow rate 560e, and identifying the infusion site 560f.

Infusion order creation 504 is linked to infusion bag preparation 506, and infusion bag delivery (path 530), medication administration 512, and infusion order modifications 514. Infusion order types 560a include order types such as, but not limited to, single dosing, load dosing, intermittent dosing, and continuous. Continuous infusions include alternating infusions, sequencing infusions, tapering infusions, and titrating infusions. Upon selection of the first medication 560b in an infusion order, an infusion order type 560a form for the medication may default. The ordering clinician can have the option of selecting a different order type. The dose 560c and unit of measure 544d can also default. The unit of measure 544d can be correlated with the medication and/or the dose 544c. The infusion system 210 can include a default diluent, or several default diluents, for the medication. One default can be identified as a preferred diluent. A description can be associated with the diluent to assist the ordering clinician to decide which diluent to select. The diluent description can include a reference avoiding use of a particular diluent if a patient is hypertonic.

The infusion system 210 can also allow additional infusion order subtypes 560a based on the previously mentioned infusion order types. Additional infusion order subtypes 560a can include, but are not limited to, TPN infusion orders, chemotherapy continuous infusion orders, piggyback infusion orders, and large volume parenteral infusion orders. The infusion order subtypes can be accessed from different parts of the infusion system 210 allowing sorting and filtering of infusion orders according to the subtypes. A special label format for each infusion order subtype can also be defined to further customize infusion order subtype orders and associated pharmacy workflow.

When searching for a medication 114 during infusion order creation 504, the medication 114 can be flagged as additive and/or a solution to aid the clinician 116 in creating the infusion order. This designation can be made in a medication identification file.

Medication dose 560c can be determined in a number of ways such as, but not limited to, according to body weight, body surface area, and entered according to rate. When the flow rate is not entered, the infusion system 210 calculates the flow rate according to the dose and time period specified. The ordering clinician can specify the diluent 560d and its quantity. The pharmacy can provide a default for such parameters—see line 582 (FIG. 11). A check 564 can be performed to ensure the net concentration 564a for the medication 560b and the flow rate 564b are appropriate.

The infusion system 210 can identify and/or calculate flow rates 560e based on the patient's weight, body surface area, and/or a specified frequency and duration of therapy. The ordered flow rate 560e is checked 564b against the flow rate tolerances, such as system flow rate tolerance 542b. The net concentration of the medication 124 can be checked 564a against net concentration tolerances, such as the system net concentration tolerance 542a.

In an embodiment, flow rate 560e can also include displaying descriptions of default flow rates to facilitate the entering of orders. Flow rate 560e can reference flow rate descriptions database 546i.

Calculations 562 can include calculating the dose based on patient weight and/or height (possibly provided by ADT interface 310), the drug amount, diluent volume, concentration, or rate.

Calculations 562 can include, but are not limited to, calculating the flow rate, if not specified in the prescription, the bag quantity 562a or number of infusion bags required for a specified period of time, the time period over which each infusion bag is to be administered, and the total volume of each infusion and infusion bag based on the concentration of the ingredients in the solution. Flow rates, volume to be infused, and/or duration can be modified. If modified, the infusion system 210 automatically calculates dependent quantities, based on calculations, if the maximum dosage for the ingredients in the concentration would be exceeded as identified in the ingredient's medication file, the patient care infusion system 210 alerts the pharmacist and/or clinician 116 and can ask for a reason code for the adjustment.

Calculations 562 can include calculations such as, but not limited to, bag quantity calculations 562a, translation calculations 562b, duration to volume calculations 562c, and flow rate to drip rate calculations 562d. Checks 564 include a variety of checks that an infusion order can be subject to. The checks include checks such as, but not limited to, a net concentration check 564a, a flow rate check 564b, an administration time check 564c, a duration check 564c, and an infusion site check 564e. If an infusion order fails a check 564, the clinician 116 may be able to override the check. Overrides 568 can include overrides such as, but not limited to, a net concentration override 566a, a flow rate override 566b, an administration time override 566c, a duration override 566d, and an infusion site override 566e. Overrides 568 can generate messages 520 for the physician and/or the pharmacy. The infusion system 210 can distinguish between system-wide and subsystem overrides in determining whether it is necessary to generate a message 520.

Overrides can include an indication of whether clinicians have the authority to override a tolerance. For example, flow rate override 568b can provide an indication of whether the clinician entering the infusion order has the authority to override the system flow rate tolerance 542b. This indication can apply to the patient care system 100 or a sub-system. Duration override 568d can provide an indication of whether the clinician 116 entering the infusion order has the authority to override the system duration 542d. This indication can apply to the patient care system 100 or a sub-system.

Overrides 566 also include displaying of reasons for the override 568f. Reasons for the overrides 568f can be selected by the clinician 116 from drop-down menus.

The result of the infusion order creation 504 is an infusion order 702. Infusion order 702 can include an infusion schedule 704. The infusion system 210 can look ahead a period of time and generate the infusion schedule 704—so long as the infusion order 702 is active—for infusion bag filling for that time period, or longer if specified on demand. The ordering clinician is not required to specify an end-date for the infusion order. The infusion system 210 can include automatic scheduling of infusion bag delivery based on infusion system 210 defined tolerances 542.

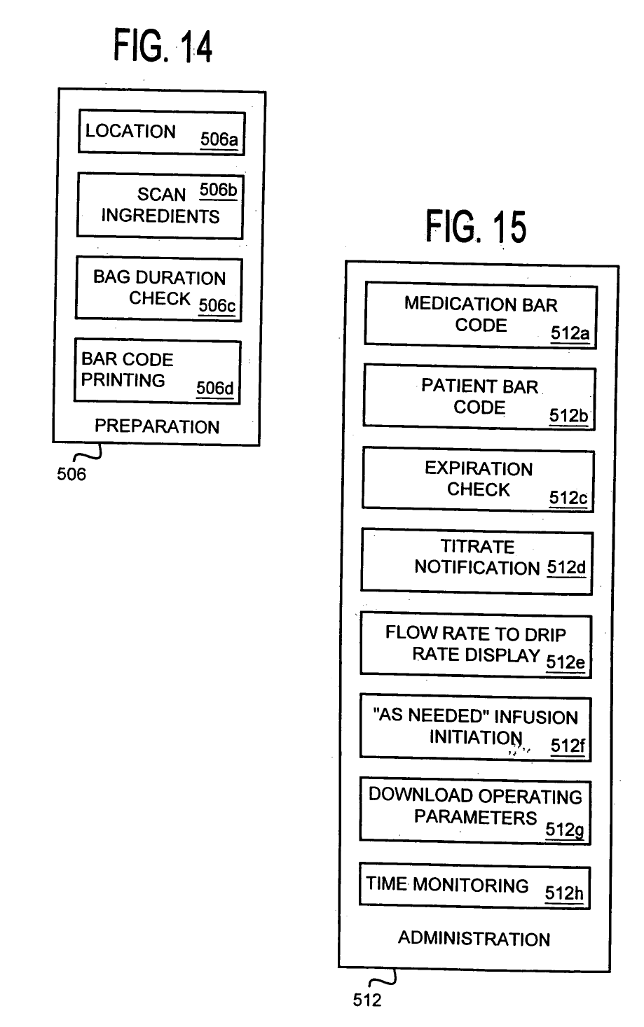

FIG. 14 is a block diagram showing functional components for infusion order preparation 506 of FIG. 11. Infusion preparation 506 includes functional blocks for preparing infusion order 702 (FIG. 13). Infusion preparation 506 can include, but is not limited to, determining preparation location 506a, scanning ingredients 506b, bag duration checking 506c, and bar code printing 506d for medication labels 124a. Bar code printing 506d can include the functions described above in reference to print label 326 (FIG. 9).

After infusion orders are entered into the infusion system 210, preparation instructions are routed to a preparation location. The preparation location depends upon the infusion system's 100 preparation program 506 and the infusion components. The infusion system 210 can include adjustable databases, such as preparation area database 546a that specify where the infusion order is to be prepared. The infusion order can be prepared in the pharmacy or in a remote location, such as on the floor or at the treatment location 106. The clinician 116 is guided through the preparation process, including bar code verification of ingredients, using event management information that can be displayed on digital assistant 118 or another device having a display.

The medication label 124a identifies the ingredients and ingredient concentrations. The medication label 124a can be printed in any location. The medication label 124a preferably includes bar code printing 506d. Bar code printing 506b can include printing a bar code label 124a for each infusion bag. The label 124a assists in ensuring that the correct medication is administered at the correct times and/or in the correct sequence. Alternating and sequencing infusion orders are particularly vulnerable to sequencing and timing errors. Bar code printing 506b can include printing a unique bar code label for every bag in infusion order 702. Bar code printing 506b can also include printing a bar code label 124a that uniquely identifies the combination of ingredients in an infusion bag and the concentration of those ingredients. The bar code for medication 124 can include a prefix, a suffix, and the national drug code (NCD). In an embodiment, the bar code can also include a lot and expiration date. Alternatively, a separate bar code can be provided to include the lot and expiration date.

FIG. 15 is a block diagram showing functional components for medication administration 512 of FIG. 11. Medication administration 512 includes functional blocks that are used to administer the medication to patient 112. Medication administration 512 can include reading a medication bar code 512a, reading a patient bar code 512b, running an expiration check 512c, providing titrate notification 512d, providing a flow rate to drip rate display 512e, providing “as needed” infusion initiation 512f, downloading operating parameters 512g, and time monitoring 512h. The infusion system 210 can also translate orders that may have more than one flow rate, such as tapering and alternating orders, into the flow rate for the infusion bag currently being administered. The infusion system 210 can also translate orders having infusion bags with different ingredients, such as sequencing orders, into the flow rate for the infusion bag currently being administered.

Upon administering the medication 124, the clinician 116 scans the medication label 124a. The infusion system 210 includes scanning the bar coded label 24a when initiating the administration of the infusion order, when changing flow rates, changing bags, and/or stopping the infusion order. Infusion system 210 verifies that the infusion bag having the bar coded label should be administered at that time and is for patient 112. The history of the medication administration, including flow rates and volumes administered, can be captured and maintained.

Some infusion orders require hanging of an infusion bag with the intent of only a partial, specific amount of the infusion bag to be administered. The infusion system 210 allows a clinician 116 to order an amount of an infusion bag to be administered. Most infusion pumps have the ability to define the volume to be administered or the flow rate and time period. Once this time has elapsed, the infusion pump will automatically prevent further administration. Infusion system 210, as a reminder to the administering clinician, provides a message on the medication label 114a that it is to be partially administered and the appropriate volume to be administered.

Flow rate to drip rate display 512e uses data generated by flow rate to drip rate functions 548f to provide the administering clinician with drip rates for the current infusion bag. During medication administration 512, the clinician 116 can check on the flow rate and other operating parameters using the digital assistant 118. Flow rate modifications 1002b (FIG. 16) are communicated in real-time.

The infusion system 210 can include PRN or “as needed” infusion initiation 512f. “As needed” infusion initiation 512 causes the creation of a new active order and the preparation of the PRN medication. This option can include prompting the clinician 116 to select a PRN infusion from a list of anticipatory PRN orders placed for the patient and defaulting the requested infusion bags to one. The clinician 116 can have the authority to modify the requested quantity of infusion bags.

Downloading of operating parameters 512g can include determining whether the patient identifier associated with the medical treatment and/or the patient identifier retrieved from the wristband 112a, is the same as the patient identifier associated with the medical treatment at the central location. The determination often is made by the first computer, for example, the pharmacy computer 104a. If the infusion system 210 determines the various patient identifiers are not the same, the system can generate an alarm message 520. If the infusion system 210 determines the various patient identifiers are the same, the infusion system 210 can download the operating parameters directly to the medical device 332. The infusion system 210 can send the operating parameters to a medical device 332, such as infusion pump 120.

One benefit of the system program 210 is that the operating parameters for the medical device 332 do not have to pass through digital assistant 118, or any other computer in the remote location, prior to the operating parameters being available to program the medical device 332. Bypassing computers at the remote location eliminates a potential source of errors in administering medication 124 to a patient 112. The operating parameters for the medical device 332 can be sent “directly” to the medical device 332 assuming the various verifications are achieved. In this context, “directly” meaning that the operating parameters can be sent to the medical device without passing through the digital assistant 118, or any other computer in the remote location.

In another embodiment, the infusion system 210 can include an additional block (not shown) where the central computer accepts a second medication identifier. The clinician 116 at the remote location can enter the second medication identifier. The second medication identifier can be a revised first medication identifier. For example, the second medication identifier can be part of the prescription or electronic physician order entry that is the source for the first patient ID and the operating parameters. The infusion system 210 can then confirm the first and second medication IDs are equivalent prior to sending the operating parameters to the medical device. The second medication ID can be replaced by a revised first medication ID between the time the prescription is entered and the time the medication 124 arrives at the treatment location 106. The infusion system 210 will then sound an alarm if the second medication identifier is not equivalent to the first medication identifier that was included in the medication label 124a. In a further embodiment, the infusion system 210 can include an additional block (not shown) where the operating parameter is used to program the medical device 332.

Various blocks of the infusion system 210, such as block 512, can include displaying treatment information on the digital assistant 118. This can include displaying information that mirrors the information on display 120c of infusion pump 120. The information on display 120c of infusion pump 120 can be supplemented with information about the patient 112, the patient location, and the infusion order. This information can include information regarding multiple channels of infusion pump 120. The displayed information can include information such as, but not limited to, personality, prompt line, status line, operating icons and pump head display. Operating icons include falling drop, stop sign, flow check piggyback, Guardian, and delay start. The pump head display includes information such as the drug label and the infusion rate. Those having ordinary skill in the art are familiar with the displayed information and operating icons described above.

The infusion system 210 time monitoring 512h calculates the time remaining for an order to be completed and the volume of an infusion order that remains to be administered. When the clinician 116 uses the infusion system 210 to administer the infusion order, to make flow rate changes, and to check on the status of an infusion, the infusion system 210 calculates time and volume remaining to be administered and indicates if the calculation indicates a partial bag will be used. For example, on the last bag of an order that is to be stopped before the full volume is administered, and/or on a bag within an order that must be changed before the full volume is administered, the clinician 116 is alerted on digital assistant 118 and/or cart 132. The alert can include a message such as “Please only administer 150 ml.”

Time monitoring 512h includes tracking any modifications made to the flow rate using bar code scanning. The pharmacy is alerted in real time to adjust the preparation 506 of the next required infusion bag according to the modification. Monitoring of preparation 506 and medication administration 512 allows for a just-in-time delivery of medication 124. Just-in-time delivery reduces wastage attributed to discontinued or changed infusion orders. Monitoring also ensures patient 112 safety.

For titrate PRN orders, the clinician 116 is automatically notified of required flow rate changes if the titration conditions in the order indicate that the flow rate must be changed. The infusion system 210 includes defined functions for calculating a conversion of flow rates to drip rates 548f. The infusion system 210 defined values can be adjustable. The infusion system 210 can include automatic translation of flow rate to drip rate 548f to assist the clinician 116 during administration of the treatment.

FIG. 16 is a block diagram showing functional components for infusion order documentation 1012, and the infusion order modifications 514 and messaging 520 of FIG. 11. Modifications 514 include functional blocks used to modify existing infusion orders. Modification 514 can also be viewed as creating new orders to replace existing infusion orders. Modification 514 can include modification changes 1002, generally all ordering options for new orders 1004 are available, rechecks 1006, recheck overrides 1008, and new flow rate to new drip rate display 1010. Infusion order modifications often lead to documentation 1012 and messaging 520. Modifications 514 include the functions described in reference to prescription modification module 336 (FIG. 9). However, modifications 514 are also accessible from other portions of the patient care system 100 such as, but not limited to, prescription entry 324, prescription activation 306, and prescription authorization 308.

Modifications 514 include modifying the duration 1002a, modifying the flow rate 1002b, using a new infusion site 1002c, identifying reasons for modifications 1002d, identifying the volume of an infusion bag 1002e, and processing stop orders 1002f. Clinicians 116 can also change an infusion rate without an order if the patient 112 is complaining of discomfort or to facilitate fluid balance, such as when the patient 112 is vomiting.

Modification changes 1002 include identifying a new duration 1002a, identifying a new flow rate 1002b, identifying a new infusion site 1002c, identifying a reason for a modification 1002d, identifying the volume remaining in the infusion bag 1002e, and stop orders 516. The ordering options available during initial infusion order creation 504 are generally available for modifying the infusion order. Ordering options available during initial infusion order creation 504 include those shown in FIG. 13. Rechecks 1006 and recheck overrides 1008 are analogous to checks 564 and overrides 568 that are described in reference to FIG. 13. New flow rate to new flow rate display 1010 assists the clinician and minimizes the possibility of errors during medication administration 512. The modified infusion order can lead to a modified infusion schedule.

Flow rates are frequently modified at the treatment location 106 for reasons such as to catch-up without changing the schedule for preparation when the infusion has been inadvertently stopped for a short time period. Such modifications may not require new infusion schedule 704 to be communicated to the pharmacy. In other cases, the new schedule 704 should be communicated to the pharmacy or other preparation staff. Flow rate modifications 1002b triggers infusion order scheduling changes and/or messages 520 for appropriate clinicians 116.

When a clinician 116 enters a flow rate modification 1002b into the infusion system 210 at treatment location 106, the clinician 106 can also elect to have the infusion schedule 704 recalculated and sent to the pharmacy. The clinician 116 has the option of requesting new medication labels 124a to be printed by bar code printing 506d module. The new medication labels 124a include data reflecting the new information for any of the previously prepared infusion bags.

The infusion system 210 and/or the clinician can request a modification to the infusion site 1002c. The site can be selected from a list of anatomical representations on a computer screen.

The clinician 116 can be required to identify a reason for the modification 1002d. Reasons stored in databases such as, but not limited to, override reasons for timing 546f and override reasons for flow rate 546g, can be displayed for easy identification by the clinician 116. There can be a separate hard-coded reason for physician ordered modifications. For physician ordered modifications, the clinician 116 can be requested to identify the physician.

Prior to implementing the modification, the volume remaining in the current infusion bag is identified 1002e. The clinician 116 can be offered the option of accepting a volume calculated from a displayed value of pre-modification flow rate and/or volume.

If desired, the current infusion can be stopped 1002f. If stopping the order is not required, for example the same infusion bag can be used with a new flow rate and/or a new medication added, the old flow rate can be identified and compared to the modified flow rate.

Any infusion bags that were previously prepared can be checked for expiration based on the new infusion schedule 704. When an infusion order is resumed following either a temporary stop or a hold order, the expiration check can be done regarding expiration of solutions that have already been prepared.

The new infusion schedule 704 is used to control the preparation 506 in the pharmacy or other preparation site. A system default 544 can be set for whether or not any prepared bags should be credited to the patient 112, through the billing interface 312, and whether or not they should be credited to inventory.

Infusion order changes 1002 include all ordering options available 1004 for new orders. The modified flow rate can be rechecked 1006 for rules and tolerances such as, but not limited to, net concentration 1006a, flow rate 1006b, administration time 1006c, duration 1006e, and infusion site 1006f. Overrides 1008 can be available for modifications that are outside of tolerances. The infusion system 210 can display reasons 1008f for overrides and for administering medications at times other than that specified in the original order. The clinician 116 can be required to identify a reason for the modification.

The infusion system 210 can offer the clinician 116 a display indicating the modified drip rate associated with the modified flow rate 1012. The displayed information can be calculated by the flow rate to drip rate 548f defined function. The infusion system 210 can also be provided with descriptions of typical infusion tubing used within the infusion system 210 for use in calculating drip rates.

A modification results in the infusion system 210 validating the expiration of the infusion bag and providing a message to the clinician 116 if the infusion bag expires prior to the completion of the order. The message can request that the clinician 116 contact the pharmacy. The validation of the expiration of the infusion bag for solutions such as, but not limited to, premixed solutions and solutions manufactured outside of the infusion system 210, may include parsing the scan code.

Flow rate override 1008b can provide an indication of whether the clinician 116 modifying the infusion order has the authority to override the ordered override without requiring approval for a new infusion order. This indication can apply to the patient care system 100 or a sub-system.

Documentation 1012 captures infusion order information in real-time. Documentation includes documenting multiple infusions being administered at the same time and infusion modifications such as, but not limited to, duration changes 1002a, flow rate changes 1002b, volume changes 1012c, and infusion site changes 1002d.

The infusion system 210 can assist the clinician 116 in capturing all changes in flow rate as the changes are occurring. The clinician 116 can change the flow rate as called for in the order, such as to decrease a morphine infusion flow rate from 4 ml to 2 ml. Though the infusion system 210 may recognize the change as a new order, the infusion system 210 may be configured to avoid duplication so that the modified order does not result in the generation of a new bag.

Documentation 1012 includes the ability to document changes such as, but not limited to, an infusion that is stopped temporarily, discontinued, and/or restarted. The clinician 116 may stop infusion for a variety of reasons, such as the infusion site having been compromised, the infusion has been dislodged, and/or the infusion may be heparin/saline locked to facilitate the movement of patient 112. The infusion can be resumed when a new site/infusion has been reestablished. However the length of time this may take is variable and is generally recorded by the infusion system 210.

Government regulations often require tracking of every step in the process of infusion administration. Infusion system 210 allows the administering clinician 116 to document flow rate modifications on a digital assistant 118, or other computer device, by scanning the medication label 124a and adjusting the flow rate 1002a based on a tolerance, such as a tolerance created by set tolerance 542. A flow rate modification 1002b corresponds in real time with the associated pharmacy's infusion schedule 704 to ensure just-in-time inventory management of infusion bags to the patient treatment area 106. Documentation 1012 may allow order backdating under some circumstances.

The infusion system 210 includes the ability to document the infusion site 1012d and multiple infusions 1012e for multiple infusion sites. In many situations a patient 112 can have multiple medications 124 and “y-ed” infusions so that the some infusions are running into one site and other infusions are infusing into another site. For example, morphine infusion, antibiotics and normal saline infused into the right arm (site 1) and TPN and 2/3 & 1/3 running into a double lumen CVL (site 2). The infusion system 210 allows clinician 116 to document which site the various fluids are infusing through. In treatment locations 106, such as intensive care units, many more than two infusions may be running into one line or one lumen. Clinicians 116 are able to indicate which lumen of a CVL the infusion or medication is running into.

The infusion system 210 includes the ability to document the site location 1012d for infusions and any site location changes. Infusion sites are frequently changed due to occlusions or policy. Therefore, clinicians 116 must document a change in the site location if an infusion becomes dislodged and was subsequently restarted.

The infusion system provides for centralized device configuration. Operating parameters for medical devices 332, such as infusion pump 120, often include defaults and/or tolerances. The defaults and/or tolerances can reside in the infusion system 210, for example flow rate tolerance 542b, and/or in a memory associated with the device 332. For example, infusion pumps 120 can include a database having a table of medications having associated flow rate tolerances. If the clinician 116 enters a flow rate that is beyond the associated flow rate tolerance, the clinician 116 is warned and then can be allowed to proceed—or prohibited from proceeding. Devices 332 such as heart rate monitors can also have configurable tolerances for alerts. In addition to alerts, many other characteristics can typically be configured for devices 332 such as: network name, IP address, polling frequency, and colors. The infusion system 210 includes configuring medical devices 332 individually or in groups from one or more central computers.

System configuration parameters can be defined for a first type of medical device. The system configuration parameters are sent and accepted by the first type of device unless the particular first type of device has more specific configuration parameters that apply to that particular first type of device. For example, a first plurality of a first type medical device can be located at general care treatment locations. A second plurality of the first type of medical device can be located at an intensive care treatment location. The general care treatment location may not have specific configuration parameters while the intensive care treatment location does have specific treatment parameters. System configuration parameters will apply to all of the first type of medical devices throughout the infusion system 210, i.e. the devices in the general care treatment locations, unless specific configuration parameters apply, e.g. the intensive care treatment location.

For each type of device, specific configuration parameters that apply to all devices of that type across a particular grouping of the devices override the system configuration parameters if a particular device belongs to the group having such a definition, unless the specific configuration parameters are overridden at an even more specific level within the infusion system 210. The groups might be defined as a clinical service, a nursing unit, and/or a combination of service and nursing unit.

For each type of device, the user can define sets of configuration parameters that apply to all devices of that type being used for operations with specified ranges of attributes that override any other definition. In a hospital the operations might consist of infusion orders and the attributes might include patient weight, drug, patient disease state, and patient acuity.

Devices can be identified as part of a general group, a specific group, and/or to be associated with a particular patient by including the device address in a table in a database. General or specific configuration parameters can then be sent to the device according to the identification of the device. The specific configuration parameters can then be read back to the infusion system 210 and compared to the originally sent configuration parameters to verify the original configuration parameters were correctly received by the device 332. If the configuration parameters were not correctly received, the infusion system 210 can provide a message 520 identifying the discrepancies or the communication failure.

The infusion system 210 can detect changes to configuration parameters made at the device, rather than through a central computer, and send a message and/or alert 520. The infusion system 210 can also poll the devices to verify their configuration parameters. If system and/or specific configuration parameters change, the changes can be propagated to all devices 332 identified in the system as belonging to the group according to the groupings identified in the infusion system 210.

Throughout this document and the related claims, “central location” and “remote location” are relative terms to each other. A “remote location” is any location where a patient is receiving treatment through a controlled medical device, such as a patient treatment location 106 where patient 112 is receiving treatment through an infusion pump 120. “Central location” is any location, other than the remote location, where parameters for operating the medical device are accessible such as, but not limited to, the location of the pharmacy computer 104 and the central system 108. In a typical arrangement, several remote locations, such as treatment location 106, are in communication with a central location.

In an embodiment, the system can automatically provide clinicians with information associated with one or more medications via pop-up windows. Preferably, a medication table is entered into the central database 108b. The medication table can include the generic name of one or more medications, and any trade names associated therewith. Linked to each medication within the medication table are respective messages for display via pop-up windows. The messages can be defined by the health care facility, or predefined by the system provider.

Preferably, the messages associated with each medication pertain to: 1) hazards associated with the medication, such as in handling or exposure thereto; 2) how the medication is to be administered by a clinician; and 3) physician reference information about medication.

The pop-up windows are displayed when a medication is selected or entered into a computing device such as: when the medication is being ordered by a physician via the CPOE; when the medication is being processed by the pharmacy or the like; and when the medication is being administered to a patient by a clinician. In an embodiment, when the selection or entry of a medication has been made on a computing device at a remote location, the database within the central system 108 is accessed wherein at least one of the pop-up window messages associated with the medication is provided to the remote computing device for display to the clinician.

Preferably, at least one of the pop-up window messages associated with a medication is provided for display upon the initiation of a specific step in the medication order, process, and administration procedure. For instance, upon entry of a medication order into a computing device such as the CPOE, a pop-up window is displayed with a message regarding physician reference information about the medication and, in an embodiment, another pop-up window can be displayed regarding hazards associated with the medication. Then, upon processing of the order by a pharmacy or the like, one or more pop-up windows are displayed on a computing device within the pharmacy 104 for providing general information about the medication, and possible hazards associated therewith. Next, when the order is being administered by a clinician, one or more pop-up windows are displayed on a computing device associated with the clinician (i.e., handheld 118) for providing information about administration of the medication, and, in an embodiment, possible hazards associated with the medication such as how the medication is to be handled.

Preferably, the pop-up windows displayed on a computing device are specific to the step in the medication order, process, and administration procedure that is being carried out by a clinician. For instance, the pop-up window containing physician reference information is preferably not displayed to the nurse, via handheld device 108. Nevertheless, in an embodiment, the user or hospital can define when, and if, a pop-up window should be displayed when a medication is selected or entered into a specific computing device.

It is also preferred that the pharmacy define when, and if, a pop-up window is to be displayed. For instance, pop-up windows are preferably not displayed for common medications. Instead, pop-up windows are preferably displayed for medications wherein the pharmacy or healthcare facility believes that the additional information within the pop-up window will assist in the ordering, preparing, or administration of the medication.

A method of administering a medication with the infusion system 210 is described below. The method includes the ability to modify the infusion order. The modifications include modifications to the flow rate, the infusion site, temporary stops to the infusion, restarting the infusion, and hanging a new medication 124 container. The method includes: scanning a bar code associated with the patient 512b; scanning a bar code associated with the medication 512a; if the infusion is an admixture, validating the expiration 512c; selecting a reason for the modification 1002d; and recording the remaining volume of the infusion bag or accepting the value calculated from the previous volume and flow rate 1002e. The validation of the expiration 512c of the infusion bag can include the use of an admixture table and/or a bar code.

The reason for the modification may come from a defined table 546g. The reason for the modification may also include a hard-coded value for physician-ordered changes. When the hard-coded value is selected, the clinician 116 is prompted to select the physician from a list of physicians. The attending physician can be the default in the list of physicians.

There may be a quick select feature to halt the administration of the medication 124, for example stop order 12002f. If the quick select is not chosen, the following steps can be included: recording the flow rate and/or accepting the previous value for the flow rate—the previous value is displayed on the digital assistant display 1118a, the infusion pump display 120c, and/or the medical cart 132; comparing the previous flow rate to the ordered flow rate—this comparison can be accomplished by using infusion system 210 or subsystem rules and tolerances; displaying appropriate messages; conversions between flow rates and drip rates can be displayed 1012—the conversions can be calculated based on infusion system 210 defined drip-rate conversion tables 548f. The infusion system 210 typically uses descriptions based on the tubing used to make it easy for the clinician 116 to select the correct drip rate conversion.

Changing the flow rate triggers the infusion system 210 to validate the expiration of the infusion bag(s) based on scheduled flow rate. If the solution expires before or during the administration, send a message to the clinician 116, such as “This solution will expire during the scheduled administration period. Please contact the pharmacy.” If it is a premixed infusion bag and/or a customized infusion bag, validate the expiration by parsing the scan code, if possible. Accept the previous infusion site or select a new infusion site location from a list or a graphical anatomical representation. Then recalculate the schedule 704 to implement pharmacy restocking.

Infusion system 210 can include biometrics for identifying patients and clinicians 116. Prior to allowing a clinician 116 to access the infusion system 210, the infusion system 210 accesses information related to the identity of the clinician 116. The infusion system 210 can identify the clinician by using a device, such as a bar code reader, to read the clinicians' badge 116a. The system can also use biometrics to positively identify the clinician 116, to assure the clinician is an authorized user of the system, and to determine whether the clinician 1176 has authority to access portions of the infusion system 210. The infusion system 210 can require a combination of the clinician badge 116a, or other key, and a verified biometric match in order to grant the clinician access to the infusion system 210. The system can also be configured to terminate access to the infusion system 210 when the clinician badge 116a is removed from the vicinity of the device used to read the clinician badge 116a, or other key.

Biometrics is the technology and science of statistically analyzing measured biological data. One field of biometrics is that of determining unique physical characteristics, such as fingerprints. Biometrics makes it possible to identify individuals to digital systems, such as infusion system 210. A digital persona is created that makes transactions and interactions more convenient and secure. Biometric features for identification include features such as, but not limited to, fingerprint, face, iris and retina scanning, and voice identification. Biometric devices include a scanning or reading device, software to convert the scanned information into a digital format, and a memory to store the biometric information for comparison with a stored record. Software identifies specific matched points of data that have been processed with an algorithm and compares the data. Unlike passwords, PIN codes, and smartcards, the infusion system 210 biometrics cannot be lost, forgotten, or stolen.

The biometric scanner can be associated with the device for reading the clinician's badge 116a. For example, the biometric scanner can be a thumb print reader on the handle of a bar code reader. In other embodiments, the biometric scanner and an electronic key reader can be located on the portable medicine cart and/or the medical device. When the clinician 116 places the electronic key within a specified distance of the medical device, a processor will know the specific individual electronic biometric identification file it should expect. The infusion system 210 preferably prompts the clinician 116 to scan his biometric information. The biometric information is entered into the infusion system 210 with some type of biometric reading or scanning device. A one-to-one comparison is made between the scanned biometric information and the previously stored specific individual electronic biometric identification file. This one-to-one identity comparison is more efficient than comparing one-to-many identity files because it does not require searching an entire clinician database for a match. Instead, only one specific comparison is made. If there is a match, then the clinician 116 is granted access to the medical device 332. If there is no match, the clinician 116 is denied access.

In another embodiment, after the infusion system 210 grants access to the clinician 116, the infusion system 210 terminates that access when the electronic key is removed from the biometric scanner, or the vicinity of the biometric scanner. The vicinity within which the electronic key must be kept can be predetermined and/or may be a variable and programmable infusion system 210 parameter.

In one embodiment, the infusion system 210 includes an encrypted digital fingerprint template, a clinician's name, a login name, and a password. One technology for implementing the clinician identifier includes “IBUTTON 400” technology from Dallas Semiconductor technology. The infusion system 210 can be activated when the clinician places a finger on a fingerprint scanner. If the infusion system 210 finds a match, the infusion system 210 can request the clinician 116 login to the infusion system 210. If the infusion system 210 does not find a biometric match, the system does not allow the clinician 116 to access the infusion system 210.

In another embodiment, the database storing biometric information can be kept in the central system 108, the pharmacy computer 104, and/or the treatment location 106. At the treatment location 106, the database can be maintained in the portable cart, the digital assistant 118, and/or the medical device 332. Such distributed databases allow access to remote devices even if the network 102 is unable to communicate between the various locations. When network 102 communication is reestablished, the remote and central databases can be synchronized with any information modified at the other location so that both infusion system 210 databases are properly updated.

The infusion system 210 provides a closed loop infusion therapy management system. The closed loop begins with a clinician 116 order. Among other methods, the clinician 116 can enter the order through digital assistant 118 and/or medical treatment cart 132. The order is then available in real-time for pharmacy authorization 508 and physician authorization 510. The order is available in real-time as an electronic medication administration record (eMAR). The eMAR is available to the clinician 116 for infusion administration. The infusion system 210 automatically documents medication administration 512 and modifications 514 such as flow rate changes 1002b. Through the process of medication administration 512, the infusion system 210 simultaneously adjusts infusion system 210 and/or sub-system inventory and billing 518. The infusion system 210 also provides event management and decision support data. The infusion system 210 is device independent, meaning that it can be run on workstations, wireless tablets, and handheld digital assistants 100. The infusion system 210 generally runs in real time, however, batch processing and or messaging can be used to coordinate various stages of the infusion system 210 processes.

The closed loop infusion therapy management system includes infusion order entry 560, order preparation 506, and the availability of the status of the infusion. Infusion order entry 560 can be through a number of means such as, but not limited to, the prescription entry module 324, the prescription modification module 336, and the pharmacy interface 316. Computer screen 400 can be employed in entering the infusion order. The status of the infusion provides patient 112 specific usage of infusions and alerts the pharmacy of the need for additional infusion bags.

### Clinical Documentation

A particularly useful aspect of the patient care system 100 is the integration of structural clinical documentation and its associated data with point of care treatment of a patient. Historically, patient clinical documentation was typically performed on paper, and, as such, data captured within the documentation could not easily be re-formatted or extracted for reporting purposes, either in the aggregate or for specific patients. When electronic documentation began, systems were able to provide healthcare organizations, such as hospitals, with pre-defined hard-coded data elements and eventually data elements defined by the healthcare organization. While this data was gathered and stored electronically, it was not always available at key points in the treatment process. The clinical documentation aspect of the present invention expands the usefulness of such data by providing point of care access and interface, as well as more end-user customization, control and flexibility to fully utilize this data, especially through graphical user interface and data customization. Thus, the patient care system 100 includes a system and method providing meta-data clinical for formatting and defining clinical documentation, and enabling high-end user graphical interface flexibility—the end user having the ability to format the display of the assessment.

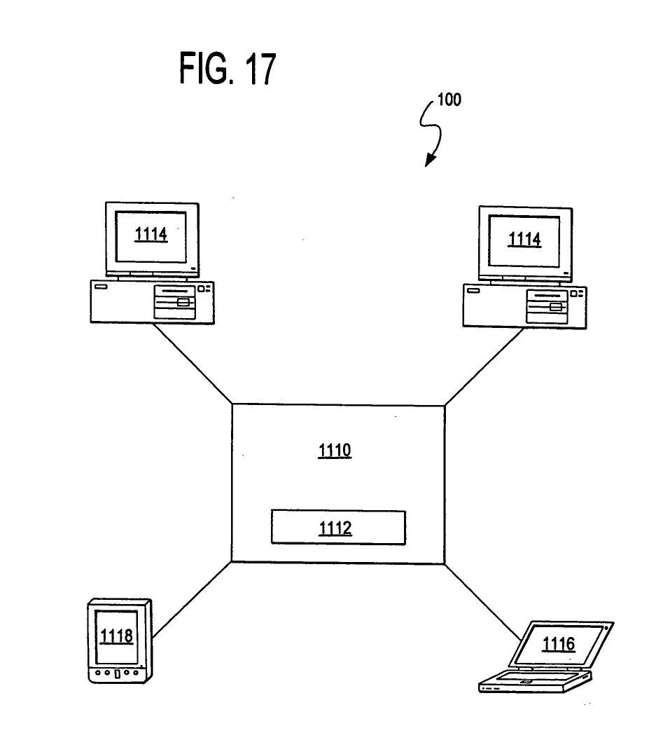

FIG. 17 is a schematic diagram that generally depicts the patient care system 100 having as part of the system an application 1110 including a utility 1112. As already set forth herein, various devices are operably connected to the system 100, such devices including, but not limited to: one or more workstations 1114, a tablet or laptop computer 1116, a hand-held device 1118 (e.g., PDA, phone, pager, radio), etc. Each of these devices are operably connected to the application 1110 within the system. The system, or portions thereof, may utilize wireless technology for connectivity. The application 1110, in conjunction with the system, provides a user the ability to structure clinical documents to be utilized in patient care treatment. Preferably, the system includes a database (not shown) being capable of maintaining clinical information of a patient. The clinical information is capable of being viewed by a patient care provider, e.g., physician, nurses, clinicians, etc., in a structured format defined by the institution. The user can structure the display of information dependent upon the healthcare personnel viewing the information. Similarly, access throughout the system can be dependent upon the role of the healthcare personnel. The devices are capable of communicating information, such as, for example, status of treatment, throughout the system to ensure patient clinical documentation and to provide workflow management and decision support at the point of patient care.

Particular features that facilitate the integration of structural clinical documentation with point of care treatment of a patient shall now be discussed with the understanding that one or more of these features may be incorporated into a particular embodiment of the present invention.

According to a particular aspect of the present invention, an embodiment may include a mechanism having the ability to format a display of an assessment. A clinical documentation module having an end user forms utility enables a healthcare organization to customize its patient documentation. The utility allows for the healthcare organization to define “n” data elements—according to pre-defined data types—that can then be used in groups and sets. When the groups and sets are defined, the utility provides a mechanism to sort the grouped data elements into a logical sequence and allow for the preview of the final created form.

According to another aspect of the present invention, an embodiment may include a mechanism for defining value limits at which point of care decision support messaging is triggered. The end user forms utility includes a healthcare organization definable rules engine that is driven by clinical documentation value entry. According to the data element created in the utility, a healthcare organization can define a user-alert dependent on a clinician's patient documentation. The alert can be utilized as a decision support tool for the clinician at the point of care.

In a particular embodiment, the system is also capable of including normal/abnormal flags. The flags are defined based on value limits. In an embodiment of another aspect of the present invention, the system allows definition of the ranges using only one side of the range such that all inclusive numbers are automatically included rather than the user trying to ensure that all in between values have been covered. After being defined, the system ensures that all rows are sorted appropriately. For each range defined, the value of the normal/abnormal flag and a message column appear. The message column is a free-text field—allowing the user to enter the preferred decision support or message to be presented to the clinician related to values in that range. Entering a message for a given row is optional. In determining which row to apply, i.e., which message to display, when a value is entered, check the lowest criteria first if it matches, check the next criteria if it matches, check the next, if this one does not match, then display the normal/abnormal flag and/or message for the last matched criteria. The system allows for multiple rows to be created for each abnormal flag with each having their own messages. The decision support messaging during entry of documentation is available on a workstation, a tablet, and/or a PDA. As well, the environment can be wireless.

According to another aspect of the present invention, an embodiment may allow for defining normal/abnormal flags to value limits. The system allows definition of ranges—using only one side of the range—such that all values up to the limit, that are not included in a range with a lower limit, are automatically included rather than the user trying to ensure that there are ranges to cover all values. After a range has been defined, the present invention ensures that all rows are sorted appropriately. Next to the range defined, an Abnormal Flag column exists in which the healthcare organization can define a health level seven (HL7) abnormal flag associated with the range. Healthcare organizations can also define a preferred color for each HL7 abnormal flag.

When viewing patient clinical documentation, the actual entered data appears in the corresponding color of the identified HL7 abnormal flag. Furthermore, HL7 abnormal flag text also appears. Both of these visual indicators allow the patient care providers to quickly ascertain patient status. Utilizing a single tool, the system also provides an optional mechanism for clinicians to sort and filter patient results so that only abnormal results are displayed, results are displayed grouped by the value of the abnormal flag, and the like.

Although some health information systems provide visual cues concerning abnormal results, the present invention provides ultimate flexibility in data content, range definition, and corresponding abnormal flag definition.

According to another aspect of the present invention, an embodiment may include a mechanism to define which assessments should appear prior to order entry and whether a reason code is required to proceed without documentation. The present invention allows for a healthcare organization to provide user-defined forms prior to an order being entered into a computer physician order entry (CPOE) system. This allows the healthcare organization to document patient documentation specific to an order directly at the time of order entry. Furthermore, the forms defined by the healthcare organization can integrate existing patient documentation for viewing by a clinician at the time of order entry. Patient documentation attached to an order can be recorded and/or tracked with the order as part of clinical documentation or part of a multi-disciplinary view results window. Also included as part of this functionality is validation defined by the healthcare organization regarding clinician entry of patient documentation at the time of order entry. The healthcare organization is also provided the options of requiring documentation prior to ordering and whether a reason code is required for uncompleted documentation.

According to another aspect of the present invention, an embodiment may include a mechanism to remind a user to document other defined notes when documenting a given note. For example, during configuration of a given assessment or form, a forms utility enables the healthcare organization to advocate to end users that other patient assessments should be documented when completing a particular document. The user may designate an assessment note or groups of notes to be documented sequentially as a result of documenting the initial note. The designation is outlined in an “Assessment Reminder(s)” area of a particular note or assessment. While utilizing a documentation note or groups of notes, the clinician is alerted to the existence of any Assessment Reminder(s) and a list is displayed. The clinician can select which one(s) will be completed subsequently to saving the present assessment. The clinician is guided through each chosen Assessment. Any subsequent assessments also having “Assessment Reminder(s)” are turned off so Assessment Reminders for subsequent documentation are not displayed.

According to another aspect of the present invention, an embodiment may include a mechanism to create reports comprising any combination of structured clinical documentation elements. A clinical documentation module of the application includes an end user forms utility that allows a user, e.g., hospital, to customize patient documentation. Utilizing a meta-data system within a relational database enables the healthcare organization to query a patient database for generating patient-specific reports or aggregate cross-patient reports.

According to another aspect of the present invention, an embodiment may include a mechanism for searching having a variable search value, e.g., search for last N values. Within a multi-disciplinary patient View Results window of the structured-clinical-document system, the healthcare organization and end user are provided with the ability to define a period of time the system will utilize to search entries recorded for assessment documentation or other patient results and to display these results. Alternatively, the search can utilize an amount of entries to be searched, regardless of the time period. This feature, “Look Back,” is discussed later. The system simultaneously displays the last n results that were entered for the note or groups of notes for the patient in question when the user enters a new result. Also, when lab results are viewed the clinician can choose to view the last “n Results.” This functionality is also available on other screens, e.g., panels of lab results within the system.

According to another aspect of the present invention, an embodiment may include a mechanism that enables a view of structured documentation in combination with free-text clinical documentation or independently. The clinical documentation module of the present invention provides clinicians with the ability to view assessments and other clinical notes in both a free-text format and a more structured, pre-defined format. It is often desirable to view a patient's total results, both structured documentation and free-text documentation notes, together on one screen. Such functionality is provided by the present invention wherein the user is given the ability to determine which types of structured documentation results are to be viewed in the clinical documentation window. In the application, the result-oriented category (subcategory) names appear on an active window along with the regular free-text documentation categories. The clinician can select a result-oriented category whereby the results recorded for that patient and pertinent to that category are displayed. The information is not stored in free-text clinical documentation, but may be retrieved for viewing.

According to another aspect of the present invention, an embodiment may include a mechanism to define a summary end-user view and a detailed end-user view. During creation of a structured clinical document, the user can define which part(s) of the result should be displayed, which part(s) of the result should be displayed first to the user, and which components to display on demand. Typically, when viewing structured clinical documentation notes and/or results, the user is shown all data entered for a given structured note at the same time. Providing the ability for the user to define which data is displayed enables the user to see a trend for the patient's results more quickly because the additional information is often not necessary when viewing the trend.

According to another aspect of the present invention, an embodiment may include a mechanism capable of creating structured-clinical-documentation forms wherein the user, e.g., healthcare organization, hospital, etc., can specify that a field or multiple fields are mandatory (“Required”). During information entry, if the clinician attempts to save the entry form without populating a required section, an alert is displayed notifying that the specified field is mandatory. Alternatively, a reason explaining why the entry was not made can be recorded. The reason can be provided by the clinician or selected from a pre-determined list The non-entry and its associated reason code can be tracked in the healthcare organization database and reportable at any time.

According to another aspect of the present invention, an embodiment may include a mechanism that allows a user to define conditions for notifying that entered clinical documentation is significantly different from the last entered value; and subsequently, notification of a condition being met. In an effort to notify clinicians of significant results associated with a patient, the system transmits an alarm. Initially, a percentage difference is specified for each data element wherein exceeding the difference initiates the alarm (“Significant Difference Alert”). Preferably, this configuration rule is set when defining data elements/set/groups through the forms utility. When a clinician is recording patient clinical documentation, the significant difference alert message is displayed to the clinician after the value is entered and the condition is met. It is not necessary for the value or result to be saved for this message to appear. Furthermore, if this value is saved, an automated message can be sent in real-time to any other healthcare provider interacting with the patient.

According to another aspect of the present invention, an embodiment may include a mechanism for a user to define conditional parameters for clinical documentation. Utilizing a forms utility to specify a data element, the user can define further data elements that require entry based on the evaluation of the user-entered value for the preliminary data element. When defining the conditional assessment within the utility, the user may also define whether documentation on the data element and/or its conditional documentation is/are required or mandatory. If mandatory, the user may further define if a reason code is required by the user for non-entry.

During the recording of patient clinical documentation, the assessment is dynamically created based on entry of particular patient data. This facilitates the clinician's ability to complete clinical documentation in a timely manner. Additionally, such prompts for clinical documentation guide the clinician to appropriate clinical documentation based on the patient's status.

According to another aspect of the present invention, an embodiment may include a dependent drop-down. A forms utility includes options for data types, some of these options include: single line alphanumeric, multi-line free-text, check boxes, and drop-downs. The drop-down data type provides an ability to define possible selections from the drop-down list box. The utility provides an ability to map each selection in the drop-down list box to another selection in another drop-down list box. For example, the healthcare organization may define in the utility a data element drop-down list box titled “Review of Systems.” The initial drop-down may include the following selections: HEENT, Gastrointestinal, Circulatory, etc. The healthcare organization may define another data element called “HEENT” which is also a drop-down list box that is dependent on selection of the HEENT option from the “Review of Systems” data element. The selections available in the HEENT may include: Head, Ears, Eyes, Nose, and Throat. Similarly, each selection within the HEENT data element may have dependent data elements. The dependent drop-downs facilitate patient documentation by guiding the clinician to the appropriate documentation according to previous entries.

According to another aspect of the present invention, an embodiment may include a mechanism capable of incorporating multi-disciplinary centralized clinical documentation and sorting/filtering options. During the definition of a data element or set of data elements in the forms utility, the healthcare organization can define the discipline to which the form should be associated. The system incorporates multi-disciplinary use—any clinician can use the system, e.g., physicians, nurses, pharmacists, respiratory therapists, etc. Thus, since all clinicians perform patient documentation, categorizing the forms created by the healthcare organization facilitates electronic searching during documenting and retrieving of patient data. Through this feature, security access of patient entry, editing, and viewing by discipline and/or role type can be provided.

A further categorization titled sub-discipline may also be included in the forms utility. The sub-discipline is defined by the user, e.g., healthcare organization, and is specific to a discipline. This again provides easier, faster searching mechanisms for entry and viewing of data while allowing system security access accordingly.

According to another aspect of the present invention, an embodiment my include healthcare organization definable calculated clinical documentation based on previous patient history. During creation of data elements and choosing a single line edit option with a value type of integer or decimal or when a numeric value is defined for a drop-down list box or check box selection, the system utilizes a forms utility to allow the user to define a calculation to be entered around the value using a mathematical operator. The user-defined calculations include, but are not limited to: addition, subtraction, multiplication, and division. When creating the calculation, a list of all existing integer or decimal data elements can be retrieved for selection of one or more to be used in the calculation. Placement of the selected integer or decimal data element will be at the location of the cursor when the retrieval is made. The user may also cut and paste into the appropriate location, if required. A time tolerance may be defined for the data element. If no time tolerance exists, all values required for the calculation should be recorded in one session at one time to ensure the accuracy of the calculation. The combination of results obtained from various times without consideration of a tolerance may provide an inaccurate calculated result.

According to another aspect of the present invention, an embodiment may include a mechanism to calculate totals for flow sheets. For viewing multi-disciplinary results, the system provides the user with the ability to select a manner and data to total. This accommodates horizontal totaling for each data element and vertical totaling for combinations of data elements. Certain patient documentation requires the addition of data elements to provide a meaningful result. For example, a pain assessment is comprised of several data elements such as pain rating, motor response, motor strength, level of consciousness etc. Although each value independently provides a clinician with useful information, the total pain score is determined by adding each of the data elements with optional weighting values differently from each other. This total score is then used for trend analysis and total pain management. A similar technique can be utilized for input and output documentation. The system's ability to provide this feature dynamically facilitates the clinician's ability to assess patient status quickly and accurately.

According to another aspect of the present invention, an embodiment may include a mechanism to monitor or track the history of user changes to patient clinical documentation. Since patient clinical documentation is a component of the patient's electronic medical record, it is necessary to maintain all changes to the record in complete detail. If patient documentation was completed and then edited due to an error in entry, all history of the initial entry including the replaced entry should be kept as history in the patient's electronic medical record. The present invention provides the ability to edit clinical documentation after entry in specified user-defined circumstances. If editing is performed, the following data is maintained and the new entry is marked as the “Replaced” or new entry: date/time initially entered; date/time edited data was entered; date/time patient was assessed; personnel name entering initial data; personnel ID entering initial data; personnel name entering edited data; personnel ID entering edited data; all data elements entered; and, all data values entered into data elements. The previously entered data can be viewed by querying the patient's record. If a result was edited, the system only shows the most recent entry. If a result was replaced with a new result—through the editing feature—the detailed results viewing window will display all entered results identifying which were edited results and which were newly recorded results.

According to another aspect of the present invention, an embodiment may include user-definable views of entered patient clinical documentation. Although structured clinical documentation through forms differ from free-text clinical documentation, it is often desirable to view a patient's total results—both noted documentation and monitoring parameter/lab/diagnostic result reporting together or independently in chronological or reverse chronological order. Therefore, another aspect of the present invention provides for viewing all results for a patient within free-text clinical documentation. When structured results, other than clinical documented notes, are viewed from the clinical documentation screen, the format of display is a text blob in which the data is separated by a pipe, |. The information is not stored in free-text clinical documentation, but is instead retrieved for viewing purposes only. The results are viewed from the multi-disciplinary results window for trend analysis and graphing.

During system configuration when creating clinical documentation categories and sub-categories, the system provides the healthcare organization with the ability to retrieve result name(s) from any discipline. More than one result name may be attached to a category definition. For example, a category titled “Nursing Progress Notes” may be defined and within this category, the user may create sub-categories—Subjective, Objective, Assessment, and Plan. The subjective, assessment, and plan categories can be defined as free-text entry documentation categories; whereas, the objective sub-category may include heart rate, blood pressure, respiratory rate, and temperature, along with particular serum chemistry. The specific monitoring parameters or test results listed above for the objective sub-category are typically part of structured clinical documentation. The audit trail/history of the creation of these category definitions is maintained.

Within the system, a utility provides Rules to allow the healthcare organization to determine where clinical documentation elements/results are capable of being viewed, i.e., from the multi-disciplinary view results area and/or the free-text clinical documentation area. When viewing free-text clinical documentation, the structured clinical documentation categories/sub-categories can appear along with the regular hospital-defined categories. This allows the clinician to view results entered in a structured format while simultaneously viewing free-text patient documentation.

According to another aspect of the present invention, an embodiment may include a mechanism to copy previous entry functionality. In a forms utility, healthcare organizations are provided with an ability to allow clinicians to copy values from the previous entries into the current documentation. This rule is defined at the lowest data element level, as well as at the set and group levels. When performing clinical documentation, clinicians can view previous relevant associated entries while simultaneously completing current documentation. If copying is allowed, the data element entry from the previous entry will default into the current documentation field when the user indicates by clicking a “Copy from Previous” hyperlink. The clinician may edit the entry prior to saving.

According to another aspect of the present invention, an embodiment may include a mechanism for scheduling patient documentation associated with medication administrations. Users, preferably healthcare organizations, are provided the ability to associate a patient's clinical documentation with a medication administration. The healthcare organization may indicate that one or more patient data must be collected around the medication administration process through the use of the hospital-definable forms. When defining the elements to be captured around the medication administration, the healthcare organization defines the time at which the data should be collected. For example, if a pain medication is being administered, a pain assessment can be scheduled before and after administration. The healthcare organization can also define whether data documentation is mandatory or optional, and whether a reason code is required for non-entry. Once all rules are defined, healthcare personnel can be alerted to perform clinical documentation according to the medication administration schedule. The alerts can be received on a regular workstation, a tablet, a laptop computer, and/or a PDA (i.e., personal digital assistant). This aspect provides workflow management and decision support at the point of care while ensuring patient clinical documentation.

To further assist in the understanding of the aforementioned aspects of the present invention, an exemplification of a particular embodiment of the present invention is discussed in further detail below. It is to be understood that the present invention is not limited to this particular embodiment and that any number and combination of the aforementioned aspects may be incorporated into a given embodiment. With respect to the following exemplar embodiment, text shown in bold font indicates fields, soft keys, functions, radio buttons, menus, screens, etc., that are viewable on a display of a device operably connected to the system of the present invention, such as, for example, but not limited to, a PDA, tablet or workstation.

A login window is presented to enter a new User ID and Password. The user, e.g., physician, logs into the Computerized Physician Order Entry (CPOE) application by entering a User ID and Password. If the User ID and Password is invalid, an error screen will appear stating such and the user will be prompted to re-enter the User ID and Password. A related security feature is activated when a predetermined period of inactivity is exceeded. The security feature will cause the application to automatically sign a user off the application and present the user with the Sign On window. The length of inactivity before a user is signed off is determined by a facility utilizing the application.

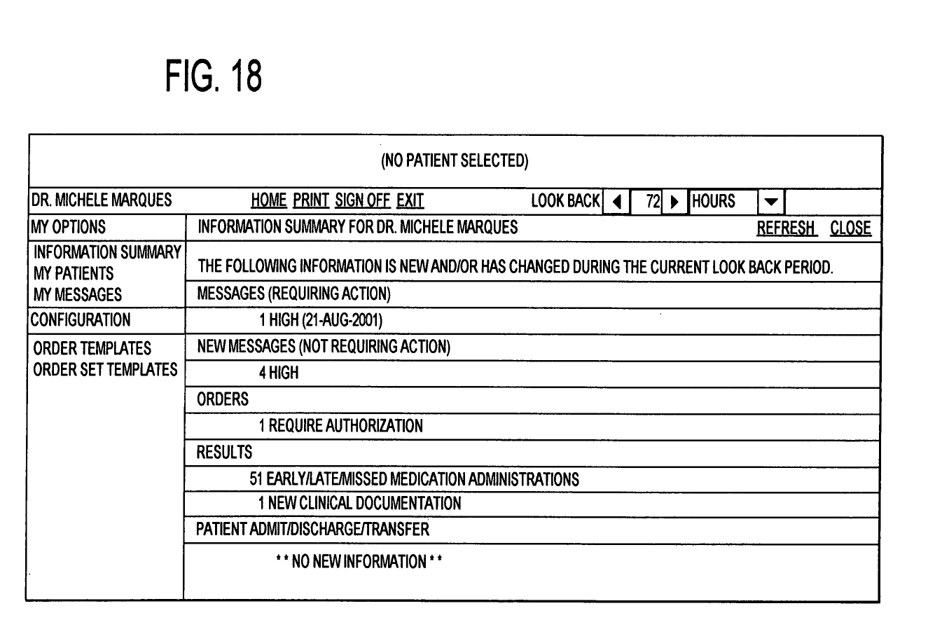

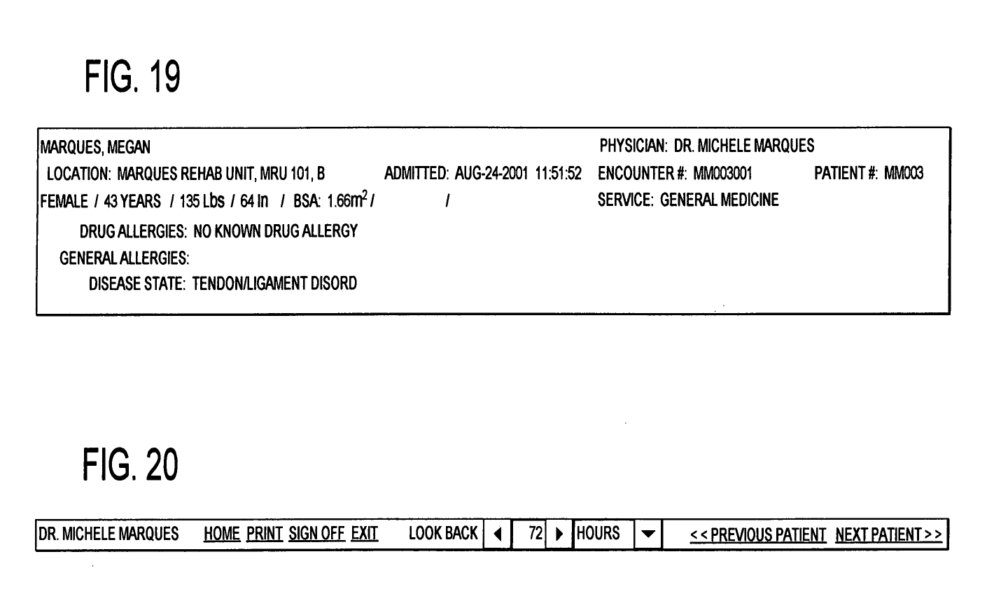

Upon logging in, a main screen will be displayed (See FIG. 18). The top portion of the main screen is the Patient Information Panel (See FIG. 19). The initial screen display does not include a patient. Once a patient is selected, this section provides a summary profile of the patient and the current encounter. This is a view-only section and provides demographic information only.

Below the Patient Information Panel is the Navigation Bar, which is preferably available on all screens (See FIG. 20). The left side of the Navigation Bar displays the current user's name. Other functions on the navigation bar include:

Home—takes the user to a Physician Information Summary screen;

Print—allows the user to print the current window contents;

Sign-Off—signs the current user off of application;

Exit—exits the application;

Previous/Next Patient—a selector is displayed only once a patient has been selected; from any screen displaying patient information, this selector changes the current patient to the previous or next patient, based on the contents of the My Patients list;

Look Back—a period selector controls how much data is displayed in the current window, based on date and time; the look back period selector has a memory, meaning that its value is remembered based on a user's ID. Upon subsequent sign ons, the Look Back period will default to the last period the user requested.

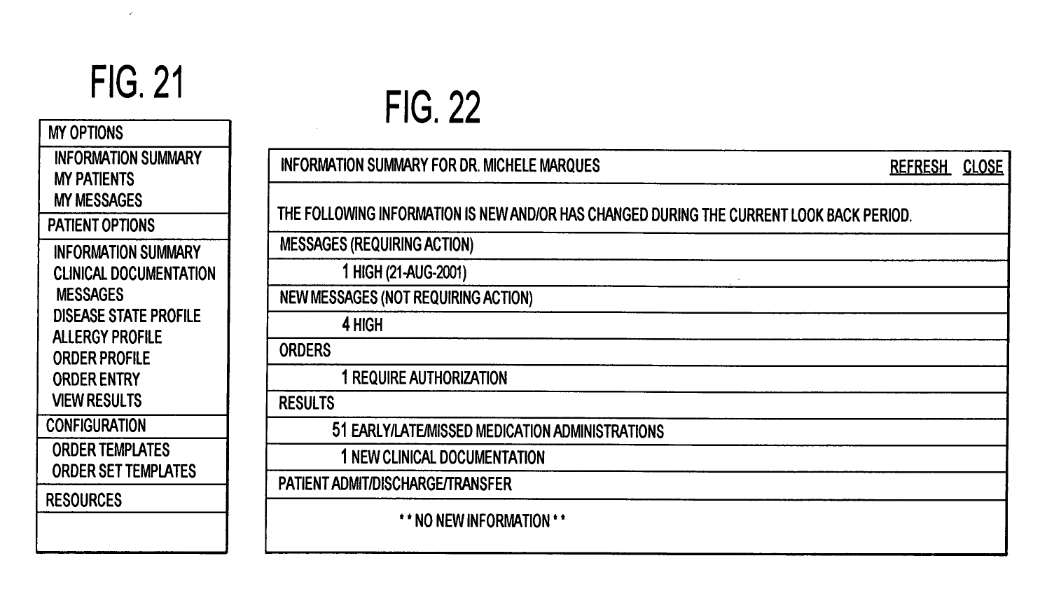

All available user options are available on the Menu Panel located on the screen. Until a patient is selected, this section only displays MyOptions and Configuration. Once a patient has been selected, this section displays My Options and Configuration, along with the addition of Patient Options and Resources (See FIG. 21). These section assist with navigating through the application and providing the user with access to specific patient information.

Upon logging in, a personal information screen, also called the Physician Information Summary screen, is presented to the user (See FIG. 22). This screen contains a summary of all relevant information and information relating to a physician's patients that has changed. The Lookback Period selector may be used to limit or expand the amount of information displayed. The information is displayed in the following order:

Messages (Requiring Action) summarizes messages requiring action and displays each priority and their respective number of messages requiring action. The display is independent of the look back period. Messages requiring action appear regardless of the look back period selected. Messages not yet acted upon, whether or not falling within the look back period, are included here. The date of the oldest occurrence will appear in brackets next to the priority of the message.

New Messages (Not Requiring Action) summarizes new messages not requiring action in order of priority (Stat, High, Medium, Low, Missed). The number of messages is displayed beside the priority.

Orders summarizes the amount of actions/reviews required for orders on a physician's patient list. This section is divided into orders requiring authorization; orders that were authorized by an alternate physician in the last few days which may need to be reviewed by the attending physician; and orders that have expired within the last few days or those that will expire within the next few days.

Results presents exception-based results according to flags sent by the laboratory and diagnostic systems interfaced with the application. Any abnormal results received from ancillary systems to which the application has been interfaced will also be returned. Under abnormal results, the number of early/late/missed medication administrations is displayed. Clinical Documentation includes any new clinical documentation.

Patient Admit/Discharge/Transfer includes information regarding the number of patients admitted, discharged or transferred. Beside each item is a number. By clicking this number, a user will go directly to the My Patients list, Order Profile screen, or Results Viewing or Messages screens, and display the appropriate detailed information. Each of these screens can also be accessed through the menu located on the screen.

The application includes e-mail for communicating with other users of the system. Messages sent by others working at the facility, such as physicians, nurses and pharmacists, can be received. In addition, automatic alerts concerning a patient can be utilized to notify about events concerning a patient.

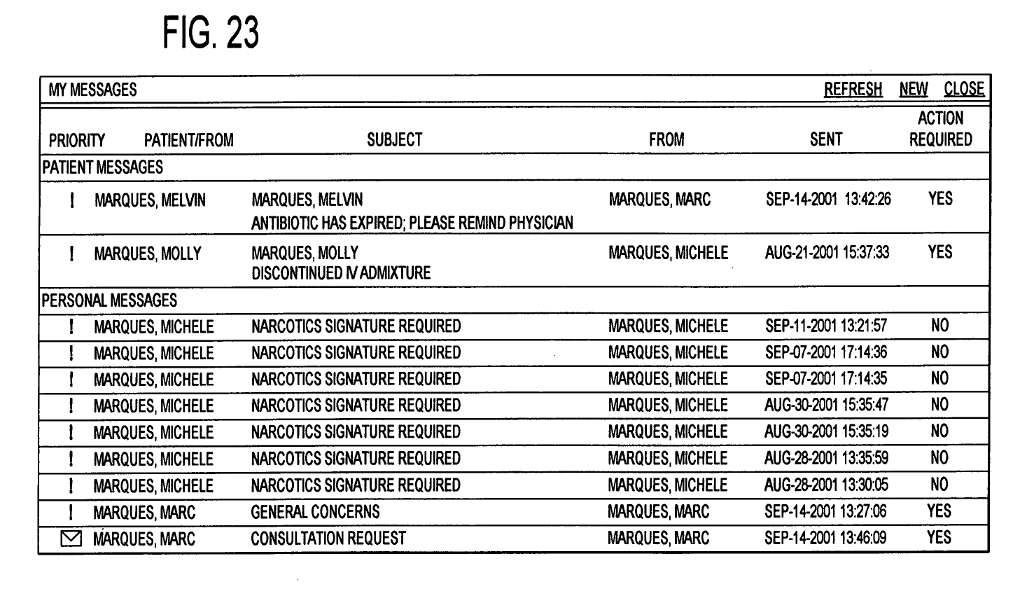

A Message Summary screen displays messages that relate to a physician's patients or that have been directed to the user (See FIG. 23). The list is grouped into two categories: Patient Messages and Personal Messages. The messages can be sorted by clicking on the column name by which the data is to be sorted. The My Messages screen has column headings for: Priority, Patient/From, Subject, From, Sent and Action Required. Priority refers to the priority level of the message. An envelope icon represents a message of low priority, a yellow exclamation mark icon represents a message of medium priority, and a red exclamation mark icon represents a message of high priority.

The Patient/From in Patient Messages lists the names of the patients related to the messages. In Personal Messages it lists the names the messages are from. The Subject field in Patient Messages lists the names of the patients and a brief indicator of what the message is about. In Personal Messages, only the subject of the message is listed. As is typical, From indicates who sent the message, and Sent lists the dates and times the messages were sent. Action Required indicates either Yes an action is required, or No action is required.

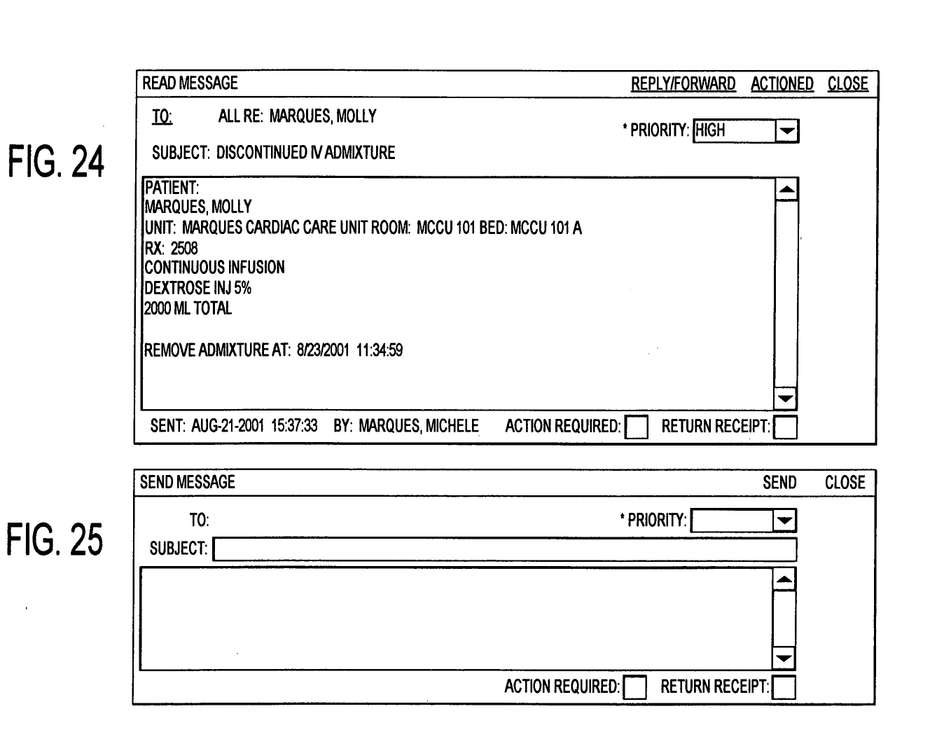

To read the message, click on either the patient name in Patient Messages or the name of the person the message was From in Personal Messages (See FIG. 24). In the Read Message window, one may choose to reply to or forward the message. Click the Actioned link if the action required has been completed; delete the message or close the Read Message window. Actioned will not appear if no action is required. Note—at the bottom of the Read Message window, the date and time the message was sent is indicated as well as by whom it was sent. If an action is required regarding the message there will be a checkmark in the Action Required checkbox and if a return receipt is required there will be a checkmark in the Return Receipt checkbox.

To send a new message, click on New at the top right hand corner of the My Messages screen (See FIG. 25). Click on To in order to get a listing of personnel from which and addressee can be chosen. The priority level of the message is selected by clicking on the downward pointing arrow. The subject heading can be filled out. The large blank box is the free-text area in which the message can be placed. At the bottom of the window, you may choose to click on the box for Action Required or Return Receipt if these options are necessary. Click on Send when your message is complete and the message will be sent to the personnel indicated.

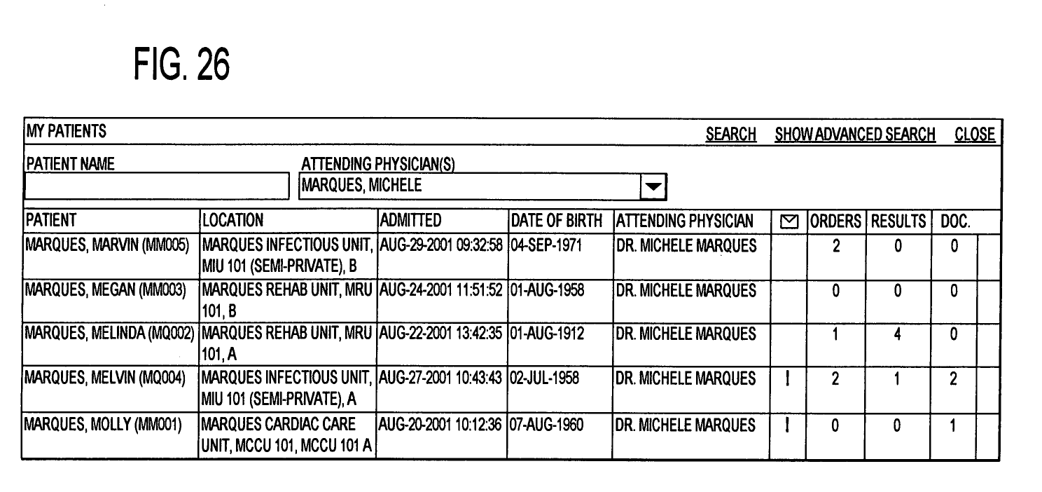

The My Patients screen lists basic demographic information about a physician's patients (See FIG. 26). By default, this lists all patients with active encounters for which the logged-on user is the attending physician. Other patients can be added to the list by searching for them. At the top of My Patients screen, search can be performed by Patient Name, and/or Attending Physician. The Show Advanced Search option will make the Encounter Locator and Patient Locator search fields visible. The Encounter Locator and Patient Locator fields can be hidden by selecting Hide. When a physician is selected, if the user is signed on as a physician, the Attending Physician(s) selection will already have that name highlighted. Further attending physician(s) from this list can be chosen by highlighting the physician(s) name. Multiple physicians can be selected at once. When a search is performed, the resulting patients shown will form the My Patients list. This list will be used when navigating between patients on the Patient Information section, at the top of the screen. Once the patient list is defined, the application will remember this list for the user until changed by the user or the user logs off. The My Patients list displays Patient Name, Location, Date Admitted, Date of Birth, Attending Physician, outstanding messages indicator, orders requiring authorization indicator, abnormal results indicator, and new clinical documentation indicator. The patient list can by sorted by any column header simply by clicking on the appropriate column name. Click on a patient to make that patient the currently selected patient. The Patient Information Summary screen will be displayed for the selected patient. In addition, the patient's demographic information will be displayed in the Patient Information panel at the top of the screen. From the list of patients, one can go directly to specific information about the patient, i.e.:

Click on the outstanding messages indicator to view patient specific messages in the Patient Messages screen;

Click on the new/changed orders indicator to go to the Order Profile window;

Click on new results indicator to go to the View Results window; and,

Click on new clinical documentation indicator to go to the Patient Clinical Documentation window.

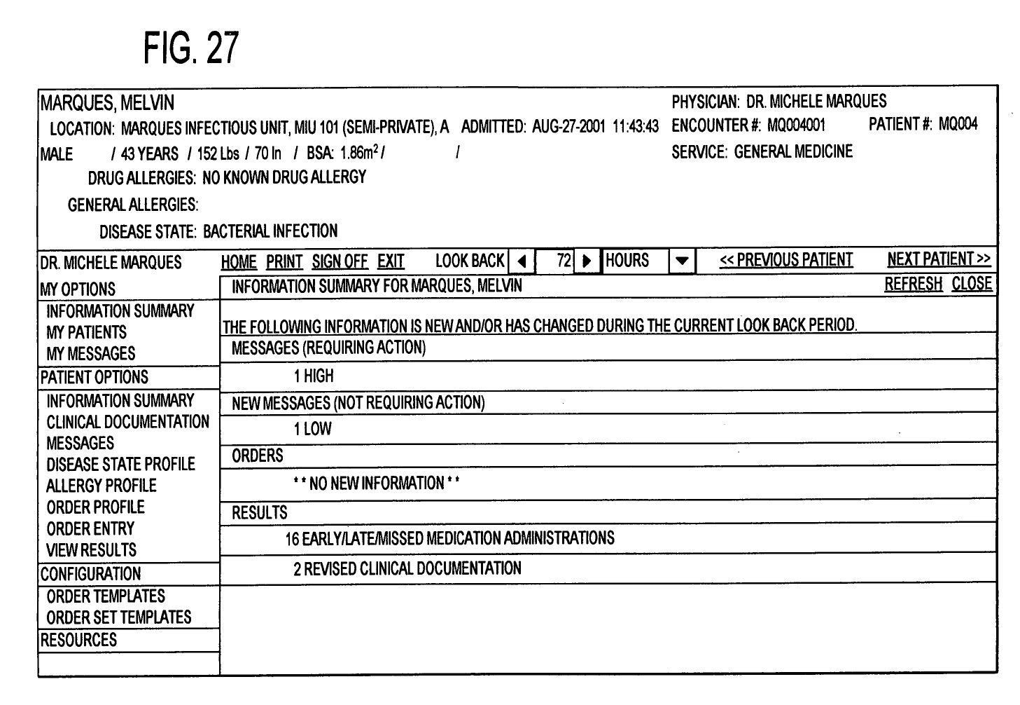

The Patient Information Summary page contains a summary of all relevant information and information that has changed about the selected Patient since the date and time defined by the Lookback Period selector (See FIG. 27). Preferably, the relevant information includes:

Messages (Requiring Action) lists messages requiring action in order of priority, e.g., Stat, High, Medium, Low, Missed. The number of messages is displayed beside the priority.

New Messages (Not Requiring Action) lists messages not requiring action in order of priority, e.g., Stat, High, Medium, Low, Missed. The number of messages is displayed beside the priority.

Orders lists any orders that require authorization, orders authorized by another physician, orders that expired and orders that will expire.

Results will also list abnormal results, early/late/missed medication administrations and any New or Revised Clinical Documentation.

Clicking on any of the status items will navigate the user to the Order Profile, Results Viewing, Messages, or Clinical Documentation windows.

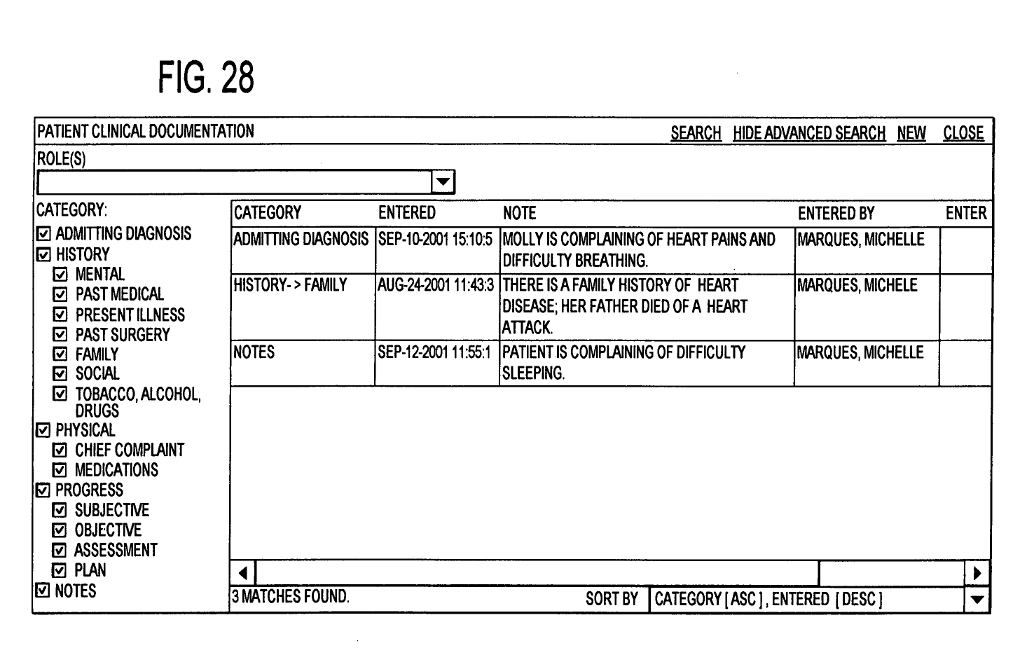

Patient Clinical Documentation is a dynamic feature that enables physicians and other healthcare resources to write documentation pertaining to a patient and/or encounter in a transaction-oriented environment (See FIG. 28). All documentation transactions are time stamped. Documentation may be created and continued, but existing documentation cannot be changed.

Sort and Filter Documentation includes categories in which clinical documentation can be organized. The categories must include, but are not limited to: History, Physical, Progress, Notes, and Admitting Diagnosis. A facility may define additional main categories and subcategories. To view all categories, ensure that all checkboxes have a checkmark. A category can be de-selected by clicking on the main category checkbox to remove the checkmark. All subcategory check marks will simultaneously disappear. To de-select a particular subcategory, click on the check box next to the specific subcategory name—which will remove the checkmark for that subcategory only.

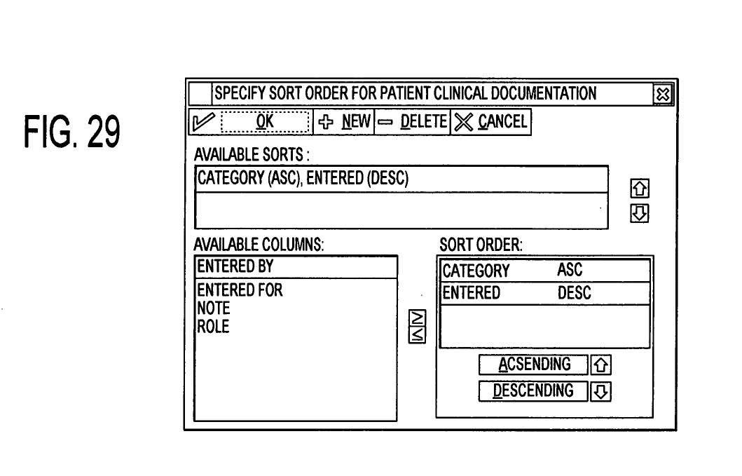

The documentation list can be filtered by Role(s). When one or more roles are selected, only results recorded by people in those roles are displayed. For example, only documentation recorded by physicians can be displayed. A Hide Advanced Search feature will hide the category search criteria, which allows more viewing room for the documentation. The information displayed on the clinical documentation screen may be sorted by clicking on the column name for which the data is to be sorted. Custom sort orders can be created by utilizing the Sort By selection at the bottom of the screen. When Sort By is selected, a screen appears that lists Available Sorts and Available Columns (See FIG. 29). This screen allows the creation of a Sort Order. Descending and ascending options are also available. To create a new Available Sort, click New. To delete an available sort, click Delete. When finished, click OK to apply any changes. Once the Selection Criteria process is complete, the user can view the clinical documentation according to what has been selected. The Patient Clinical Documentation screen displays the categories selected, the date and time the note was entered, the first line of the note, who entered the note, and the role of the person who entered the information.

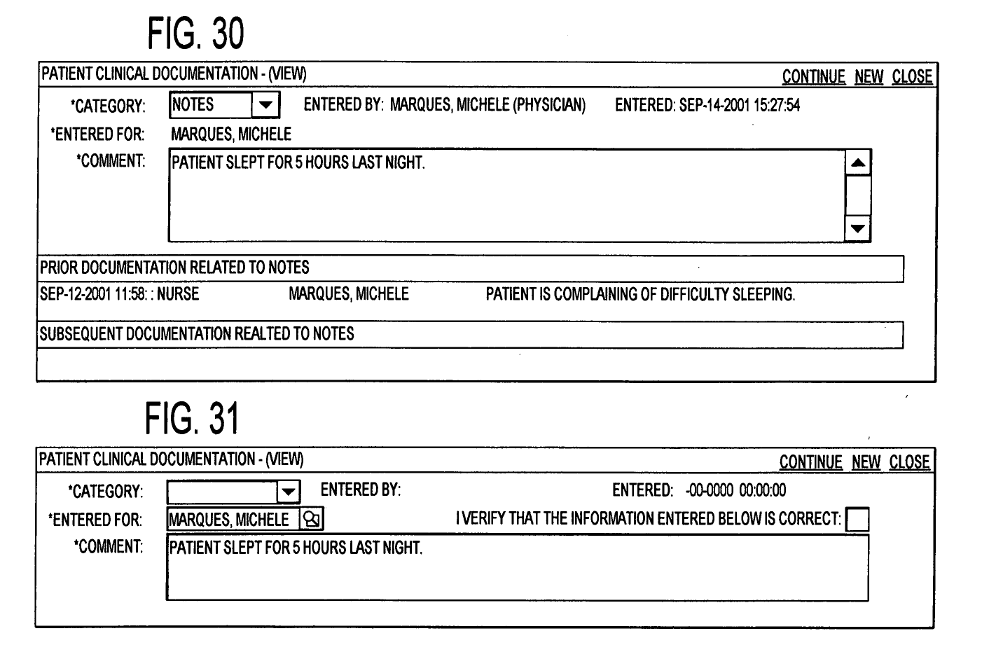

By clicking on any one of these categories, a Patient Clinical Documentation—(View) screen will appear (See FIG. 30). This screen displays additional details about the documentation, including previous and subsequent documents.

In order to create new documentation, click on New at the top right hand side of the screen. Select from one of the predetermined categories (See FIG. 31). Entered for defaults to the user signed on. If information is entered on behalf of someone else, select that person. Enter the information in the blank Comment box. Entered by will default to the user's name and the date is time stamped automatically. Click Save when documentation is complete.

The Edit screen also enables the entry of information in Prior Documentation and Subsequent Documentation related to the category specified. Click Continue in order to enter further documentation pertaining to the comments entered directly above this option. A new patient clinical documentation window opens with the category set to the category chosen for the previous documented note. Once this new document is saved, the original clinical documentation will appear as a hyperlink under the section titled Prior Documentation related to “Category Name.” This functionality allows a clinician to easily follow a patient's status concerning a particular issue.

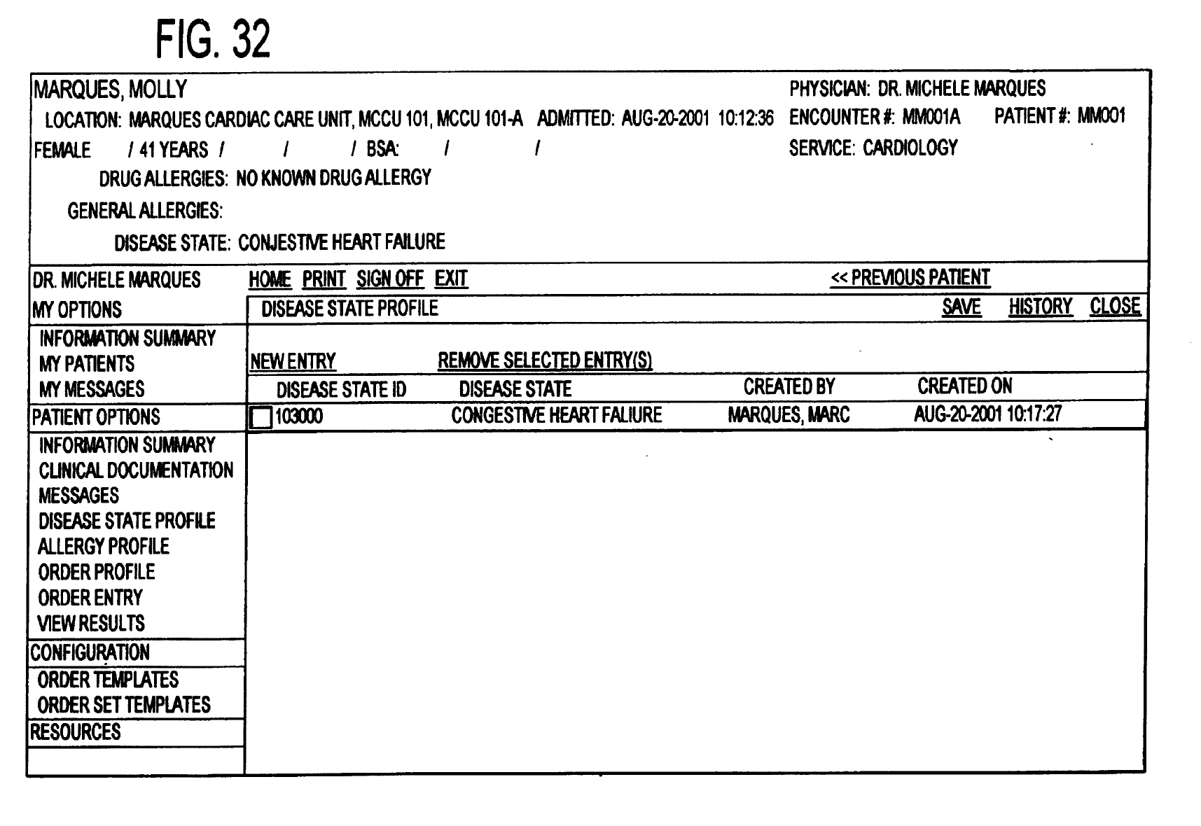

A Disease State Profile is included in the selected patient's profile and displays the disease state(s) (See FIG. 32). The profile includes: the Disease State ID number, the Disease State, and an audit trail including the identity of personnel who entered the disease state onto the patient's profile along with time it was entered and/or modified, when relevant. When the Disease State Profile option is clicked, the active window displays the current disease state profile for the selected patient. This information is the same as that displayed in the patient header demographics.

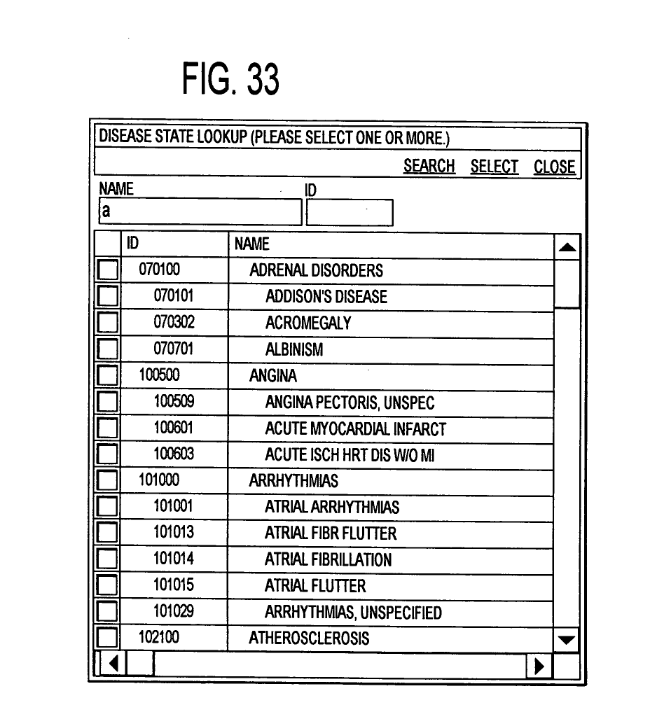

To add a new disease state to a patient's profile, click New Entry above the list of disease states. A Disease State Lookup screen will appear (See FIG. 33). This screen facilitates searching a pre-defined list of disease states. To search, enter in the first few characters of the disease state or enter in the disease State ID number and click on Search or hit the <Enter> key on the keyboard. A list of disease states will appear according to the search criteria.

A disease state can be chosen from the search result list by clicking on the checkbox beside the disease state wanted, then clicking on Select. This will transfer the disease state selected onto the patient's profile. In order to save this information, click Save. This is now the patient's new current disease state profile and the patient header demographics will reflect this.

To delete a disease state from the Disease State Profile, click on the checkbox beside the disease State ID number and a checkmark will appear. Click Delete on the top right hand side of the Disease State Profile screen. Multiple disease states can be selected and deleted. A disease state history can be viewed by clicking on History at the top right hand corner of the Disease State Profile screen.

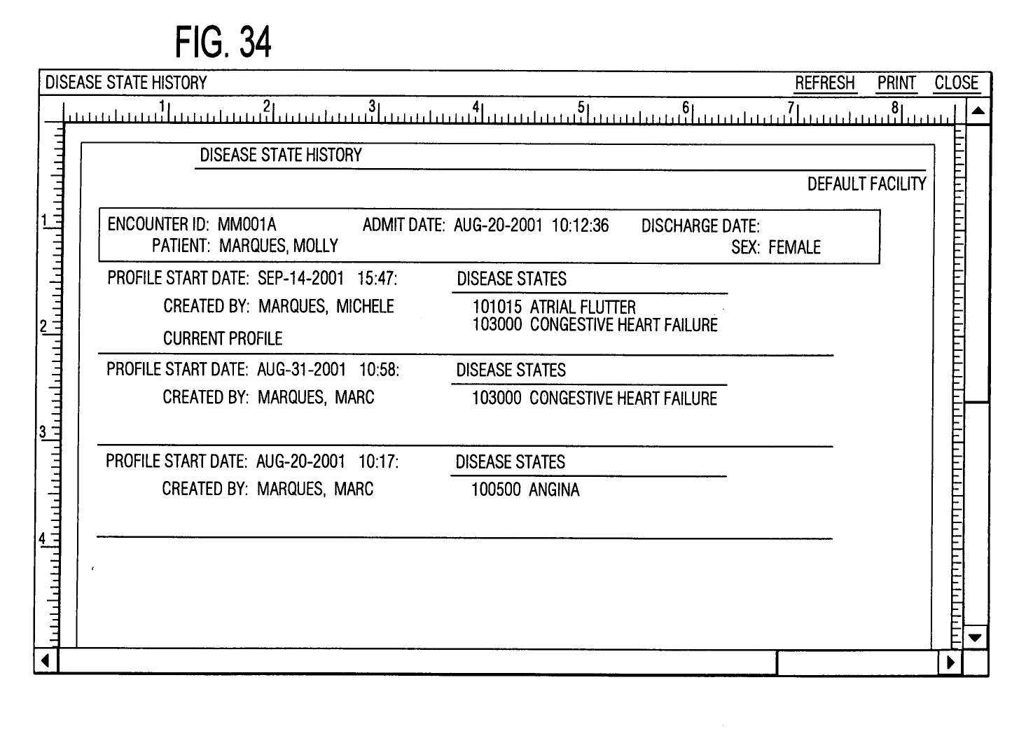

The top of the Disease State History screen shows the patient demographic information. (See FIG. 34). The disease state history is listed in chronological order starting with the patient's current profile. Details such as profile start date and time, personnel by which the profile was created, and disease states are listed. A printout of the disease state history can be obtained by clicking Print.

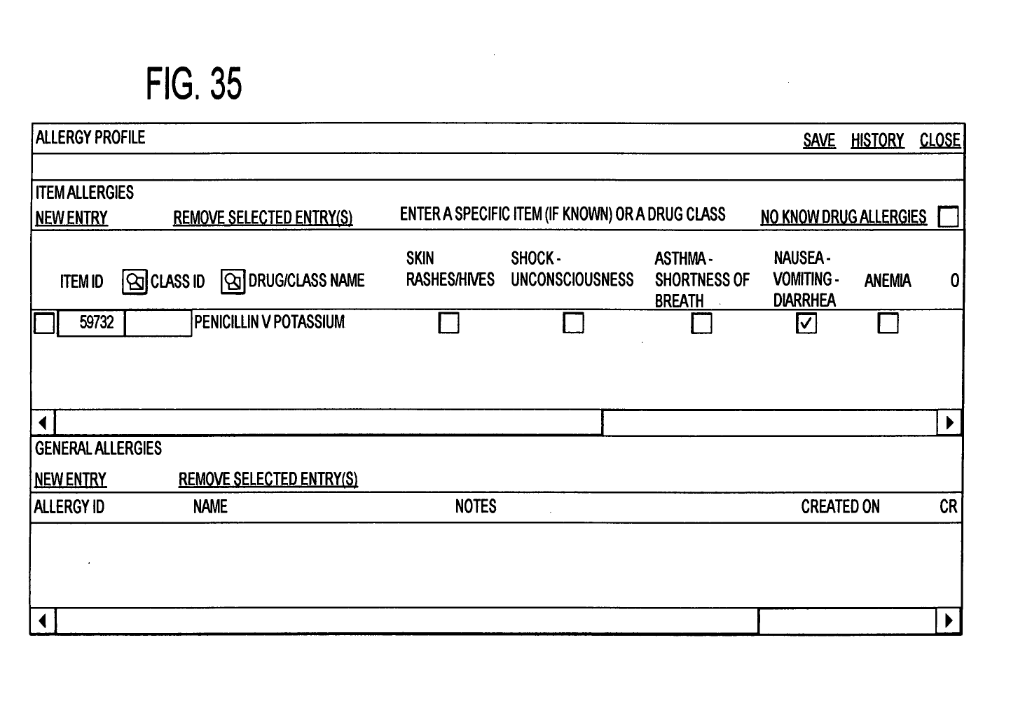

Clicking Allergy Profile on the left side of the screen will display the selected patient's current allergy profile. (See FIG. 35). An audit trail is displayed of each allergy entered along with the personnel who entered the allergy, the time/date it was entered, and if relevant, the personnel who modified the allergy and the time/date of its modification. The allergies appearing will be identical to the allergy profile presented in the patient information panel at the top of the screen.

Orders are screened against item allergies. When an order for an item is placed for which there is a recorded item allergy, notification of the allergy is made. An option is provided to cancel the order or specify an override reason. If there are no known drug allergies, the No Known Drug Allergies check box should be checked. Otherwise, the drug allergies will be listed as unspecified. Notification, e.g., an alert, is provided when an order for a patient with unspecified drug allergies is made. In the Allergy Profile for each item allergy, the window lists the Item ID, Class ID, Drug/Class Name and check boxes for allergic reactions. To change the allergic reaction, check or clear the appropriate check boxes and then click Save.

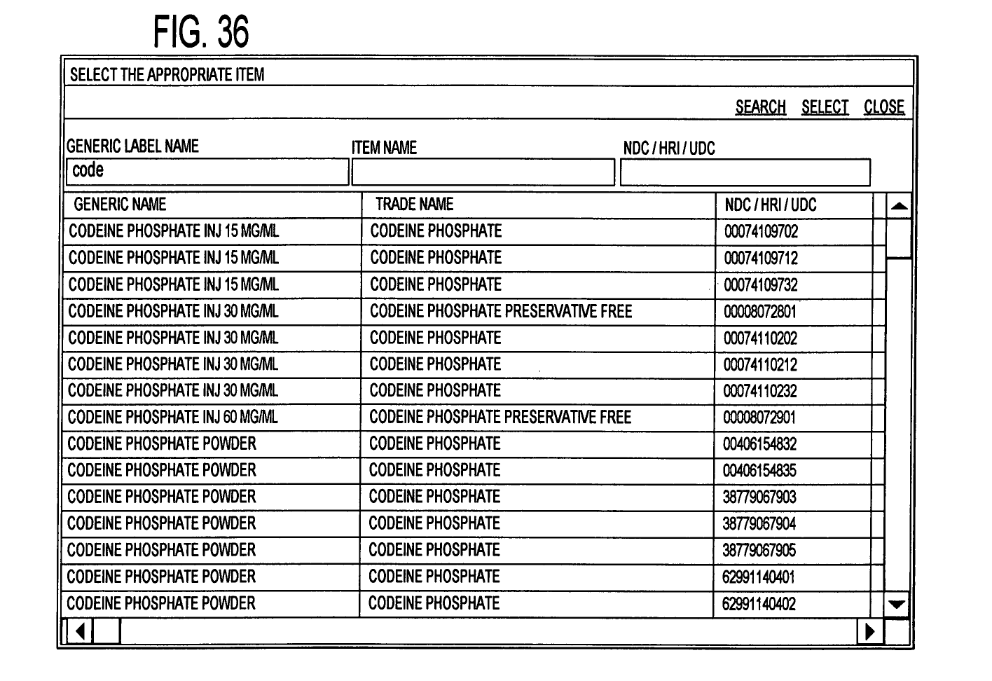

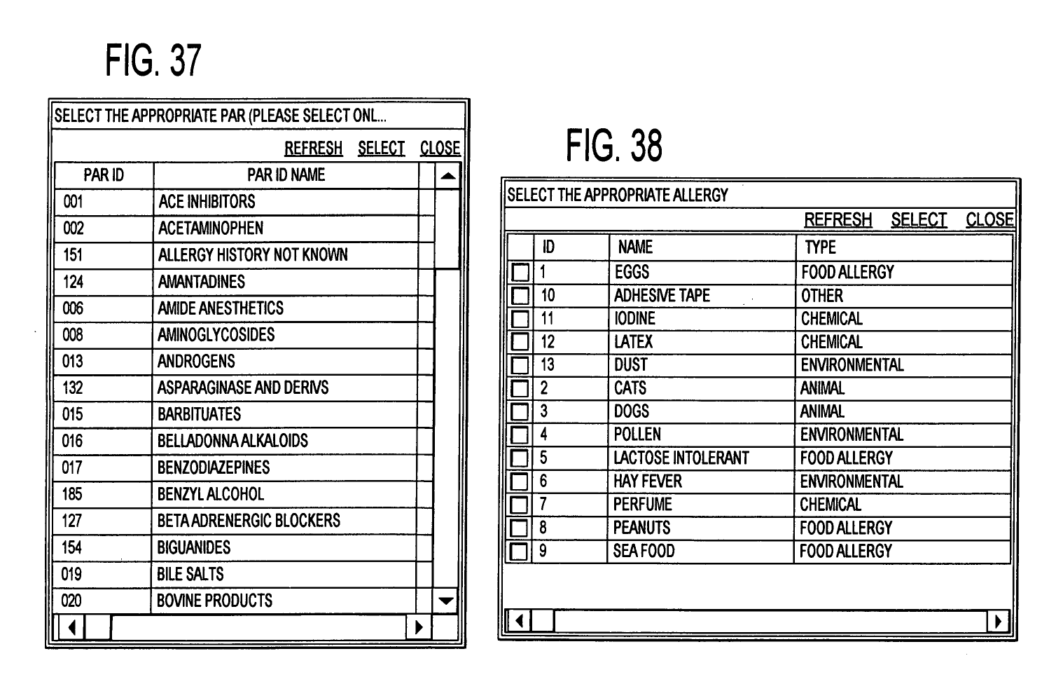

To record a new item allergy, click New Entry. A new row opens that must be completed. Enter either the Item Id or Class ID and check the appropriate reaction check boxes. To select an Item ID, either click the Item ID search button or right-click in the Item ID box. A search window to help you Select the Appropriate Item opens. Enter the first few letters of the generic label name, item name, or NDC/HRI/UDC and click Search. A list of matching items is displayed (See FIG. 36). Click the appropriate item to be selected (See FIG. 37). To select a Class ID, click the Class ID search button and a window opens from which an appropriate class can be selected. After the appropriate reaction check box(es) have been selected, click Save to save the new allergy to the profile. The profile will be listed in the summary at the top of the screen.

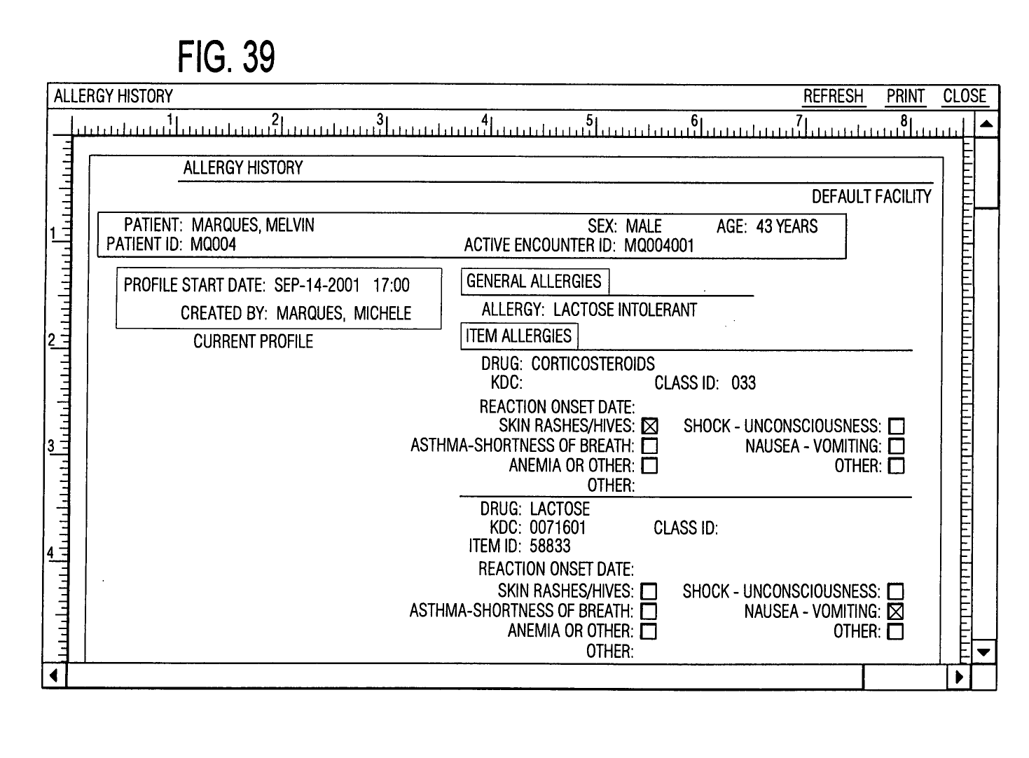

General allergies are recorded for information only. Note—if a non-drug allergen is relevant to ordering drugs, e.g., peanut oil or lactose, it should also be entered as an item allergy. This is necessary as only item allergies are screened against orders. For each general allergy listed, this window displays the Allergy ID, Name and Notes, as well as who created the entry and the time this entry was created. A window opens listing general allergies (See FIG. 38). Click the check box next to each appropriate allergy, then click Select. The list may be sorted by Name or Type by clicking the heading of the appropriate column. A new row is created for each allergy selected. Enter any applicable Notes and click Save. The allergy history can be displayed by clicking the History button (See FIG. 39).

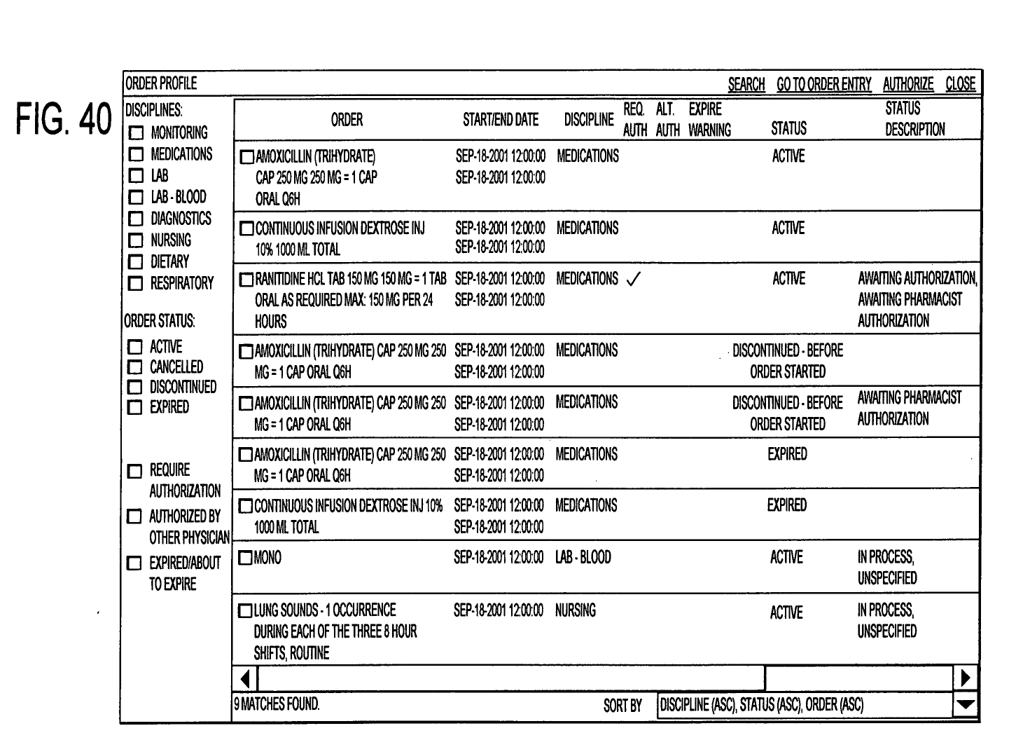

Once a patient has been selected, Patient Options becomes an additional selection option of the menu panel. To view a patient's order profile, click Order Profile. The order profile provides a summary of all orders for the selected patient (See FIG. 40). For each order, the Order Profile screen displays the Order, Start/End Date, Discipline, Status, Status Description, and whether the order Requires Authorization, whether it was authorized by an alternate physician (Alt. Auth), and whether it is about to expire (Expire Warning). Additional information is provided such as whether the order is for an admixture or is a PRN order. Order provides a summary of what a clinician ordered. Start/End Date lists the duration date and time of an order. An end date will not displayed when an end date has not been specified by the clinician at the time of order entry. Status Description will display a brief description when an order is pending approval or requires further action. Discipline refers to the type of order, e.g., Monitoring, Medications, Lab, Lab-Blood, Diagnostics, Nursing, Dietary, Respiratory and Physiotherapy, prescribed for a patient. Order Status indicates whether an order is Active, Cancelled, Discontinued or Expired.

One may quickly sort the list of orders by clicking on the desired column name for which the information is to be sorted. Orders may be filtered by Discipline and by Order Status. By default, the system will display orders for all disciplines and for all statuses. The display can be restricted to the specific disciplines and order statuses that are selected. On the left side of the screen, check the desired disciplines and order statuses to display. If no disciplines or order statuses are checked, then all are displayed.

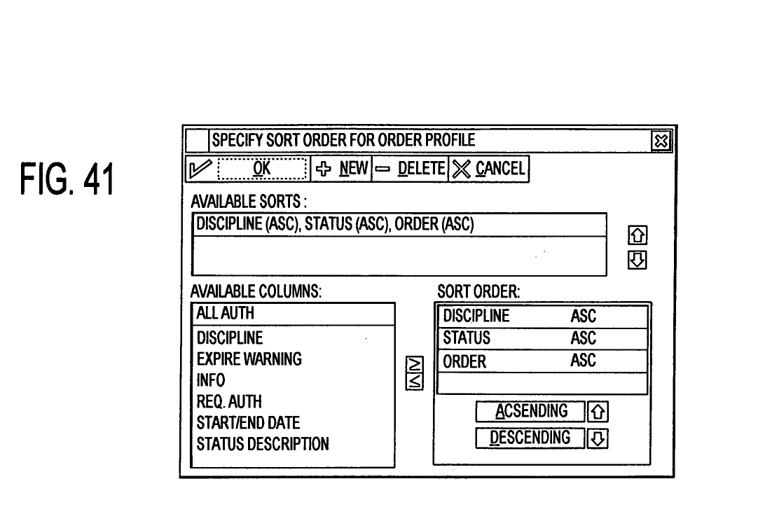

Orders may also be filtered by the flags Require Authorization, Authorized by other Physician, or Expired/About to Expire. These settings will be saved and this view will be present with the selected filters until they are changed. Checking or unchecking Discipline or Order Status, the list will be refreshed to match the new criteria. In addition to quickly sorting by a specific column, a more detailed custom sort can be created. Once created, the custom sort can be used until deleted. Custom sorts can be created from the Sort By selection at the bottom of the screen. By clicking Sort By, a screen will appear that lists Available Sorts and Available Columns (See FIG. 41). From this screen, a Sort Order can be created. Option to list the sort in descending and ascending order are available. To create a new Available Sort, click New. To delete an available sort, click Delete. When finished, click OK to apply any changes. The custom sort can now be selected from the Sort By list.

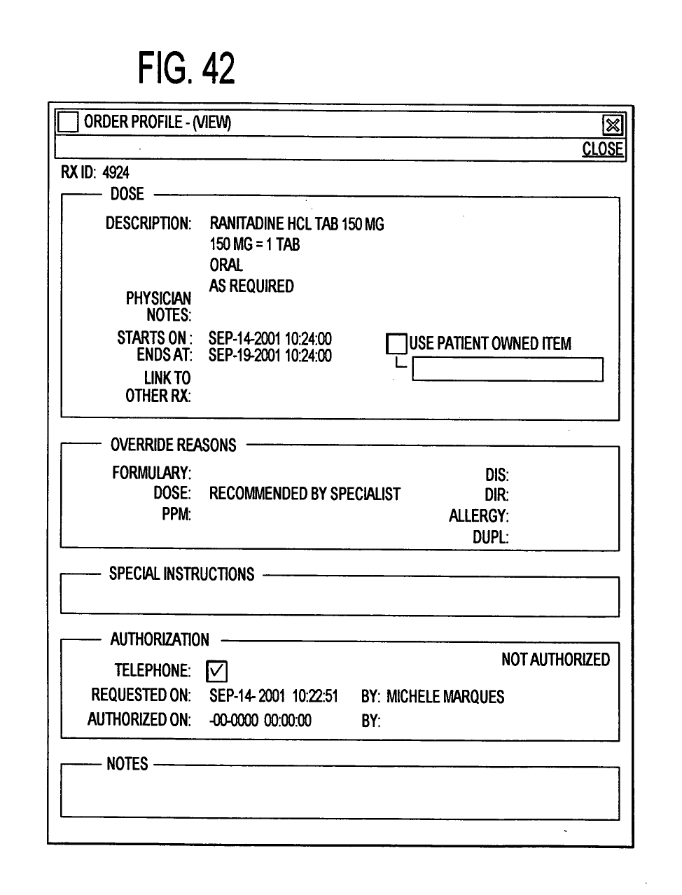

Clicking on an order will open a pop-up Order Detail window showing additional information about the order (See FIG. 42). This window is view only, and will vary depending on the type of order (such as whether a medication or non-medication order is selected).

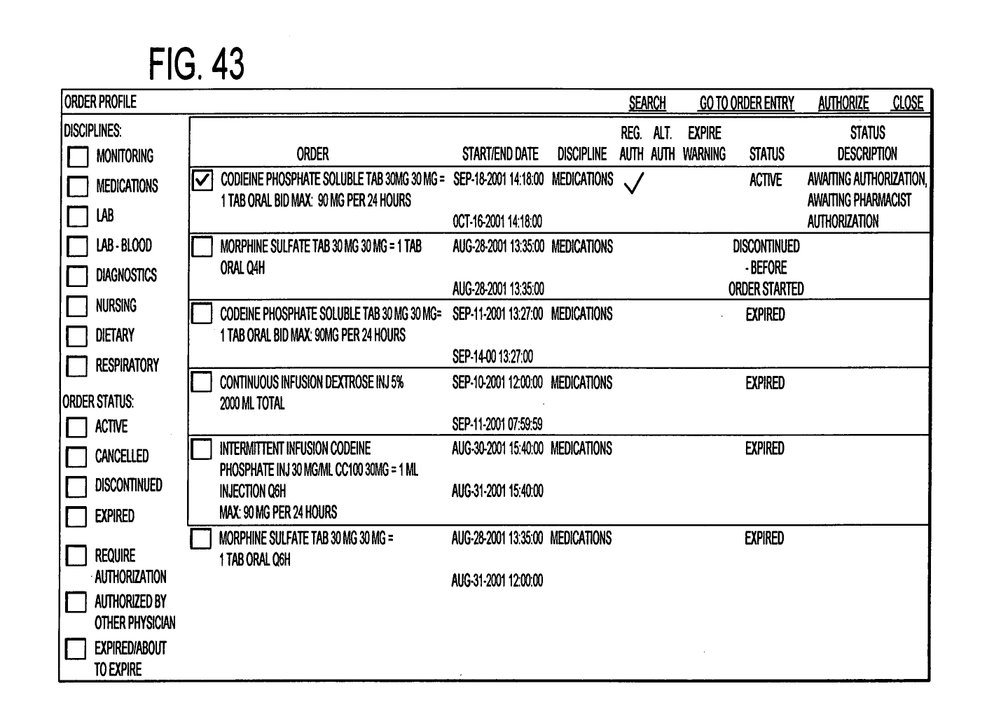

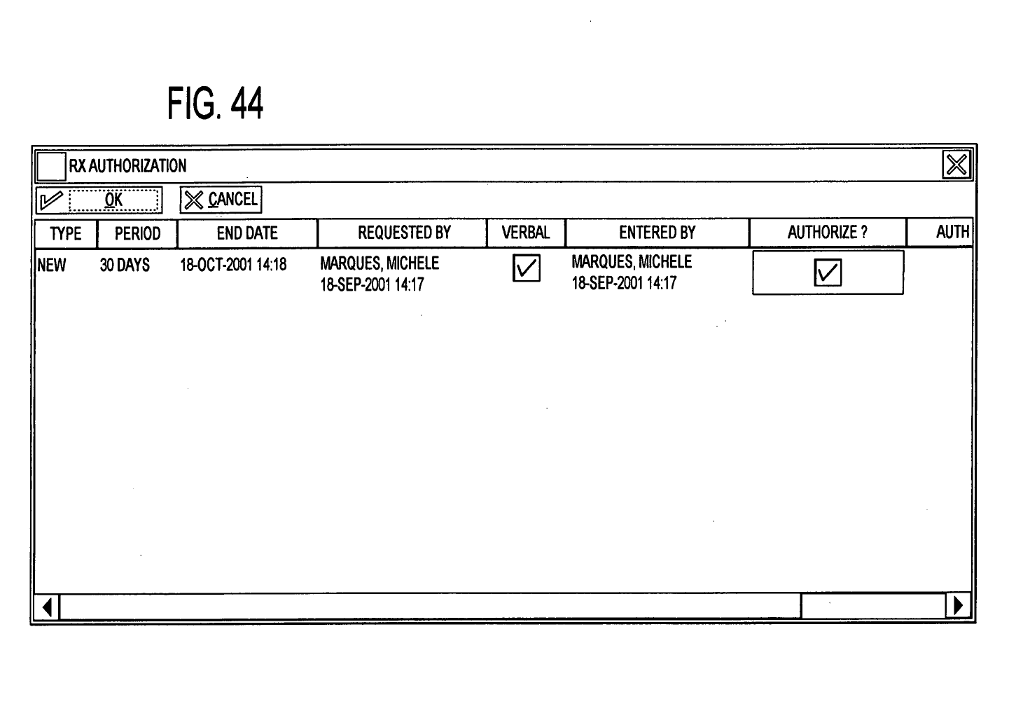

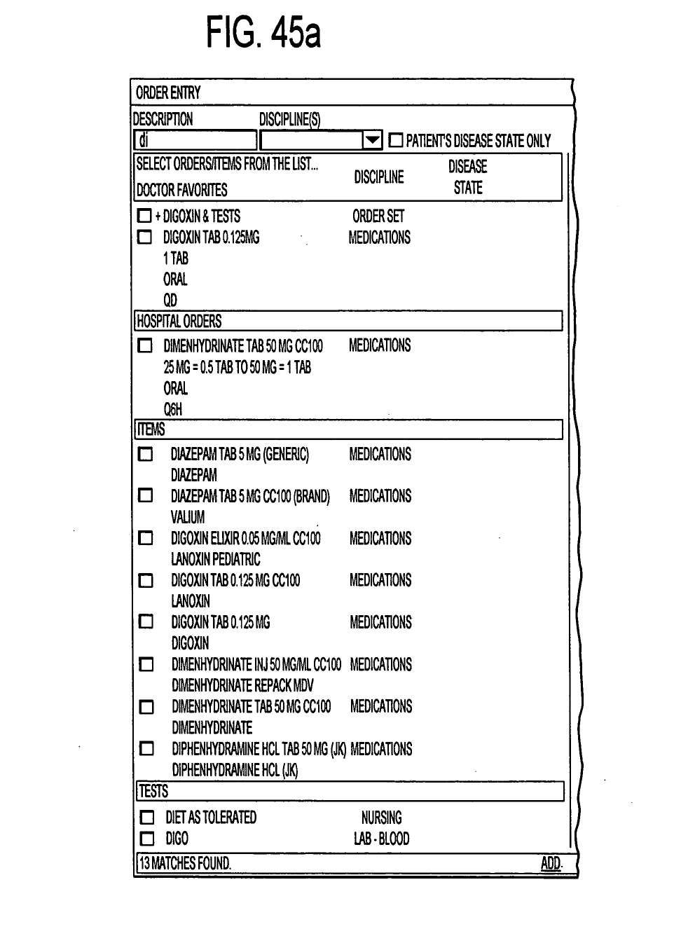

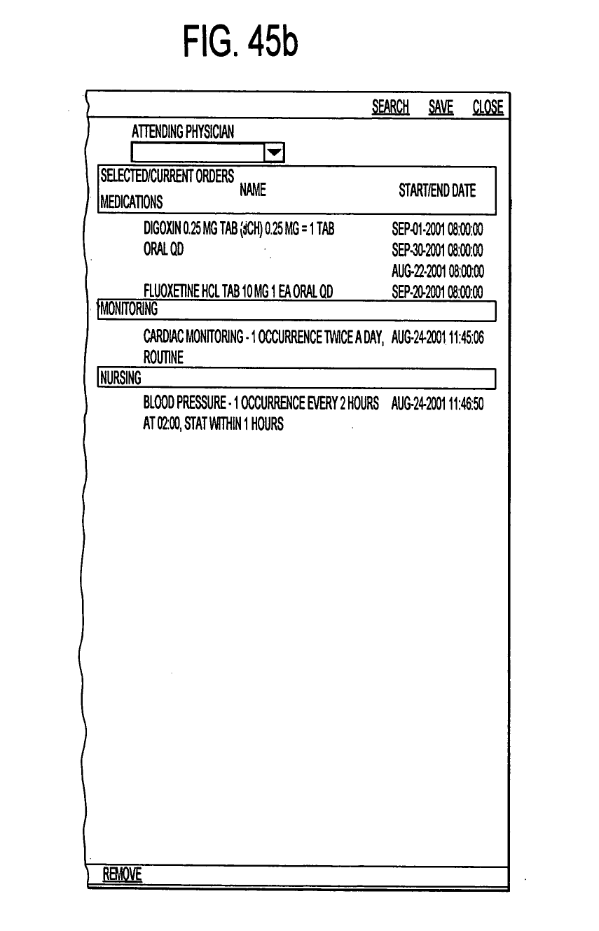

Orders that require a user's authorization have a check in the Require Authorization column. Details of the order can be viewed by clicking on the order (See FIG. 43). To authorize an order, click the check box to the left of any order. A prompt to authorize the order will be shown. To authorize the order, click Authorize (See FIG. 44). Click OK to proceed with the authorization. Before clicking OK, the Authorize check box from any order not to be authorized can be cleared. Note—changes to orders can be made before or after authorization of the order using the Order Entry screen (See FIG. 45).

The Order Entry screen allows the addition and of new orders to the patient's order profile and the modification of existing orders on the patient's order profile. Order Entry can be accessed from the Order Profile by clicking Go to Order Entry. To access Order Entry from any screen, once a patient is selected, click Order Entry in the menu options listed on the left side of the screen under Patient Options. The left side of the screen is used to search for orders. This is initially blank until a search is begun. From this side of the screen, orders can be searched, selected, and added to the patient's order profile. The right side of the screen displays active orders in the patient's order profile. It also displays new orders that have been added, but have not yet been saved to the patient's order profile. From this side of the screen, new orders that have not yet been saved to the patient's order profile can be removed or modified.

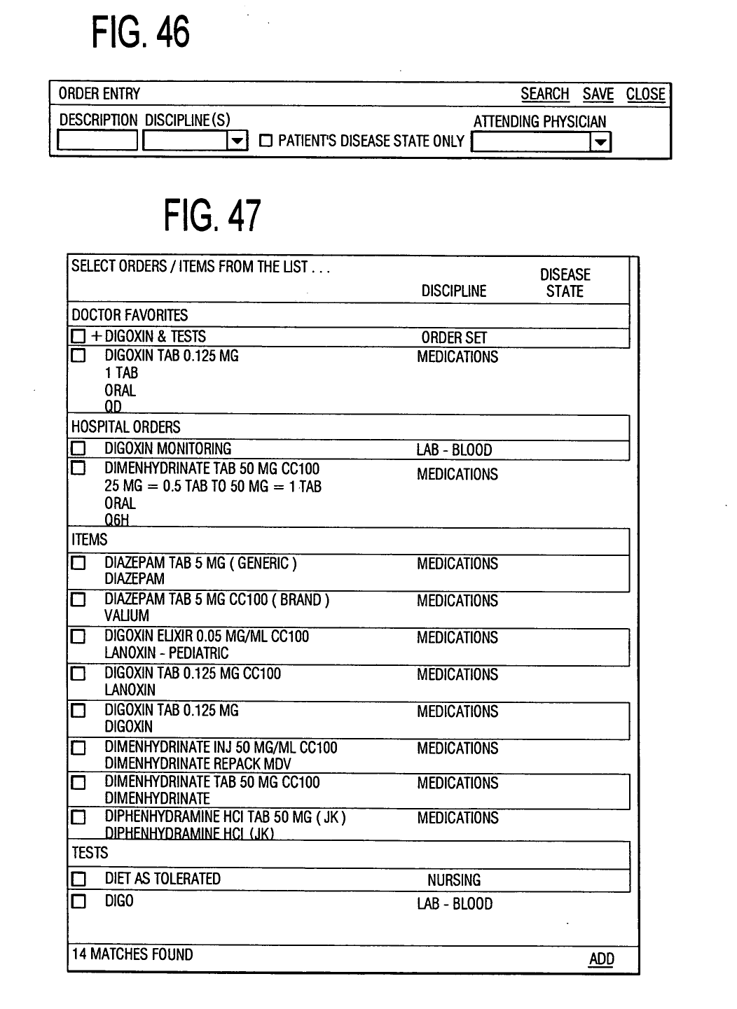

To search for items or orders to add to the patient's order profile, enter the appropriate search criteria at the top of the Order Entry screen, then click Search. The search results will be displayed on the screen (See FIG. 46).

Description indicates the order description, such as the item name, test name, or the name of an order set.

Discipline is used to restrict the search to one or more disciplines from the pre-defined list.

Patient's Disease State Only is a check box that allows a search to be restricted to only Orders or Order Sets that are specified for any one of the patient's current Disease States. Note—only Orders and Order Sets can have a disease state attached to them, so those are the only results when a search is restricted to Patient's Disease State Only. The results will not include orders or order sets that are not specific to a disease state.

Attending Physician allows the selection of another physician for searching for favorite orders and order sets of that physician. If no attending physician is selected, the user's favorites are displayed, as well as hospital-wide selections. Select a physician to search for favorites for that physician, as well as hospital-wide selections. This may be useful when placing an order for another physician. More than one search criteria can be used to narrow the search, e.g., Medications (underDiscipline) that start with dig (underDescription). The results of the search are displayed on the left side of the screen.

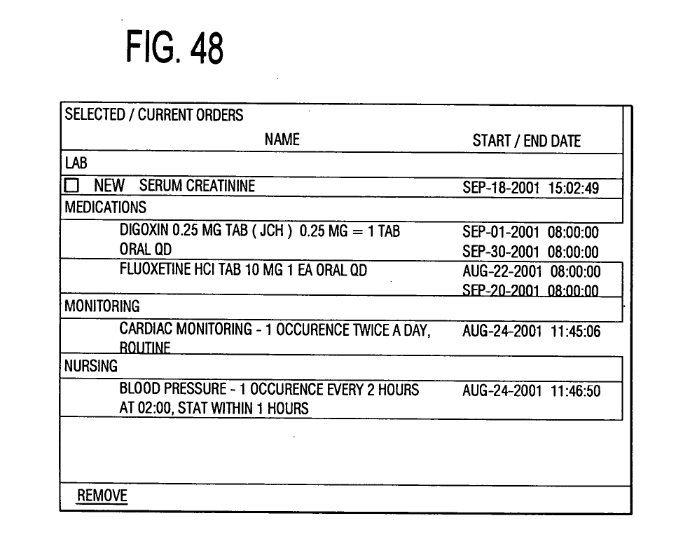

To add orders to the patient's order profile, select one or more orders/items, and click Add. The selections are added to the right side of the screen. Clicking Save will add the selections to the patient's order profile. The list on the left contains Doctor's Favorites, Hospital Orders, Items and Tests (See FIG. 47). The list can be sorted by Discipline or Disease State by clicking on the appropriate column heading. To select an order/item, click the check box to the left of it. Multiple selections may be made at one time from the list on the left. Order sets are listed as Order Set in the Discipline column, and have a “+” before them. To expand an order set, click the “+.” All the components of the order set are listed beneath the order set. An entire order set or individual components can be added to the patient's profile. By clicking Add on the bottom of the screen, all orders/items with check marks next to them are transferred to the right side of the screen. Before saving these changes, they are listed on the right side with a check box and a New indicator (See FIG. 48).

When an order set is transferred to a patient's profile, all the individual components of the order set (such as medications and tests) are transferred to the patient's profile. A listing of the individual components can be viewed on the right side of the screen. Before saving the changes, new orders/items can be removed from the screen by clicking the check box next to any order/item that should be removed from the right side of the screen, and clicking Remove on the bottom of the screen.

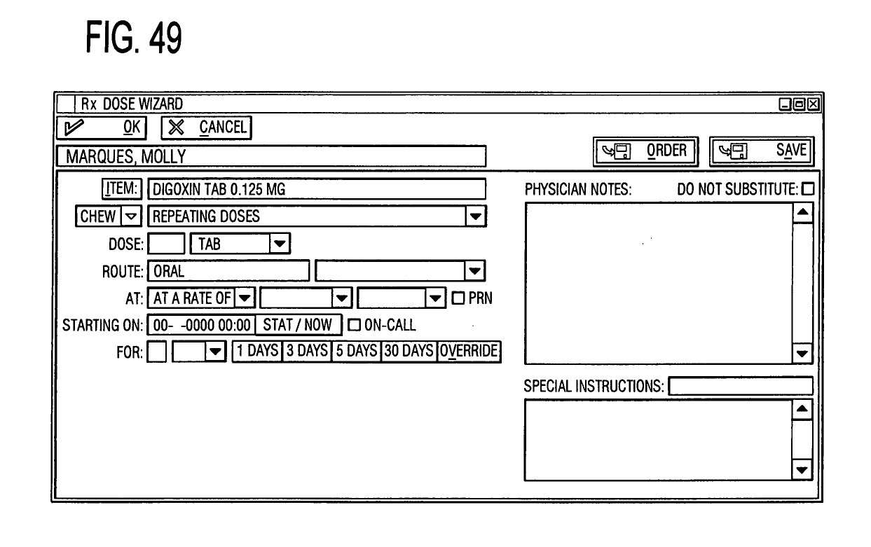

When medications that are Doctor Favorites or Hospital Orders are added, the selected standard order is transferred to the right side of the screen. When an item that is not part of a standard order, e.g., under the Items banner, is selected, a Rx Dose Utility or Wizard opens and prompts for information (See FIG. 49). After inputting the information, click OK, and the order will be added to the Selected/Current Orders list on the right with a New indicator. As used herein, a wizard is a software utility within a software application that helps a user use the application to perform a particular task.

When non-medication orders that are Doctor Favorites or Hospital

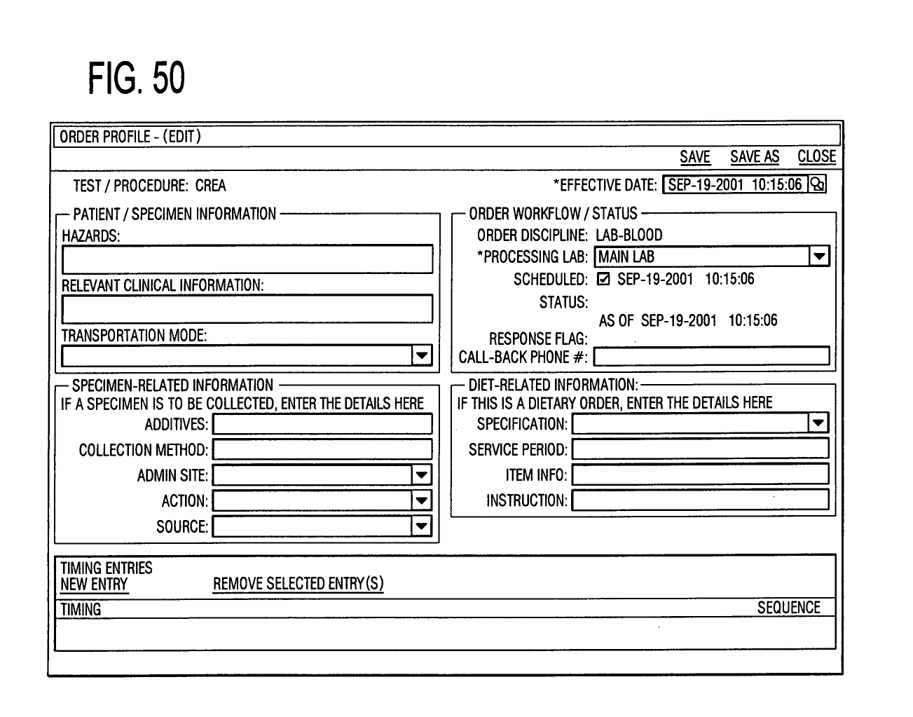

Orders are added, e.g., tests, the selected standard order is transferred to the right side of the screen. When a test that is not part of a standard order, e.g., under the Items banner, is added, the Order Profile (Edit) window will open (See FIG. 50). After inputting information into this window, click Save and the order will be added to the Selected/Current Orders list on the right with a New indicator. Note—this order can be saved as a Doctor Favorite by clicking Save As before Save. This will save the order as an Order Template and will be a Doctor Favorite.

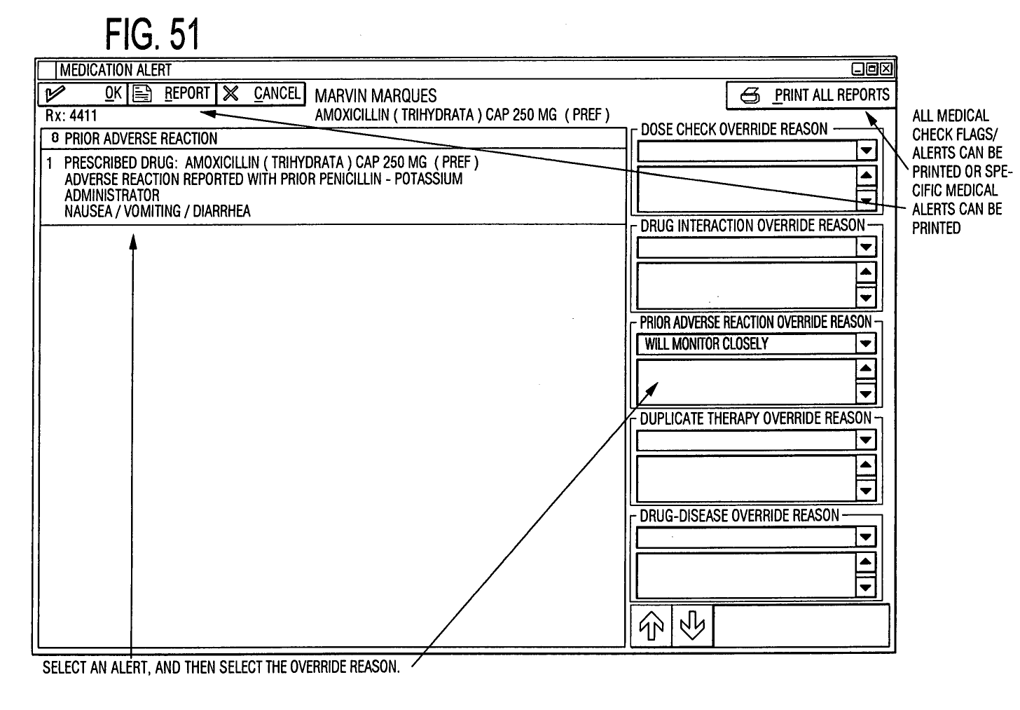

To save orders to the patient's order profile, click Save from the Selected/Current Orders List on the right side of the Order Entry screen. New orders will be saved to the order profile as will modifications to orders. At this point, the orders will be screened for irregularities. If any irregularities are detected with any prescribed drug, a Medication Alert screen automatically appears (See FIG. 51). All Medical Check Flags/Alerts can be printed or specific Medical Alerts can be printed. An alert shows one or more of the following discrepancies:

Prior Adverse Reaction indicates that a patient has an allergy noted on their profile for either this item or a derivative of the item.

Dose Checking indicates that the dose ordered is either above or below the recommended daily usage.

Duplicate Therapy indicates that the patient has a current active order for an item within the same therapeutic/subclass as the one currently being ordered.

Drug-Disease Contraindication indicates that the drug ordered is contraindicated for a disease state noted on the patient's profile. For example, if pregnancy is a disease condition state and Tetracycline is ordered, a Medication Alert window would appear.

Drug Interaction indicates that the drug ordered is being co-administered with one or more other drugs, and that this combination produces unwanted or unexpected pharmacologic reactions.

Medical Alerts can also be overridden by selecting the alert and then providing an override reason. On the Medication Alert window, an override reason can be provided or selected from a predefined drop down list. Once the reason is selected, click OK to continue. The order will not appear on the patient's order profile until the Medication Alert window is completed.

Once the orders are saved to the patient's order profile, the Order Profile screen is displayed. To modify an order, click on the order name on the Order Entry screen from the Selected/Current Orders list on the right side. A window will open from which changes can be made. A window will open depending on whether the order is new (indicated by the New flag) or active, and whether or not the order is for medication. To save any changes, click Save in the Order Entry screen. Orders can also be saved to the Order Profile.

The Rx Dose Utility opens when a new medication order is modified. Any necessary order modification are saved by clicking OK and will appear on the patient's order profile. Modification of a new non-medication order requires opening the Order Profile (Edit) window. Any necessary modifications to the order can be made and saved by clicking Save. The saved changes appear on the patient's order profile.

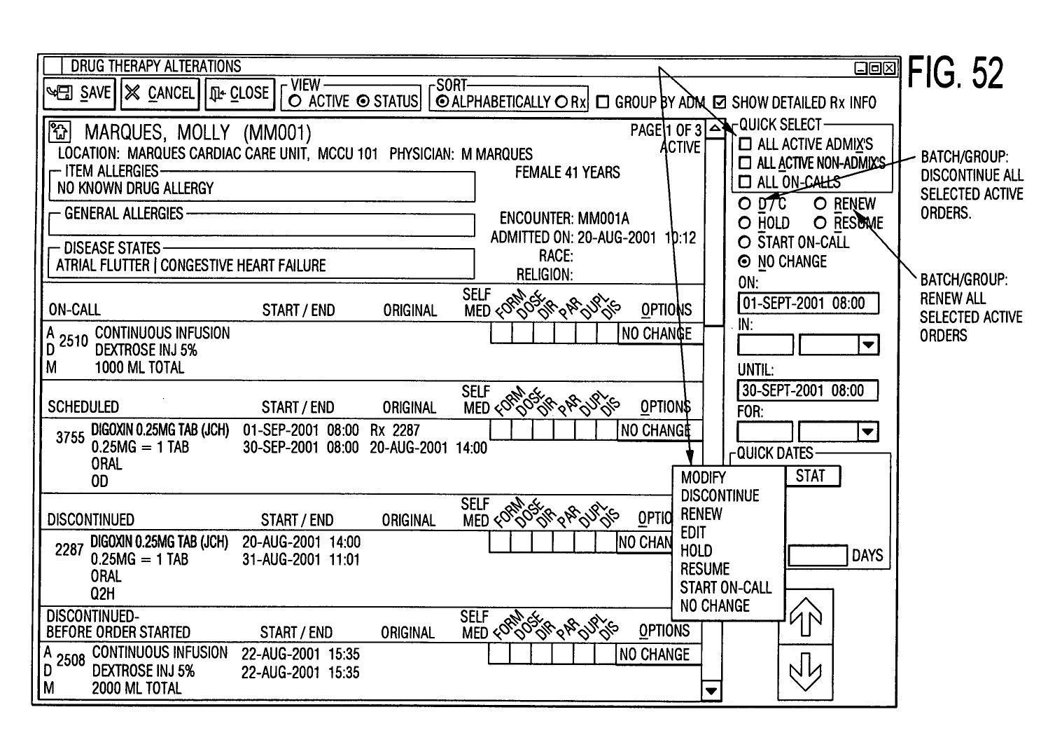

Changes to an active medication order requires opening the Drug Therapy Alteration window (See FIG. 52). From this window, modifications to any medication order can be made and saved. All orders that are modified or resumed, as well as all inactive orders that will become active, are screened for irregularities. A proper response may be required for any medication alerts. Note—only active (including on-hold) and new orders are listed in the Order Entry window. To renew an expired order or to start an on-call order, click any active medication order. The Drug Therapy Alteration window also lists expired, on-call, on-hold, and discontinued orders. Changes to the order can be made from this window.

To modify an individual order, activate the No Change button beside the order and select the required type of change from the pop-up list, e.g., Modify or Discontinue. By clicking on a specific row's option column and selecting the action desired, the changes only affect that individual order. Conversely, by using the group options on the right hand side of the window, the changes affect all the highlighted rows. A Batch/Group selection can be made to discontinue or renew all selected active orders.

If Modify or Edit is selected, the Rx Dose Utility will open and the required changes can be made. The date(s) list should be adjusted, if necessary. For some types of changes, e.g., Discontinue or Hold, the date will be listed below the button. For all modifications, the starting On and/or Until dates are displayed on the right.

Similar changes required for a number of orders, e.g., discontinue/renew all active orders, can quickly be made by selecting all the orders at once. The Quick Select area allows swift selection of All Active Admixtures, All Active Non-Admixtures, or All On-Calls. Specific selection of orders from a list is also capable. To select a range of orders, click on the first order, then shift-click on the last order. To select non-adjacent orders or to clear specific orders from a selection, control-click on the order. Note—discontinued or expired orders cannot be placed in the group selection and must be modified individually. Once an appropriate action is selected, e.g, Discontinue, Renew, Hold, Resume, Start On-Call, No Change, the date(s) listed can be verified and adjusted as necessary. The starting On and/or Until dates are displayed on the right. Date changes can be made either directly in the field, through the In or For selections to indicate a date and time relative to the current date and time, or through Quick Dates. Changes are saved by clicking Save.

Various alterations can be made from the Drug Therapy Alteration window. For example, Modify allows the modification of active, discontinued or expired orders. Selecting Modify brings up the Rx Dose Utility allowing for desired changes to be made. If the order is an active order, the old order will be discontinued and a new order will be started with the specifications indicated in the Rx Dose Utility. Note—the duration of the order is from the current date and not from the original start date of the order. Once an order has been modified and before it is saved, further changes can be made by accessing the Edit function to get back to the Rx Dose Utility.

Selecting Discontinue allows for the discontinuation of any active order. By default, the order is discontinued immediately, but may also be scheduled to be discontinued at a future time. If the ordering physician is other than the person signed-on, the user may select the ordering physician's name by left then right clicking on the authorized by box and highlighting the name. When a continuous infusion is discontinued and the discontinue time is at the scheduled administration time or within the time that the administration could be given (without being early or late), a decision can be made as to whether or not that particular administration should be given. When a continuous infusion order is discontinued, a clinician will receive a message providing a time to take the infusion bag down.

An active or expired order can be renewed by selection of the Renew option. The default renewal time is determined by each facility, but may be altered as required.

Any active order may be placed on hold by utilizing the Hold option. The order is not discontinued when it is placed on hold and it can later be resumed. The order will expire if it is not resumed before the end date of the order. If a continuous infusion order is placed on hold, a clinician will receive a message instructing the time to take the infusion bag down.

To resume an order, select the Resume option. When a continuous infusion order is resumed, a message instructing when to put the infusion bag back up will be sent to the clinician.

It is possible to exclude certain drug orders of a selected group action from the right side of the Drug Therapy Alterations screen, e.g., Renew All Active, from being changed. The No Change function is used in such a case. Before saving, click on the button that is currently showing one of the four other choices of a modified order, e.g., D/C, Hold, Renew or Resume, in the Options column of the Drug Therapy Alterations screen and change the option button to No Change. This will ensure that no change will occur to the selected drug order(s).

Any on-call order can be started by selecting the Start On-Call option.

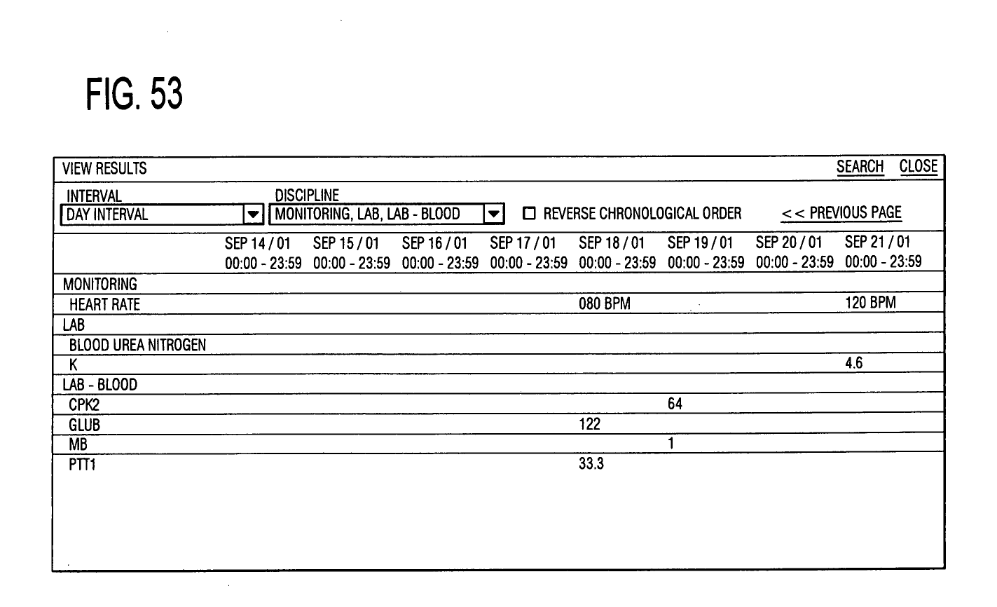

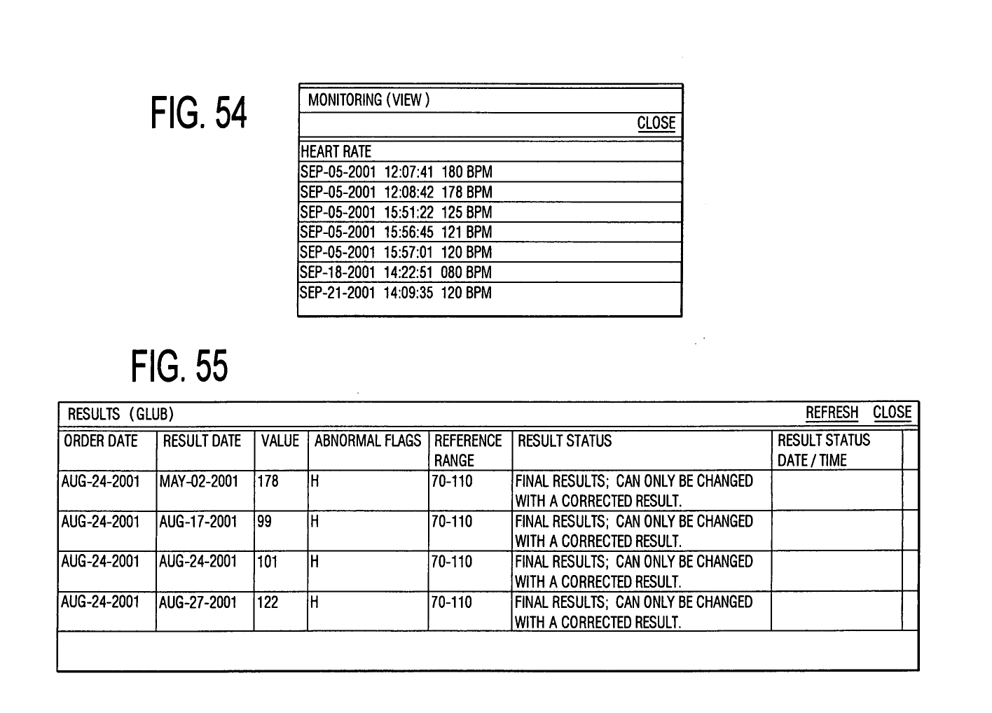

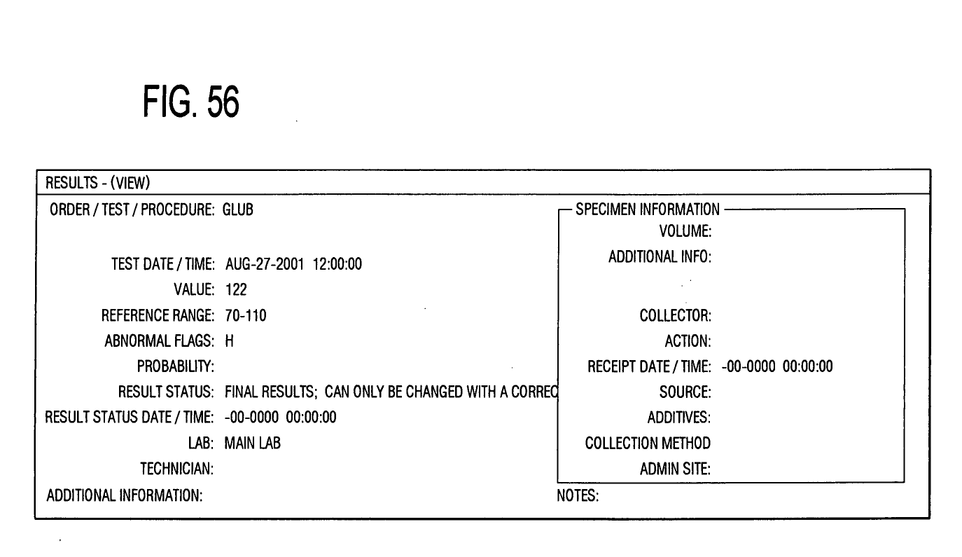

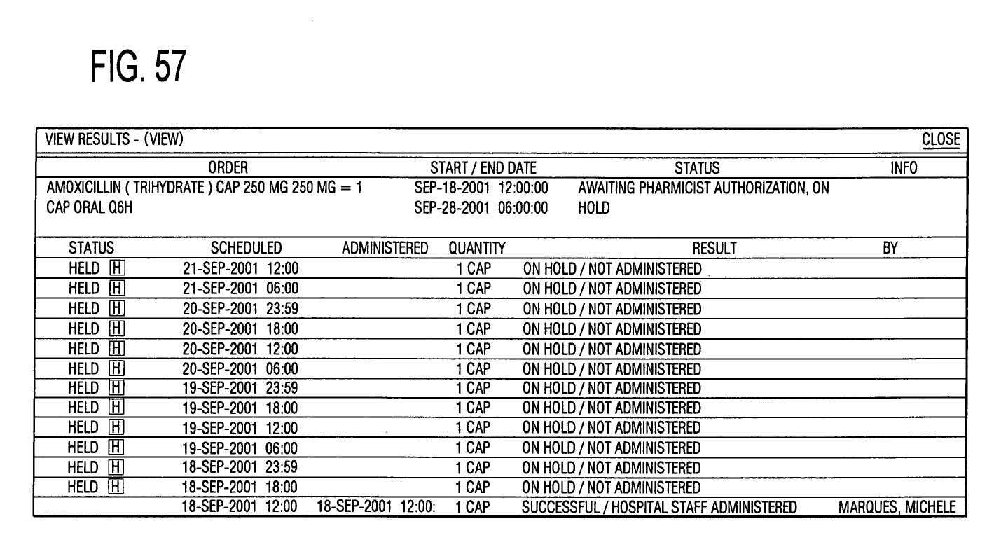

Information related to laboratory tests or administered medications can be viewed in an organized fashion across multiple disciplines (See FIG. 53). The view and interval of the displayed results will be saved until changed. By default, the results are displayed in chronological order. The results can also be viewed in reverse chronological order by selecting the Reverse Chronological Order option. The results can be viewed in a graphical format. Clicking Search while viewing the screen will update the results and additional results will be displayed if they have recently occurred. Clicking the description of any result will provide a detailed summary of the selected results. The detailed summary varies depending on the type of result. FIG. 54 is an example of monitoring results. FIGS. 55 and 56 are examples of test results. FIG. 57 is an example of medication results. For results that occur infrequently, e.g., hemoglobin, the look-back period can be changed to include all results desired so that instead of scrolling day-by-day through results, the detailed summary will list all results, but will not include time periods for which there are no results.

It is to be understood that the present invention can be integrated with additional resources that may be available at certain facilities. Various external applications may be launched from within the application. For example, users may be provided with other applicable clinical applications or reference materials.

Many configurations utilize AUTROS Inpatient and AUTROS Maintenance applications by Pharmacy and Database Administrator(s). These applications provide options that allow configuration of Order Templates and Order Set Templates. These templates are available during order entry as Doctor Favorites.

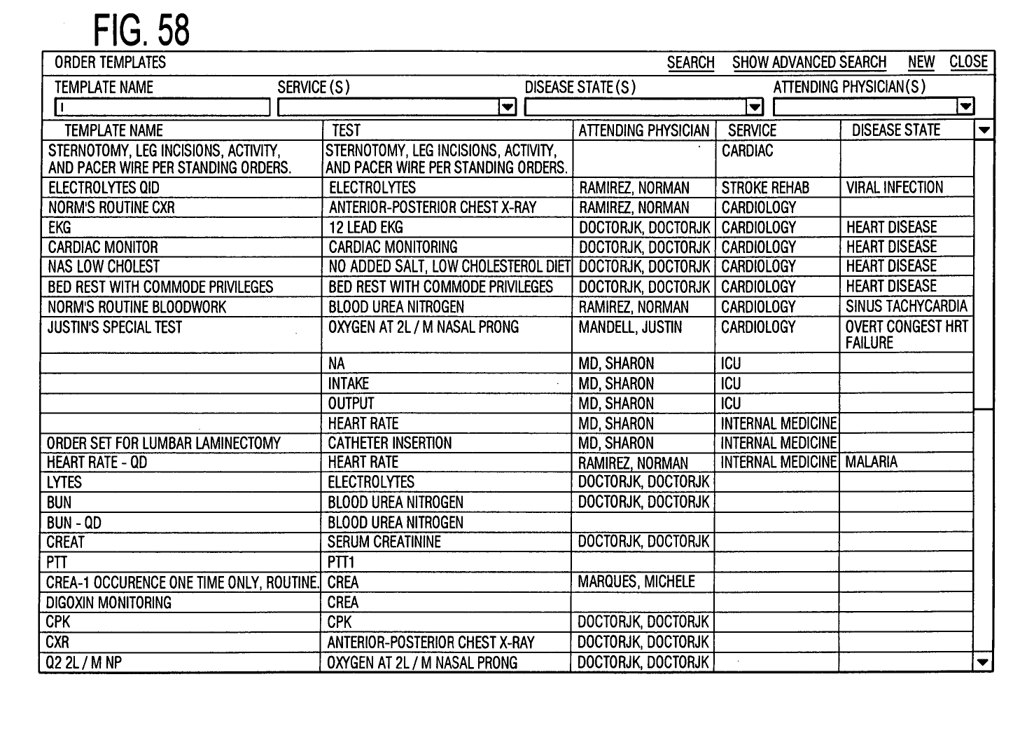

Order Templates allow saving of individual non-medication orders, e.g., tests, that are frequently ordered and are available either as Doctor Favorites or Hospital Orders during order entry. Order Templates are also used to build Order Set Templates that are used to save groups of orders for easy ordering. Note—to save individual medication orders to be available as Doctor Favorites, begin by placing an order for the item, then, from the Rx Dose Utility, click Save. By clicking Order Templates, the Order Templates screen is displayed listing all Order Templates in the system (See FIG. 58).

Several techniques are available for locating a specific order template. The search results can be filtered to match the desired criteria, or the results can be sorted by column. At the top of the screen is an area for entering the search criteria. The results can be filtered by Template Name, Service(s), Disease State(s), or and/or Attending Physician(s). If the filter is a list, multiple selections may be utilized. For less frequently used filters, e.g., Disease State(s), click Show Advanced Search and an additional filter will be available for filtering on part of the Disease State Name. After entering the search criteria and clicking Search, the filtered results are displayed. Each added search criteria further restricts the results. For example, if a search includes a particular physician and disease state, the results will only list that physician's templates for the selected disease state. To sort the results by a column, click the heading of that column.

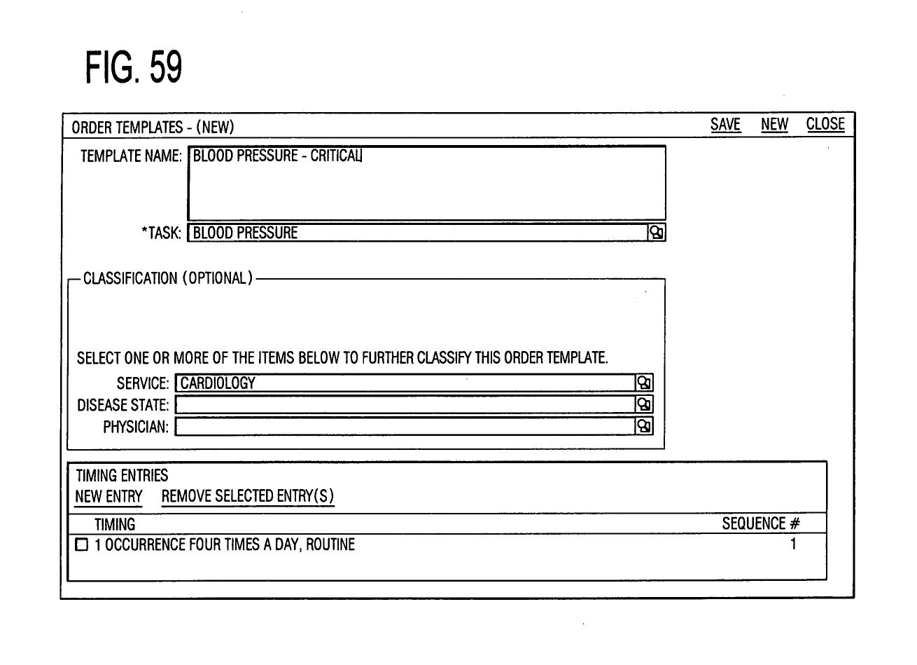

To create a new order template, click New and the Order Templates—(New) screen is displayed (See FIG. 59). Within this screen, Template Name is used to identify a preferred non-drug order during order entry. Although the Template Name is optional, it is recommend that a value be entered once this order template is ready to be used. The Test field must specify a test or other non-drug order. Searching for a test is initiated by clicking the search icon.

The Service field is optional and can be utilized to search for Order Templates or to sort them when maintaining Order Templates, or when building Order Set Templates. During order entry, search orders can be restricted to a patient's disease state by utilizing the Disease State field. The Disease State may also be used to search for and sort Order Templates. If the Physician field is blank, the Order Template will be a Hospital Order; otherwise, it is a Doctor Favorite. Hospital orders are hospital-wide standards and are displayed during order entry regardless of the doctor. Doctor Favorites are specific to a physician and are displayed during order entry only if that physician is signed-in, or if the user selects that physician as the attending physician.

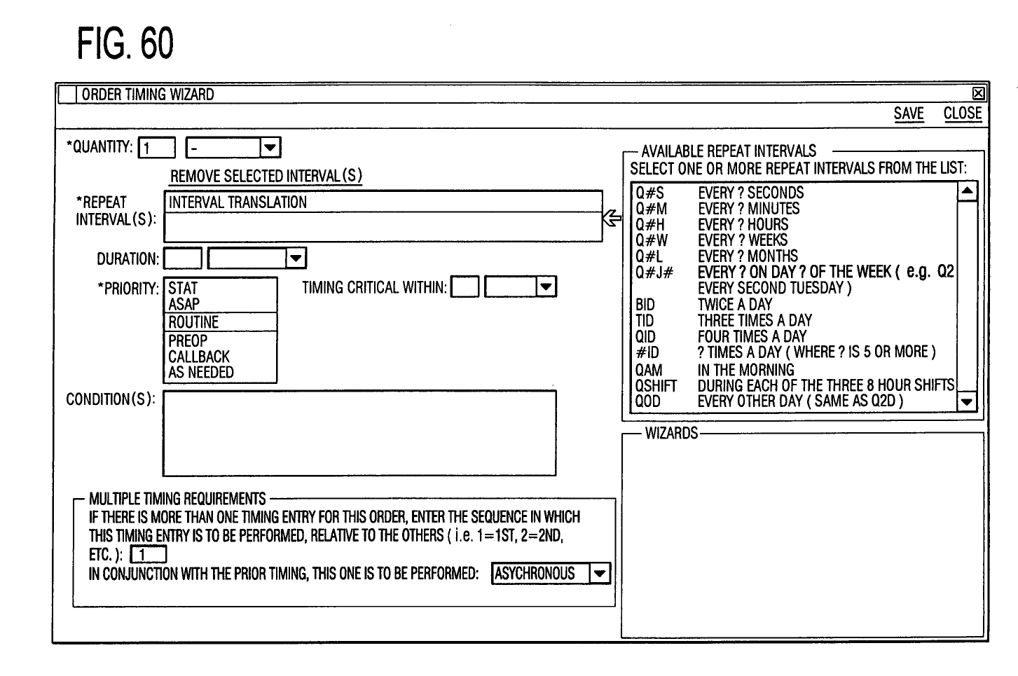

The performance timing or frequency of a test or procedure can be indicated by utilizing the Timing Entries field. Changes made to the Orders Template—(New) screen can be saved by clicking Save and Close to save and close the screen. When adding a new timing entry, the Order Timing Utility or Wizard appears (See FIG. 60). Various fields can be populated. Preferably, any field requiring information will be identified by an asterisk (*). The various fields that can be populated on the Order Timing Utility screen include: Quantity, Repeat Interval(s), Duration, Priority, Timing Critical within, Condition(s), and Multiple Timing Requirements.

The Quantity field is attached to the timing entry and has a default of 1 occurrence. When the measure is a time period, then occurrence is indicated in the timing entry. A different quantity and appropriate unit of measure can also be entered, e.g., volume and vial(s) or unit(s).

A repeat interval is selected from the Repeat Interval(s) list on the right. When a repeat interval is selected, the Utilitys area brings up time selectors. Optionally, specific time(s) for a test to be given or a procedure performed can be indicated The Repeat Interval determines when a test or procedure should be given. Timing Critical within indicates the latitude for the test or procedure occurring at a different time. A proper quantity and time unit should be placed in this field, e.g., 5 minutes, 2 days. The Duration of a test or procedure can also be indicated. The quantity can be specified as well as the measurement, e.g., seconds, hours, or dosage totals.

Various levels of Priority can also be selected. The default priority is Routine. If relevant, information about Condition(s) can also be entered.

If there are Multiple Timing Entries, this section must be completed. By default, the sequence numbers are the same order in which the Timing Entries were created. The sequence numbers must be unique and may be changed By default, the current timing entry is to be performed Synchronous to the previous entry (that is, after the previous entry is complete). This can be changed to be performed Asynchronous with the previous entry (i.e., the timing is independent of the previous entry).

Clicking on any Order Template will display the details. From this screen, changes to the Order Template can be made or the Order Template can be deleted. Order Set Templates allow a group of orders to be saved. This is beneficial for orders frequently ordered at the same time to be available as Doctor Favorites during order entry. Order Set Templates may include both medication and non-medication orders. During order entry, the entire set may be ordered at once, or individual orders may be selected from the order set. Before an Order Set Template can be created, the appropriate medication orders and non-medication order templates must be saved. When an Order Set Template is created, the appropriate orders and order templates will be grouped together.

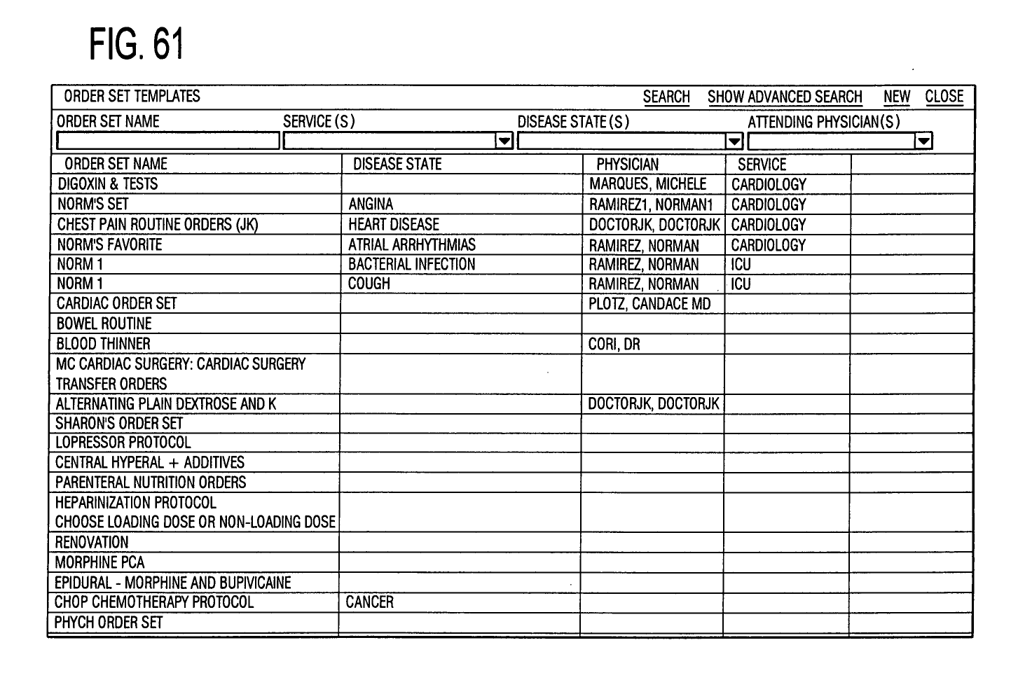

By clicking Order Set Templates, the Order Set Templates screen is displayed (See FIG. 61). This screen does not list Order Set Templates unless a search was performed. At the top of the screen is an area for entering the search criteria. A search may be made by Template Name, Service(s), Disease State(s), and/or Attending Physician(s). When the criteria is a list, multiple criteria can be selected. As discussed above, if use of a particular criteria is not desired, e.g., Disease State(s), click Show Advanced Search and an additional filter will be available for filtering on part of the Disease State Name. After entering the search criteria and clicking Search, the filtered results are displayed. Each added search criteria further restricts the results. For example, if a search includes a particular physician and disease state, the results will only list that physician's templates for the selected disease state. To sort the results by a column, click the heading of that column.

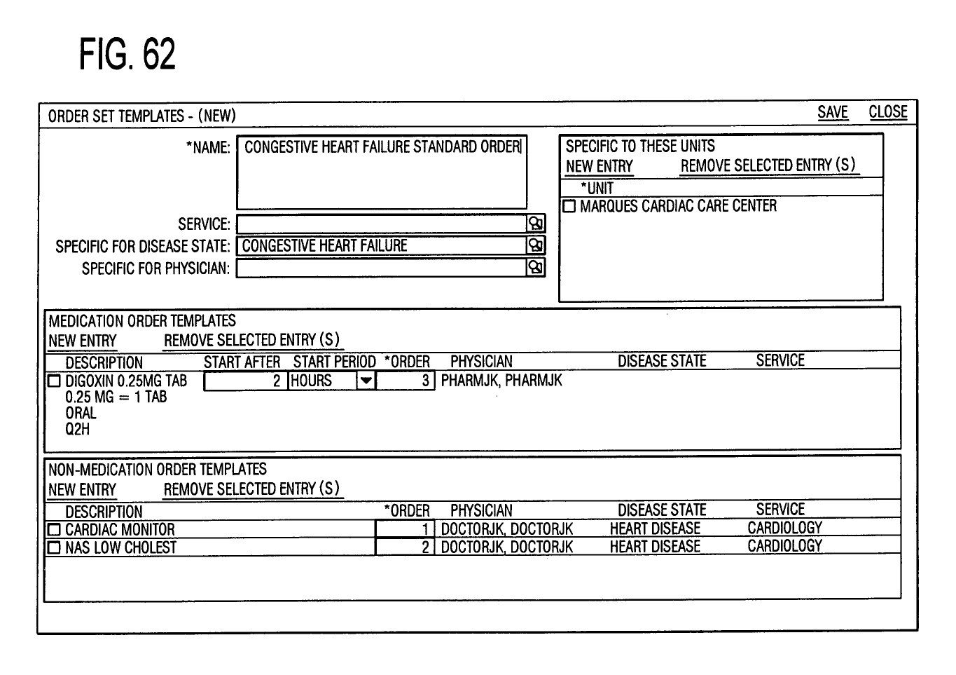

To create a new order template, click New. Order Set Templates—(New) to display the appropriate screen (See FIG. 62). Several fields are populated to complete the screen. One required field is Name and it is used to identify order sets during order entry. The Service field can be utilized to search for Order Set Templates or to sort them. Disease State can be utilized during order entry to restrict the search to orders specified for a patient's disease state. The disease state may also be used to search for and sort Order Set Templates.

The Physician field indicates whether the Order Set Template will be a Hospital Order or a Doctor Favorite. If this field is blank, the Order Set Template will be a Hospital Order; otherwise, it is a Doctor Favorite. Hospital orders are hospital-wide standards and are displayed during order entry regardless of the doctor. Doctor Favorites are specific to a physician and are displayed during order entry only if that physician is signed-in, or if the user selects that physician as the attending physician.

An order set can be selected that is specific to one or more units, i.e., Specific to These Units. Clicking on any Order Set Template will display the details on the screen. From this screen, the Order Set Template can be changed or deleted.

Both medication and non-medication orders can be attached to an order set template. At least one order must be attached. Orders must be saved before being attached to an Order Set Template. When an order is attached, the order in which it is to be performed must be specified. By default the Order is the order in which the orders were added. When a medication order is attached, the order to start relative to when the order is placed can be specified. The Order Set Template can be saved by clicking Save.

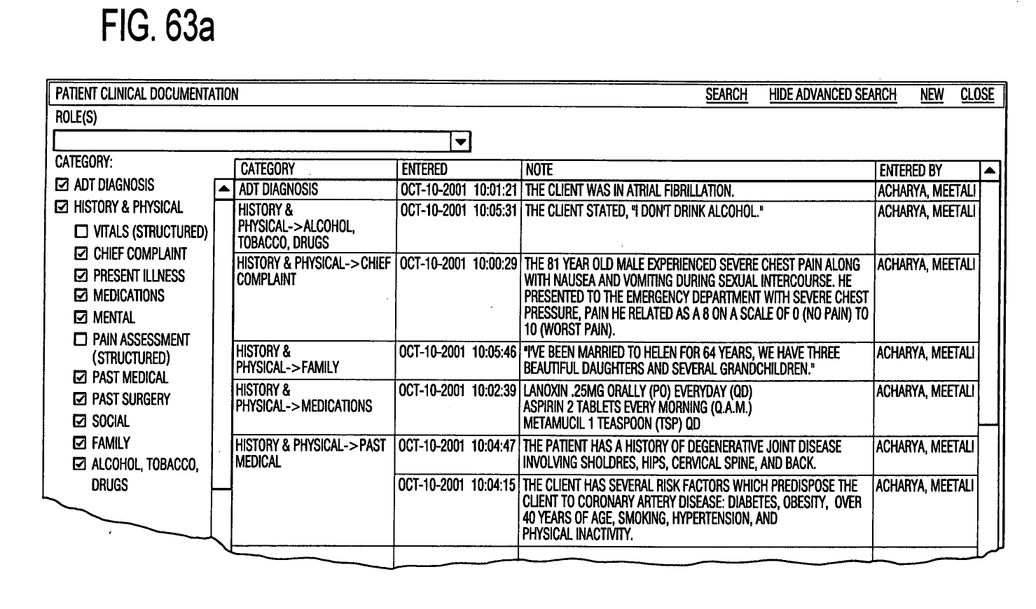

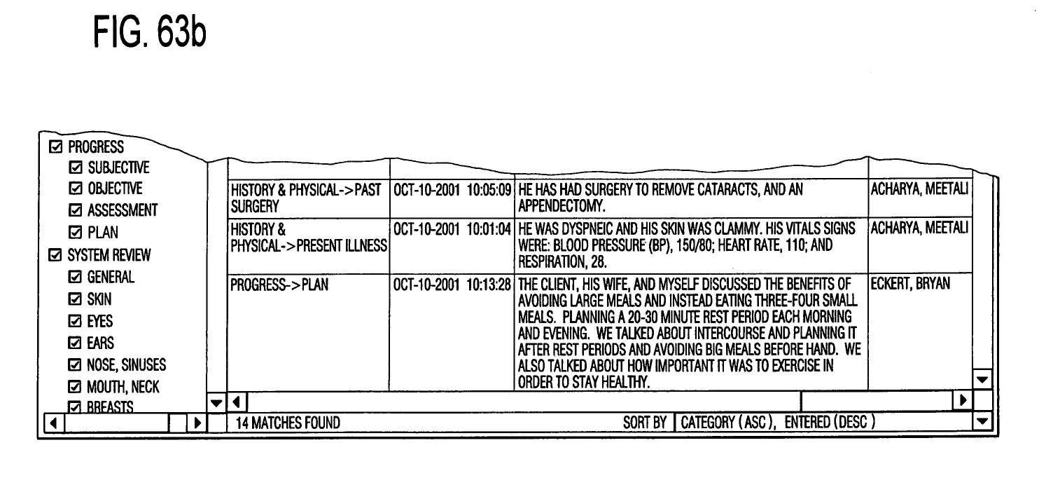

In general, the present invention facilitates patient care by providing an application where medical clinicians, e.g., physicians, nurses, respiratory therapists, physiotherapists, etc., can document patient-related notes relating to assessments, progress, and plans. The application utilizes forms to provide the clinical documentation in a structured format for easy access to record, view and retrieve the information. Several categories exist within clinical documentation that are capable of being maintained by the system. These categories include, but are not limited to: admitting diagnosis, history, physical, progress, and notes. These categories can be modified and additional categories and sub-categories can be added. The display of these categories can be viewed using a Patient Clinical Documentation window (See FIG. 63).

When a new clinical documentation note is entered, various pieces of information can be recorded under specific headings. These include: category, subcategory, Date/Time the note was entered, the actual note, encounter locator, whether that note requires authorization (verification), who entered the note, the authored date, the author role, and the date it was authorized. The information can be sorted using the above headers.

Clinical Documentation notes can be searched according to the Role of the person that entered the note into the system, e.g., nurse, physician. The search is in the form of a multi-select drop down. Searches can also be performed using the categories and subcategories. By checking the appropriate box, the system retrieves only the notes that were entered under the selected categories and/or subcategories.

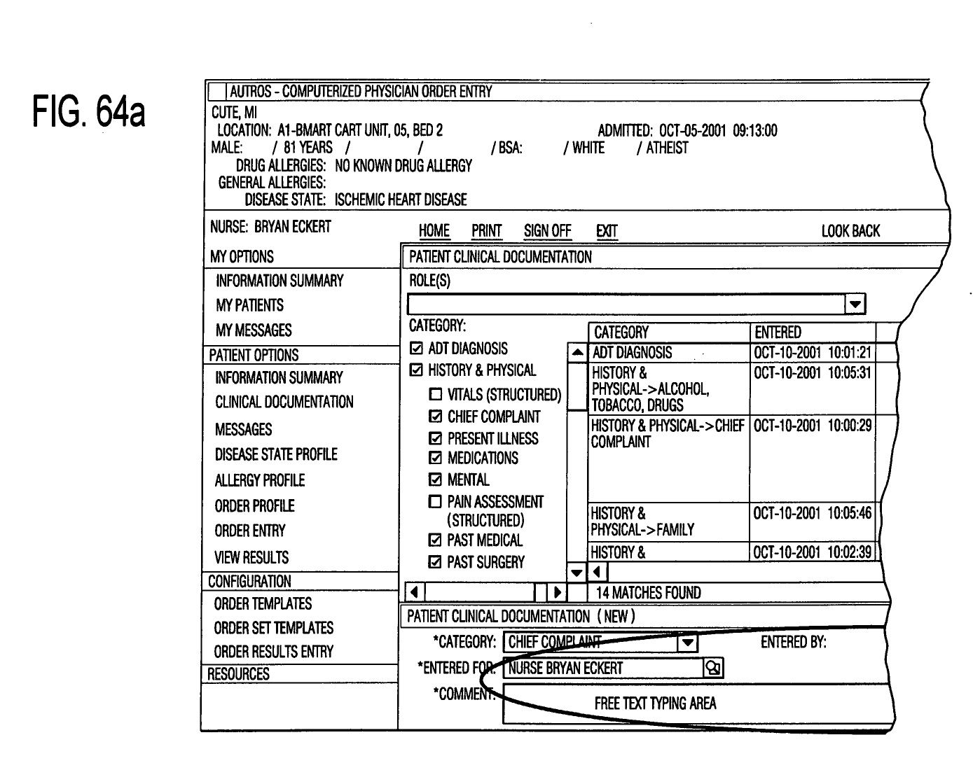

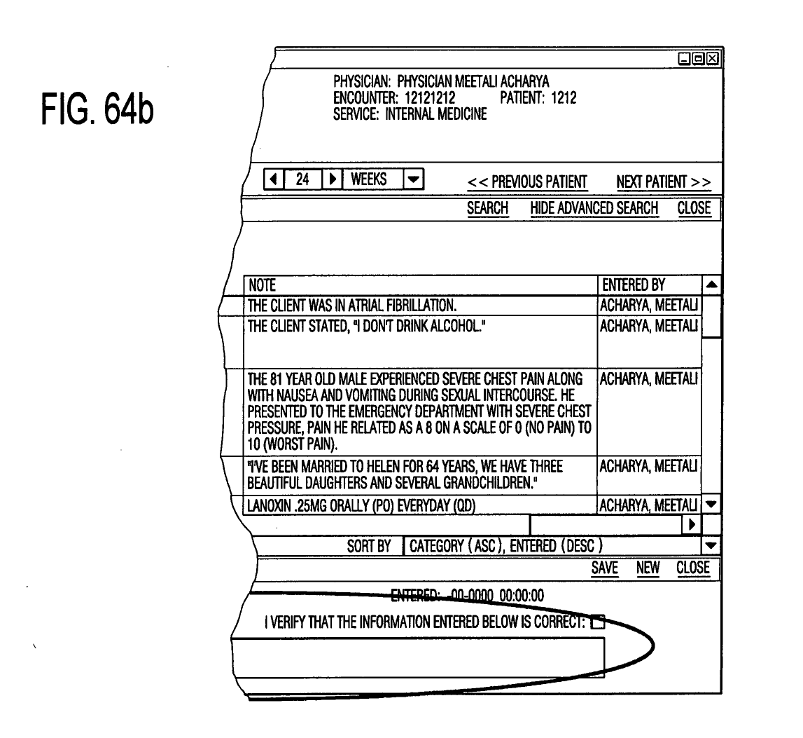

Effectively, the user selects the category for attaching a note. By clicking on an existing note, the bottom half of the screen expands to display more detailed information and also any previous or subsequent documentation related to the entry. If a clinical documentation note has been entered on behalf of the clinician, the clinician has the ability to sign off on the note electronically through the authorization feature in clinical documentation (See FIG. 64).

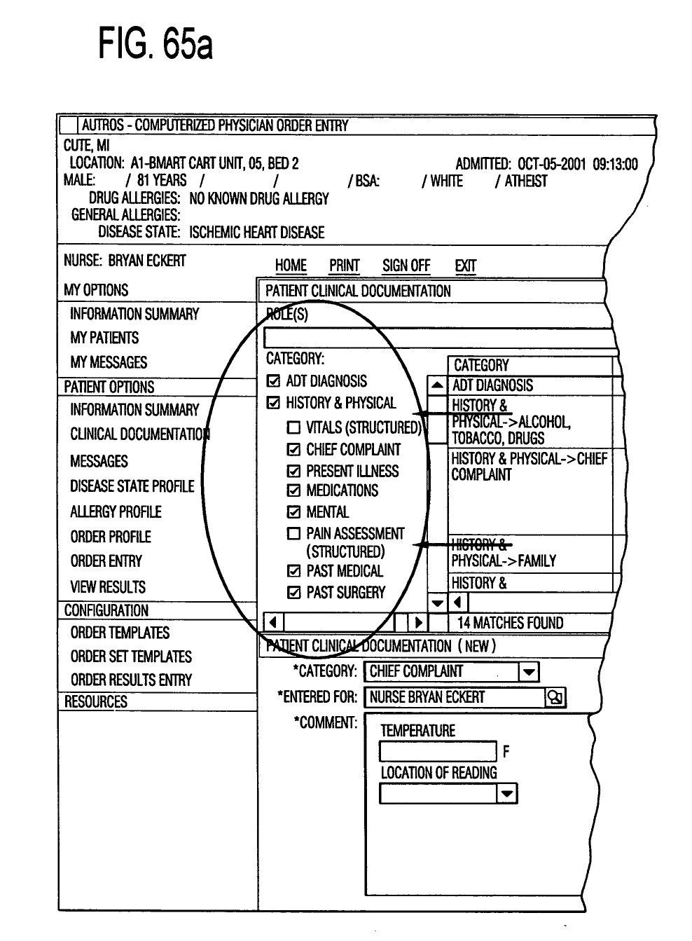

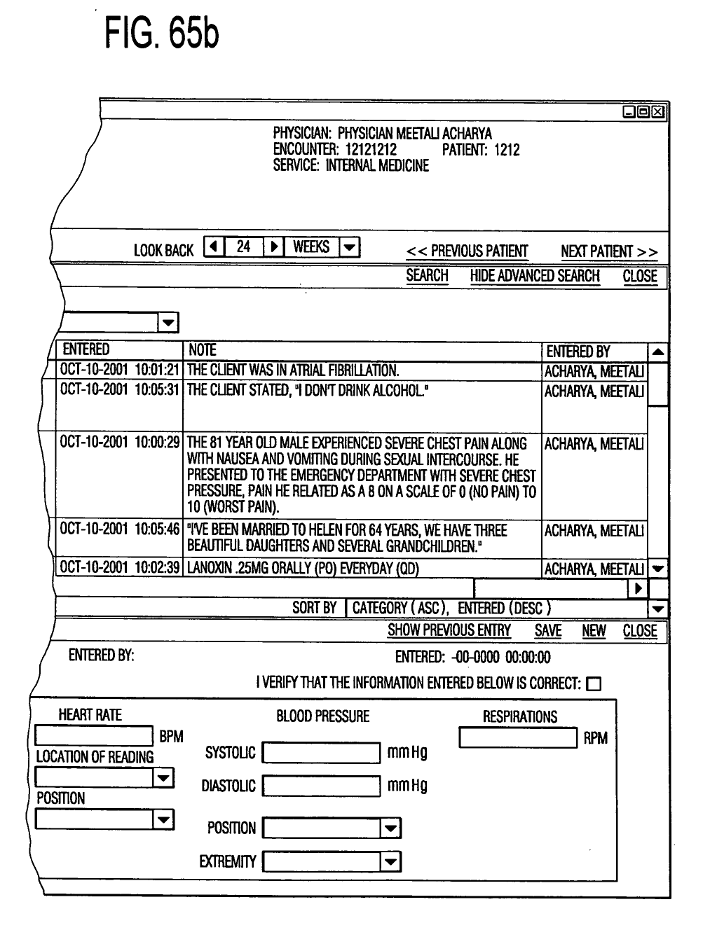

Structured documentation is displayed in the clinical documentation window as a string of information and/or as part of results viewing. The structured documentation utilizes the existing functionality within monitoring parameters. Although monitoring parameters results differ from clinical documentation, it is often necessary to view a patient's total results—both noted documentations and monitoring parameter/lab/diagnostic result reporting together in chronological or reverse chronological order (See FIG. 65). Therefore, it is preferable that users be allowed to view all results for a patient from the clinical documentation screen. When results, other than clinical documentation notes, are viewed from the clinical documentation screen, the format of display is a text blob in which the data is separated by a pipe, |. The information is not stored in clinical documentation, but is instead retrieved for viewing purposes only.

In the maintenance application, when creating clinical documentation categories and sub-categories, the user has the ability to retrieve result name(s) from any discipline and to attach more than one result name to a category definition. The audit trail/history on the creation of these category definitions are maintained by the system. It is preferable to allow the user to determine the types of results from the results window that are to be viewed in the clinical documentation window. This is applicable to any discipline's results and not be restricted to monitoring parameters.

A user is also able to enter new structured results within Clinical Documentation. Using monitoring parameters as the basis, the application provide the ability to record new structured notes within Clinical Documentation. When the user clicks on the “New” hyperlink on the top right side of the screen, the bottom half of the screen explodes such that a new note can be recorded. If the category that is chosen for recording the note is a structured (result-oriented) category, the blank monitoring parameter associated with that category/subcategory is displayed and the user is allowed to fill in the information required on the form. If more than one monitoring parameter is attached to that result-oriented category, a pertinent list of monitoring parameters is displayed so that the user can choose the monitoring parameter on for which they wish to record information. Once a monitoring parameter is chosen, the blank monitoring parameter is displayed and the user can fill in the information required.

It should be emphasized that the above-described embodiments of the present invention, particularly, any “preferred” embodiments, are possible examples of implementations, merely set forth for a clear understanding of the principles of the invention. Many variations and modifications may be made to the above-described embodiment(s) of the invention without substantially departing from the spirit and principles of the invention. All such modifications are intended to be included herein within the scope of this disclosure and the present invention and protected by the following claims.

What is claimed is:

1. A system for facilitating documentation preparation utilizing preexisting material to improve treatment of the patient, the system comprising:
a processor;
a configuration module;
a memory operably connected to the processor, and,
a record of encounters being stored in the memory, wherein the configuration module, processor, and memory cooperate to generate an order utilizing the record.

2. A system for clinical documentation, comprising:
a processor; and,
a configuration module cooperating with the processor and adapted to allow an authorized user to select existing encounters, the user being able to copy a portion of the existing encounters into a new encounter.

3. For a clinical documentation system, a method for facilitating creation of clinical documents comprising the steps of:
providing for receiving a request to copy at least a portion of an existing clinical document;
providing for sending an existing clinical document to copy in response to receiving the request;
providing an electronic system for copying at least a portion of the existing clinical document; and,
providing for copying at least a portion of the existing clinical document into a new clinical document.

4. The method for facilitating creation of clinical documents of claim 3, further comprising the step of:
providing for selecting the portion of the existing clinical document to be copied.

5. The method for facilitating creation of clinical documents of claim 3, further comprising the steps of:
providing for determining an access status of the user, and if the user has appropriate access privileges, then providing for copying a portion of the existing clinical document into the new clinical document.

6. The method for facilitating creation of clinical documents of claim 5, further comprising the step of:
providing for determining access based on individual portions of the clinical document.

7. The method for facilitating creation of clinical documents of claim 3, further comprising the step of:
providing for allowing the user to edit the new clinical document after the copied material has been placed in the new document.

8. The method for facilitating creation of clinical documents of claim 3, further comprising the step of:
providing for validating the new clinical document.

9. The method for facilitating creation of clinical documents of claim 3, further comprising the step of:
providing for producing an encounter order as the clinical document.

10. The method for facilitating creation of clinical documents of claim 3, further comprising the step of:
providing for tracking modifications made to the clinical document.

11. The method for facilitating creation of clinical documents of claim 3, wherein the tracking further comprises the steps of:
providing for tracking information related to the date of copying;
providing for tracking an identification of the document from which the copying was performed; and,
providing for tracking an identification of the document into which the copied material was directed.

12. The method for facilitating creation of clinical documents of claim 3, wherein the method for copying an existing order comprises:
providing for selecting the existing clinical document;
providing for copying at least a portion of the existing clinical document into memory; and,
providing for pasting the at least a portion of the existing clinical document into the new clinical document.

13. The method for facilitating creation of clinical documents of claim 3, wherein the clinical document is an order.

14. The method for facilitating creation of clinical documents of claim 3, further comprising the step of:
providing for automatically copying the selected portion of the document into the new document when only one active new document exists.

15. A computer readable medium for a system to facilitate treatment of a patient, the system includes a processor and a memory for maintaining a record of encounters between the patient and a health care facility, the medium comprising:
a first code segment for determining access status of a user;
a second code segment for providing a means for copying an existing order
a third code segment for receiving a request to copy an existing order;
a fourth code segment for determining the copy status of the existing order;
a fifth code segment for copying the existing order into a new order;
a sixth code segment for editing the new order; and,
a seventh code segment for validating the new order.

16. A system for providing guidance during treatment of a patient, the system comprising:
an event comprising at least one patient characteristic;
a processor;
a memory being operably connected to the processor, the memory being capable of storing patient records, the patient records comprising patient characteristics;
a search module, the search module being capable searching the memory for a patient record matching the event; and,
an alert being activated in response to the search module.

17. The system of claim 16, wherein the search module is an end-user programmable application.

18. The system of claim 16, wherein the alert is a text based message.

19. The system of claim 16, wherein the alert includes clinical guidelines or suggestions.

20. The system of claim 16, wherein the alert comprises a change in patient therapy.

21. The system of claim 20, wherein the change in patient therapy is automatic.

22. The system of claim 20, wherein the change in patient therapy is completed only after clinician authorization.

23. The system of claim 16, wherein the alert comprises recommendations to a patient therapy.

24. The system of claim 16, wherein the alert comprises a recommendation for a laboratory test.

25. The system of claim 16, wherein the alert comprises a recommendation for clinical monitoring.

26. The system of claim 16, wherein the alert comprises an indication of inappropriate medication dosing for a particular age patient.

27. The system of claim 16, wherein the alert comprises an indication of suggested medications for a specific patient based on the patient characteristics.

28. The system of claim 16, wherein the alert comprises an indication of suggested tests for a specific patient based on the patient characteristics.

29. The system of claim 16, wherein the patient records comprise defined categories.

30. The system of claim 29, further comprising an audit of changes being conducted to the patient records.

31. A system for providing guidance during treatment of a patient, the system comprising:
an event entered into the system by a user;
a processor;
a memory being operably connected to the processor, the memory being capable of storing records;
a search module, the search module being capable searching the memory for a record matching the event; and,
an alert being activated in response to the search module.

32. The system of claim 31, wherein the event comprises at least one patient characteristic.

33. The system of claim 31, wherein the event is at least one of gender, age, race, allergies, past medical history, present illness, risk factor for a particular disease, laboratory results, diagnostic results, vitals, medication ordering or medication administrations.

34. For a system including a memory of patient information, a method for providing decision support to healthcare personnel during treatment of a patient, the method comprising the steps of:
providing for defining an event;
providing for searching for a match to the event; and,
providing for alerting healthcare personnel of the match.

35. A computer readable medium for a system including a memory of patient information to provide decision support to healthcare personnel during treatment of a patient, the medium comprising:
a first code segment for defining an event;
a second code segment for searching for a match to the event; and,
a third code segment for alerting healthcare personnel of the match.

36. A system for scheduling clinical tasks, comprising:
a processor;
a memory being operably connected to the processor, the memory being capable of storing clinical orders;
a search module, the search module being capable of searching the memory for at least partially matching clinical orders;
an electronic system for viewing the matched clinical orders; and,
means for editing selecting and editing the matched clinical orders to create a final order.

37. The system of claim 36, wherein the clinical orders comprise clinical items.

38. The system of claim 36, wherein the clinical orders comprise clinical tests.

39. The system of claim 36, wherein the clinical orders comprise clinical procedures.

40. The system of claim 36, wherein the final order comprises a schedule of activities for a clinician.

41. The system of claim 36, wherein the final order is a exclusive schedule that has orders scheduled, and wherein the scheduling of the orders is not dependent on patient care related events.

42. The system of claim 36, wherein the final order is a interdependent schedule, and wherein the scheduling of the orders is dependent on patient care related events.

43. The system of claim 36, further comprising an alert being activated in response to a scheduled order.

44. The system of claim 36, wherein management of the system is provided in real time.

45. The system of claim 36, wherein communications between system components are performed via a wireless communication link.

46. The system of claim 36, wherein a schedule of the final order is sent to personal digital assistant for viewing by a clinician.

47. The system of claim 36, wherein the final order received by the clinician is integrated in a patient's electronic chart.

48. The system of claim 36, wherein the final order automatically generates a clinician schedule of workflow.

49. The system of claim 36, further comprising means for checking off orders as they are completed.

50. The system of claim 37, further comprising means for checking off items from the schedule.

51. The system of claim 38, further comprising means for checking off tests from the schedule as they are completed.

52. The system of claim 39, further comprising means for checking off procedures from the schedule as they are completed.

53. A system for scheduling clinical tasks, comprising:
a processor;
a memory operably connected to the processor; and,
a record of existing orders being stored in the memory, wherein the processor and the memory cooperate to generate an electronic system for creating a schedule of new orders.

54. The system of claim 53, wherein the user can copy a portion of the existing orders into the new order.
